(19) United States
(12) Patent Application Publication
(10) Pub. No.: US 2021/0238192 A1
(43) Pub. Date: Aug. 5, 2021

(54) VASCULAR ADHESION PROTEIN-1 (VAP-1) MODULATORS AND THERAPEUTIC USES THEREOF

(71) Applicant: Blade Therapeutics, Inc., South San Francisco, CA (US)

(72) Inventors: Brad Owen Buckman, Oakland, CA (US); Prabha Ibrahim, Mountain View, CA (US); Shendong Yuan, San Ramon, CA (US); Kumaraswamy Emayan, Albany, CA (US); Marc Adler, Orinda, CA (US)

(21) Appl. No.: 17/259,673

(22) PCT Filed: Jun. 27, 2019
(86) PCT No.: PCT/US2019/039415

§ 371 Date: Jan. 12, 2021

Related U.S. Application Data
(60) Provisional application No. 62/725,728, filed on Aug. 31, 2018.
(60) Provisional application No. 62/692,451, filed on Jun. 29, 2018.

(51) Int. Cl. C07D 495/04; A61P 19/04; C07C 233/88; C07D 235/06; C07D 235/18; C07C 271/28; C07C 233/25; C07D 317/64; C07C 311/18; C07D 277/24; C07D 233/64; C07D 239/26; C07C 217/46; C07D 271/06; C07C 211/35; C07C 211/27; C07C 237/30; C07D 263/32; C07D 231/12; C07D 257/04; C07D 333/16; C07C 233/36; C07D 209/08; C07D 249/06; C07D 233/22; C07D 249/08; C07C 275/40; C07C 275/28; C07D 401/04

(52) U.S. Cl. CPC C07D 495/04; C07D 401/04; C07C 233/88; C07D 235/06; C07D 235/18; C07C 271/28; C07C 233/25; C07D 317/64; C07C 311/18; C07D 277/24; C07D 233/64; C07D 239/26; C07C 217/46; C07D 271/06; C07C 211/35; C07C 211/27; C07C 237/30; C07D 263/32; C07D 231/12; C07D 257/04; C07D 333/16; C07C 233/36; C07D 209/08; C07D 249/06; C07D 233/22; C07D 249/08; C07C 275/40; C07C 275/28; A61P 19/04

(57) ABSTRACT

Disclosed herein are small molecule Vascular Adhesion Protein-1 (VAP-1) modulator compositions, pharmaceutical compositions, the use and preparation thereof.

### BACKGROUND

### Field of the Invention

The present invention relates to the fields of chemistry and medicine. More particularly, the present invention relates to substituted 3-fluoro-prop-2-en-1-amine compounds as small molecule vascular adhesion protein-1 (VAP-1) modulators, compositions, their preparation, and their use as therapeutic agents.

### Description of the Related Art

Semicarbazide-sensitive amino oxidase/vascular adhesion protein-1 (SSAO)/VAP-1) is a member of the semicarbazide-sensitive amino oxidase family. SSAO/VAP-1 has been alternatively referred to as VAP-1 or SSAO or AOC3 (Amine oxidase, copper containing 3). SSAO/VAP-1 is an enzyme that exists both as a membrane-bound and a soluble isoform; it is predominantly expressed from endothelial cell surface, vascular smooth muscle and adipose cells. SSAO/VAP-1 participates in many cellular processes including glucose disposition, inflammation responses, and leukocyte recruitment. High activity levels of this enzyme are associated with diabetes, atherosclerosis, strokes, chronic kidney disease, and fatty liver disease, among other disorders. SSAO/VAP-1 has been implicated in the pathogenesis of liver diseases such as fatty liver disease. Fatty liver disease (FLD) encompasses a spectrum of disease states where fat accumulates in the liver in excessive amounts and is accompanied by inflammation. FLD can lead to non-alcoholic fatty liver disease (NAFLD), which is characterized by insulin resistance. Individuals experiencing chronic liver inflammation often develop liver fibrosis, with eventual risks of cirrhosis, hepatocellular carcinoma, and liver failure

Currently there is a need to provide alternative treatment therapies for liver diseases such as NAFLD and/or NASH. A SSAO/VAP-1 inhibitor will reduce liver inflammation and fibrosis and thereby provide a treatment for liver diseases, in particular, a treatment for NAFLD and/or NASH. Based on certain projections, NASH is predicted to become the leading cause of liver transplantation by 2020. Unfortunately, there are no therapies available to prevent or treat liver fibrosis.

Fibrotic disease accounts for an estimated 45% of deaths in the developed world but the development of therapies for such diseases is still in its infancy. The current treatments for fibrotic diseases, such as for diabetic nephropathy, idiopathic lung fibrosis, renal fibrosis, systemic sclerosis, idiopathic pulmonary fibrosis, progressive liver fibrosis, non-alcoholic steatohepatitis, primary sclerosing cholangitis, corneal fibrosis, and liver cirrhosis, are few in number and only alleviate some of the symptoms of fibrosis while failing to treat the underlying cause.

### SUMMARY

A compound having the structure of the formula (I):

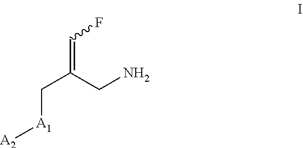

or a pharmaceutically acceptable salt thereof, wherein:

A₁ is selected from the group consisting of —(CH₂)ₙ—, —S—, —S(═O)—, —SO₂—, —O—, —C(═S)—, —C(═O)—, —NR⁵—, —C(O)NR⁵—, —S(CH₂)ₙ—, —O(CH₂)ₙ—, —NR⁵(CH₂)ₙ—, —OC(O)NR⁵—, —NHC(O)NH— —NHC(S)NH—, —NHC(S)O—, —NHC(S)—, —NR⁵SO₂—, and a bond;

when A₁ is —(CH₂)₂—, —S(═O)—, —SO₂—, —C(═S)—, —C(═O)—, —NR⁵—, —C(O)NR⁵—, —S(CH₂)ₙ—, —O(CH₂)ₙ—, —NR⁵(CH₂)ₙ—, —OC(O)NR⁵—, —NHC(O)NH— —NHC(S)NH—, —NHC(S)O—, —NHC(S)—, or —NR⁵SO₂—, A₂ is selected from the group consisting of 3-10 membered heterocyclyl optionally substituted with one or more R⁴, C₆₋₁₀ aryl optionally substituted with one or more R⁴, 5-10 membered heteroaryl optionally substituted with one or more R⁴, C₃₋₁₀ carbocyclyl optionally substituted with one or more R⁴, and C₆₋₁₀ aryl(C₁-C₆)alkyl optionally substituted with one or more R⁴

when A₁ is a bond, A₂ is selected from the group consisting of —SO₂NR⁵R⁶, —(CH₂)ₙSO₂NR⁵R⁶, —(CH₂)ₙNR⁵SO₂R⁶, —NRSO₂R⁶, C₆₋₁₀ aryl substituted with one or more R³, 3-10 membered heterocyclyl optionally substituted with one or more R⁴, C₃₋₁₀ carbocyclyl optionally substituted with one or more R^{3a}, and —NR¹R² wherein R¹ and R² together with the nitrogen to which they are attached form a 3-10 membered heterocyclyl optionally substituted with one or more R⁴ or a 5-10 membered heteroaryl optionally substituted with one or more R⁴;

when A₁ is —O—, A₂ is selected from the group consisting of 3-10 membered heterocyclyl optionally substituted with one or more R⁴, C₆₋₁₀ aryl substituted with one or more R^{3a}, 5-10 membered heteroaryl optionally substituted with one or more R⁷, C₃₋₁₀ carbocyclyl optionally substituted with one or more R⁴, and C₆₋₁₀ aryl(C₁-C₆)alkyl optionally substituted with one or more R⁴;

when A₁ is —(CH₂)ₙ—, A₂ is selected from the group consisting of 3-10 membered heterocyclyl optionally substituted with one or more R⁴, C₆₋₁₀ aryl substituted with one or more R^{3a}, 5-10 membered heteroaryl optionally substituted with one or more R⁷, C₃₋₁₀ carbocyclyl optionally substituted with one or more R⁴, and C₆₋₁₀ aryl(C₂-C₆)alkyl optionally substituted with one or more R⁴; and

when A₁ is —S—, A₂ is selected from the group consisting of 3-10 membered heterocyclyl optionally substituted with one or more R⁴, C₆₋₁₀ aryl substituted with one or more R^{3a}, 5-10 membered heteroaryl optionally substituted with one or more R⁴, C₃₋₁₀ carbocyclyl optionally substituted with one or more R⁴, and C₆₋₁₀ aryl(C₁-C₆)alkyl optionally substituted with one or more R⁴;

each R³ is independently selected from 5-10 membered heterocyclyl (optionally substituted with —OH, halo, C₁-C₆ alkyl, C₁-C₆ alkoxy, C₁-C₆ haloalkyl, and C₁-C₆ haloalkoxy), 5-10 membered heteroaryl (optionally substituted with —OH, halo, C₁-C₆ alkyl, C₁-C₆ alkoxy, C₁-C₆ haloalkyl, and C₁-C₆ haloalkoxy), —NR⁵COOR⁶, —NR⁵COR⁶, —NR⁵CONR⁵R⁶, —NR⁵CSNR⁵R⁶, —O(CO)NR⁵R⁶, and C₃₋₇ carbocyclyl (optionally substituted with —OH, halo, C₁-C₆ alkyl, C₁-C₆ alkoxy, C₁-C₆ haloalkyl, and C₁-C₆ haloalkoxy);

each R^{3a} is independently selected from 5-10 membered heterocyclyl (optionally substituted with —OH, halo, C₁-C₆ alkyl, C₁-C₆ alkoxy, C₁-C₆ haloalkyl, and C₁-C₆ haloalkoxy), aryl (optionally substituted with —OH, halo, C₁-C₆ alkyl, C₁-C₆ alkoxy, C₁-C₆ haloalkyl, and C₁-C₆ haloalkoxy), 5-10 membered heteroaryl (optionally substituted with —OH, halo, C₁-C₆ alkyl, C₁-C₆ alkoxy, C₁-C₆ haloalkyl, and C₁-C₆ haloalkoxy), —NR⁵COOR⁶, —NR⁵COR⁶, —NR⁵CONR⁵R⁶, —NR⁵CSNR⁵R⁶, —O(CO)NR⁵R⁶, and C₃₋₇ carbocyclyl (optionally substituted with —OH, halo, C₁-C₆ alkyl, C₁-C₆ alkoxy, C₁-C₆ haloalkyl, and C₁-C₆ haloalkoxy);

each R⁴ is independently selected from the group consisting of C₁-C₆ alkyl, C₁-C₆ alkenyl, C₁-C₆ alkynyl, C₁-C₆ heteroalkyl, C₃-C₇ carbocyclyl (optionally substituted with —OH, halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with —OH, halo, C₁-C₆ alkyl, C₁-C₆ alkoxy, C₁-C₆ haloalkyl, and C₁-C₆ haloalkoxy), 5-10 membered heterocyclyl (optionally substituted with —OH, halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with —OH, halo, C₁-C₆ alkyl, C₁-C₆ alkoxy, C₁-C₆ haloalkyl, and C₁-C₆ haloalkoxy), aryl (optionally substituted with —OH, halo, C₁-C₆ alkyl, C₁-C₆ alkoxy, C₁-C₆ haloalkyl, and C₁-C₆ haloalkoxy), C₆₋₁₀ aryl(C₁-C₆)alkyl (optionally substituted with —OH, halo, C₁-C₆ alkyl, C₁-C₆ alkoxy, C₁-C₆ haloalkyl, and C₁-C₆ haloalkoxy), 5-10 membered heteroaryl (optionally substituted with —OH, halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with —OH, halo, C₁-C₆ alkyl, C₁-C₆ alkoxy, C₁-C₆ haloalkyl, and C₁-C₆ haloalkoxy), halo, cyano, C₁-C₆ alkoxy, C₁-C₆ alkoxy(C₁-C₆)alkyl), —NR⁵COOR⁶, —NR⁵COR⁶, —NR⁵CONR⁵R⁶, —NR⁵CSNR⁵R⁶, —O(CO)NR⁵R⁶, —NR⁵R⁶, —SO₂NR⁵R⁶, —C(O)NR⁵R⁶, —NSO₂R⁶, —OCF₃, CF₃, —OH, and —SR⁵;

each R⁵ and R⁶ are independently selected from the group consisting of —H, optionally substituted C₁₋₄ alkyl, —CO-(optionally substituted C₁₋₄ alkyl), —CO-(optionally substituted C₆₋₁₀ aryl), optionally substituted C₁₋₈ alkoxyalkyl, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;

R⁷ is independently selected from the group consisting of C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), C₆₋₁₀ aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, C₁-C₆ alkoxy, C₁-C₆ alkoxy(C₁-C₆)alkyl), —NR⁵COOR⁶, —NR⁵CONR⁵R⁶, —O(CO)NR⁵R⁶, —NR⁵R⁶, —SO₂NR⁵R⁶, —C(O)NR⁵R⁶, —NSO₂R⁶, —OCF₃, CF₃, —OH, and —SR⁵; and n is selected to be an integer from 1 to 2.

Other embodiments disclosed herein include a pharmaceutical composition comprising a therapeutically effective amount of a compound disclosed herein and a pharmaceutically acceptable excipient.

In some embodiments, the substituted 3-fluoro-prop-2-en-1-amine compounds disclosed herein are broadly effective in treating a host of conditions arising from fibrosis or inflammation, and specifically including those associated with myofibroblast differentiation. Accordingly, compounds disclosed herein are active therapeutics for a diverse set of diseases or disorders that include or that produces a symptom which include, but are not limited to: progressive liver fibrosis, renal fibrosis, idiopathic lung fibrosis, diabetic nephropathy, systemic sclerosis, idiopathic pulmonary fibrosis, non-alcoholic steatohepatitis, primary sclerosing cholangitis, corneal fibrosis, liver cirrhosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis. In other embodiments, the compounds disclosed herein can be used can be used in metabolic and reaction kinetic studies, detection and imaging techniques and radioactive treatments.

In some embodiments, the compounds disclosed herein are used to treat diseases or conditions or that produces a symptom in a subject which include, but not limited to: progressive liver fibrosis, renal fibrosis, idiopathic lung fibrosis, diabetic nephropathy, systemic sclerosis, idiopathic pulmonary fibrosis, non-alcoholic steatohepatitis, primary sclerosing cholangitis, corneal fibrosis, liver cirrhosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis.

In certain embodiments methods are provided for alleviating or ameliorating a condition or disorder, affected at least in part by the enzymatic activity of vascular adhesion protein-1 (VAP-1), or mediated at least in part by the enzymatic activity of vascular adhesion protein-1 (VAP-1) wherein the condition includes or produces a symptom which includes: progressive liver fibrosis, renal fibrosis, idiopathic lung fibrosis, diabetic nephropathy, systemic sclerosis, idiopathic pulmonary fibrosis, non-alcoholic steatohepatitis, primary sclerosing cholangitis, corneal fibrosis, liver cirrhosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis.

In some embodiments, the methods, compounds, and/or compositions of the present invention are used for prophylactic therapy.

### DETAILED DESCRIPTION

In some embodiments, compounds that are non-macrocyclic (α-keto amides are provided that act as VAP-1 modulators. Various embodiments of these compounds include compounds having the structures of Formula I as described above or pharmaceutically acceptable salts thereof. The structure of Formula I encompasses all stereoisomers and racemic mixtures, including the following structures and mixtures thereof:

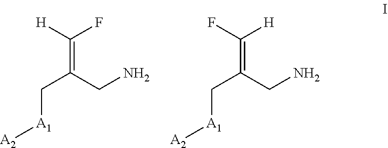

In some embodiments of compounds of Formula (I):

A₁ is selected from the group consisting of —(CH₂)ₙ—, —S—, —S(═O)—, —SO₂—, —O—, —C(═S)—, —C(═O)—, —NR⁵—, —C(O)NR⁵—, —S(CH₂)ₙ—, —O(CH₂)ₙ—, —NR⁵(CH₂)ₙ—, —OC(O)NR⁵—, —NHC(O)NH— —NHC(S)NH—, —NHC(S)O—, —NHC(S)—, —NR⁵SO₂—, and a bond; when A₁ is —(CH₂)₂—, —S(═O)—, —SO₂—, —C(═S)—, —C(═O)—, —NR⁵—, —C(O)NR⁵—, —S(CH₂)ₙ—, —O(CH₂)ₙ—, —NR⁵(CH₂)ₙ—, —OC(O)NR⁵—, —NHC(O)NH—, —NHC(S)NH—, —NHC(S)O—, —NHC(S)—, or —NR⁵SO₂—, A₂ is selected from the group consisting of 3-10 membered heterocyclyl optionally substituted with one or more R⁴, C₆₋₁₀ aryl optionally substituted with one or more R⁴, 5-10 membered heteroaryl optionally substituted with one or more R⁴, C₃₋₁₀ carbocyclyl optionally substituted with one or more R⁴, and C₆₋₁₀ aryl(C₁-C₆)alkyl optionally substituted with one or more R⁴;

when A₁ is a bond, A₂ is selected from the group consisting of —SO₂NR⁵R⁶, —(CH₂)ₙSO₂NR⁵R⁶, —(CH₂)ₙNR⁵SO₂R⁶, —NR⁵SO₂R⁶, C₆₋₁₀ aryl substituted with one or more R³, 3-10 membered heterocyclyl optionally substituted with one or more R⁴, C₃₋₁₀ carbocyclyl optionally substituted with one or more R³, and —NR¹R² wherein R¹ and R² together with the nitrogen to which they are attached form a 3-10 membered heterocyclyl optionally substituted with one or more R⁴ or a 5-10 membered heteroaryl optionally substituted with one or more R⁴;

when A₁ is —O—, A₂ is selected from the group consisting of 3-10 membered heterocyclyl optionally substituted with one or more R⁴, C₆₋₁₀ aryl substituted with one or more R^{3a}, 5-10 membered heteroaryl optionally substituted with one or more R⁷, C₃₋₁₀ carbocyclyl optionally substituted with one or more R⁴, and C₆₋₁₀ aryl(C₁-C₆)alkyl optionally substituted with one or more R⁴;

when A₁ is —(CH₂)ₙ—, A₂ is selected from the group consisting of 3-10 membered heterocyclyl optionally substituted with one or more R⁴, C₆₋₁₀ aryl substituted with one or more R^{3a}, 5-10 membered heteroaryl optionally substituted with one or more R⁷, C₃₋₁₀ carbocyclyl optionally substituted with one or more R⁴, and C₆₋₁₀ aryl(C₂-C₆)alkyl optionally substituted with one or more R⁴; and

when A₁ is —S—, A₂ is selected from the group consisting of 3-10 membered heterocyclyl optionally substituted with one or more R⁴, C₆₋₁₀ aryl substituted with one or more R^{3a}, 5-10 membered heteroaryl optionally substituted with one or more R⁴, C₃₋₁₀ carbocyclyl optionally substituted with one or more R⁴, and C₆₋₁₀ aryl(C₁-C₆)alkyl optionally substituted with one or more R⁴;

each R³ is independently selected from 5-10 membered heterocyclyl (optionally substituted with —OH, halo, C₁-C₆ alkyl, C₁-C₆ alkoxy, C₁-C₆ haloalkyl, and C₁-C₆ haloalkoxy), 5-10 membered heteroaryl (optionally substituted withOH, halo, C₁-C₆ alkyl, C₁-C₆ alkoxy, C₁-C₆ haloalkyl, and C₁-C₆ haloalkoxy), —NR⁵COOR⁶, —NR⁵COR⁶, —NR⁵CONR⁵R⁶, —NR⁵CSNR⁵R⁶, —O(CO)NR⁵R⁶, and C₃₋₇ carbocyclyl (optionally substituted with —OH, halo, C₁-C₆ alkyl, C₁-C₆ alkoxy, C₁-C₆ haloalkyl, and C₁-C₆ haloalkoxy); each R^{3a} is independently selected from 5-10 membered heterocyclyl (optionally substituted with —OH, halo, C₁-C₆ alkyl, C₁-C₆ alkoxy, C₁-C₆ haloalkyl, and C₁-C₆ haloalkoxy), aryl (optionally substituted with —OH, halo, C₁-C₆ alkyl, C₁-C₆ alkoxy, C₁-C₆ haloalkyl, and C₁-C₆ haloalkoxy), 5-10 membered heteroaryl (optionally substituted with —OH, halo, C₁-C₆ alkyl, C₁-C₆ alkoxy, C₁-C₆ haloalkyl, and C₁-C₆ haloalkoxy), —NR⁵COOR⁶, —NR⁵COR⁶, —NR⁵CONR⁵R⁶, —NR⁵CSNR⁵R⁶, —O(CO)NR⁵R⁶, and C₃₋₇ carbocyclyl (optionally substituted with —OH, halo, C₁-C₆ alkyl, C₁-C₆ alkoxy, C₁-C₆ haloalkyl, and C₁-C₆ haloalkoxy);

each R⁴ is independently selected from the group consisting of C₁-C₆ alkyl, C₁-C₆ alkenyl, C₁-C₆ alkynyl, C₁-C₆ heteroalkyl, C₃-C₇ carbocyclyl (optionally substituted with —OH, halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with —OH, halo, C₁-C₆ alkyl, C₁-C₆ alkoxy, C₁-C₆ haloalkyl, and C₁-C₆ haloalkoxy), 5-10 membered heterocyclyl (optionally substituted with —OH, halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with —OH, halo, C₁-C₆ alkyl, C₁-C₆ alkoxy, C₁-C₆ haloalkyl, and C₁-C₆ haloalkoxy), aryl (optionally substituted with —OH, halo, C₁-C₆ alkyl, C₁-C₆ alkoxy, C₁-C₆ haloalkyl, and C₁-C₆ haloalkoxy), C₆₋₁₀ aryl(C₁-C₆)alkyl (optionally substituted with —OH, halo, C₁-C₆ alkyl, C₁-C₆ alkoxy, C₁-C₆ haloalkyl, and C₁-C₆ haloalkoxy), 5-10 membered heteroaryl (optionally substituted with —OH, halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with —OH, halo, C₁-C₆ alkyl, C₁-C₆ alkoxy, C₁-C₆ haloalkyl, and C₁-C₆ haloalkoxy), halo, cyano, C₁-C₆ alkoxy, C₁-C₆ alkoxy(C₁-C₆)alkyl), —NR⁵COOR⁶, —NR⁵COR⁶, —NR⁵CONR⁵R⁶, —NR⁵CSNR⁵R⁶, —O(CO)NR⁵R⁶, —NR⁵R⁶, —SO₂NR⁵R⁶, —C(O)NR⁵R⁶, —NSO₂R⁶, —OCF₃, CF₃, —OH, and —SR⁵;

each R⁵ and R⁶ are independently selected from the group consisting of —H, optionally substituted C₁₋₄ alkyl, —CO-(optionally substituted C₁₋₄ alkyl), —CO-(optionally substituted C₆₋₁₀ aryl), optionally substituted C₁₋₆ alkoxyalkyl, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;

R⁷ is independently selected from the group consisting of C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), C₆₋₁₀ aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, C₁-C₆ alkoxy, C₁-C₆ alkoxy(C₁-C₆)alkyl), —NR⁵COOR⁶, —NR⁵CONR⁵R⁶, —O(CO)NR⁵R⁶, —NR⁵R⁶, —SO₂NR⁵R⁶, —C(O)NR⁵R⁶, —NSO₂R⁶, —OCF₃, CF₃, —OH, and —SR⁵; and n is selected to be an integer from 1 to 2.

Some embodiments of compounds of Formula (I) include compounds having the structure of Formula (I-a):

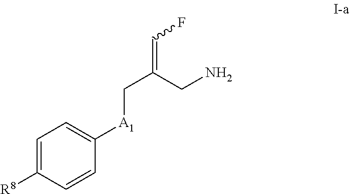

or a pharmaceutically acceptable salt thereof, wherein:
A₁ is selected from the group consisting of —CH₂—, O, NH, S, —NHCONH—, —C(O)NR⁵—, —NR⁵SO₂—, and —O(CO)NH—; and

R⁸ is selected from the group consisting of —NR⁵COOR⁶, —NR⁵COR⁶, —NR⁵CONR⁵R⁶, —O(CO)NR⁵R⁶, C₆₋₁₀ aryl substituted with one or more R³, and 5-10 membered heteroaryl (optionally substituted with one or more —OH, halo, C₁-C₆ alkyl, or C₁-C₆ alkoxy).

In some embodiments of compounds of Formula (I-a) or their pharmaceutically acceptable salts; A₁ is selected from the group consisting of —O—, —NH—, and —S—.

In some embodiments of compounds of Formula (I-a) or their pharmaceutically acceptable salts; R⁸ is selected from the group consisting of —NHCONHⁱPr, —NHCONEt₂, —N(Me)CONHEt, —NHCOOEt, —NHCOEt, and —NHCONHEt.

In some embodiments of compounds of Formula (I-a) or their pharmaceutically acceptable salts; R⁸ is selected from the group consisting of

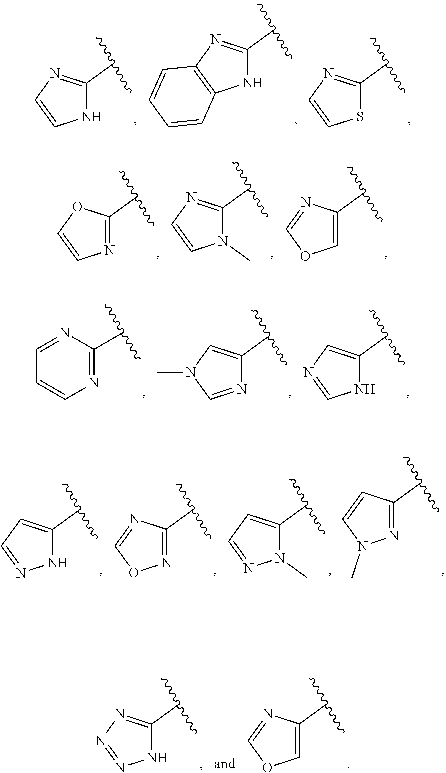

Some embodiments of compounds of Formula (I) include compounds having the structure of Formula (I-b):

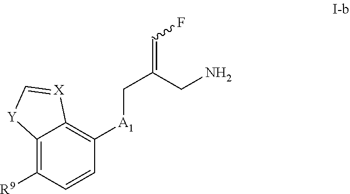

or a pharmaceutically acceptable salt thereof, wherein:

A₁ is selected from the group consisting of —CH₂—, —O—, NH, —S—, —SO₂, —NHCONH—, and —O(CO)NH—; X is selected from the group consisting of ═N— and —CH—; Y is selected from the group consisting of —NR—, —O—, and —S—; and R⁹ is selected from the group consisting of —C(O)NR⁵R⁶, —NR⁵SCOOR⁶, —NR⁵CONR⁵SR⁶, —O(CO)NR⁵R⁶, and —SO₂NR⁵R⁶.

In some embodiments of compounds of Formula (I-b) or their pharmaceutically acceptable salts; X is ═N— and Y is —NH—.

In some embodiments of compounds of Formula (I-b) or their pharmaceutically acceptable salts; X is ═N— and Y is —S—.

Some embodiments of compounds of Formula (I) include compounds having the structure of Formula (I-c):

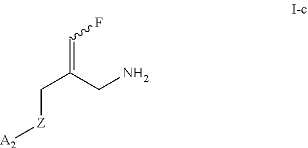

or a pharmaceutically acceptable salt thereof, wherein:

Z is selected from the group consisting of O, —OCH₂—, —NR⁵S, —NR⁵(CH₂)ₙ—, —S—, —S(═O)—, —SO₂—, —C(═S)—, —C(═O)—, —C(O)NR⁵—, —S(CH₂)ₙ—, —OC(O)NR⁵—, —NHC(O)NH— —NHC(S)NH—, —NHC(S)O—, —NHC(S)—, and —NR⁵SO₂—; and A₂ is selected from the group consisting of 3-10 membered heterocyclyl optionally substituted with one or more R⁴, C₆₋₁₀ aryl substituted with one or more R³, 5-10 membered heteroaryl optionally substituted with one or more R⁷, C₃₋₁₀ carbocyclyl optionally substituted with one or more R⁴, and C₆₋₁₀ aryl(C₁-C₆)alkyl optionally substituted with one or more R⁴.

In some embodiments of compounds of Formula (I-c), Z is selected from the group consisting of —O—, —NH, —NCOCH₃, and —OC(O)NH—.

In some embodiments of compounds of Formula (I-c), A₂ is

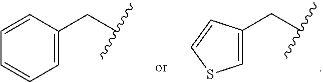 [SMILES: *Cc1ccccc1 ; *Cc1ccsc1]

Some embodiments of compounds of Formula (I) include compounds having the structure of formula (I-d):

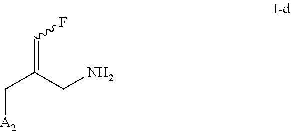

or a pharmaceutically acceptable salt thereof, wherein:

A₂ is selected from the group consisting of —SO₂NR⁵R⁶, —(CH₂)ₙSO₂NR⁵R⁶, —(CH₂)ₙNR⁵SO₂R⁶, and —NR⁵SO₂R⁶.

In some embodiments of compounds of Formula (I-d) or their pharmaceutically acceptable salts; A₂ is selected from the group consisting of —NHSO₂Me, and —NHSO₂Ph.

Some embodiments of compounds of Formula (I) include compounds having the structure of Formula (I-e):

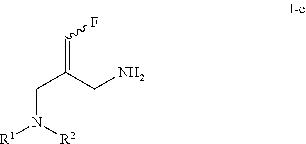

or a pharmaceutically acceptable salt thereof, wherein:

NR¹R² wherein R¹ and R² together with the nitrogen to which they are attached form a 3-10 membered heterocyclyl optionally substituted with one or more R⁴ or a 5-10 membered heteroaryl optionally substituted with one or more R⁴.

In some embodiments of compounds of Formula (I-e) or their pharmaceutically acceptable salts; wherein R¹ and R² together with the nitrogen to which they are attached form a ring selected from the group consisting of

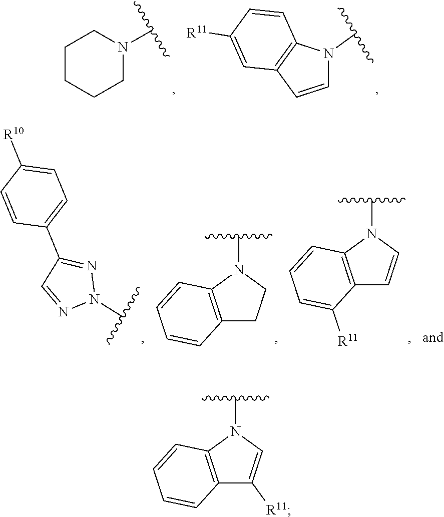

R¹¹ is selected from the group consisting of H, —OH, —C(O)NR⁵R⁶, —NR⁵SCOOR⁶, —NR⁵CONR⁵R⁶, and —O(CO)NR⁵R⁶, and —SO₂NR⁵R⁶, —NR⁵R⁶, —NSO₂R⁶, —NR⁵COR⁶, —OCF₃, —CF₃, and —SR⁵ and R¹⁰ is selected from the group consisting of H, —OH, halo, C₁-C₆ alkyl, C₁-C₆ alkoxy, C₁-C₆ haloalkyl, and C₁-C₆ haloalkoxy.

In some embodiments of compounds of Formula (I-e), R¹¹ is selected from the group consisting of H, —C(O)NR⁵R⁶, —NR⁵COOR⁶, —NR⁵CONR⁵R⁶, and —O(CO)NR⁵R⁶, and —SO₂NR⁵R⁶.

Some embodiments of compounds of Formula (I) include compounds having the structure of Formula (I-f):

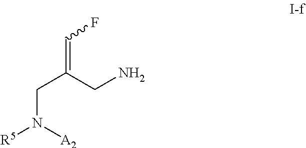

or a pharmaceutically acceptable salt thereof, wherein:

R⁵ is selected from —H, optionally substituted C₁₋₄ alkyl, —CO-(optionally substituted C₁₋₄ alkyl), —CO-(optionally substituted C₆₋₁₀ aryl), optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl; and

R⁴ is selected from halo, cyano, hydroxy, C₁-C₆ alkoxy, C₁-C₆ alkoxy(C₁-C₆)alkyl), —NR⁵COOR⁶, —NR⁵COR⁶, —NR⁵CONR⁵R⁶, —O(CO)NR⁵R⁶, —NR⁵R⁶, —SO₂NR⁵R⁶, —C(O)NR⁵R⁶, —NSO₂R⁶, —OCF₃, CF₃, —OH, and —SR⁵.

In some embodiments of compounds of Formula (I-f) or their pharmaceutically acceptable salts; R⁵ is selected from the group consisting of H, —COMe, —COPh, —CH₂Ph, phenyl, and cyclohexyl.

In some embodiments of compounds of Formula (I-f), R⁴ is —CONH^{t}Bu.

In some embodiments of compounds of Formula (I), A₁ is selected from the group consisting of —S—, —S(═O)—, —SO₂—, —O—, —C(═S)—, —C(═O)—, —NR⁵—, —C(O)NR⁵—, —S(CH₂)ₙ—, —O(CH₂)ₙ—, —NR⁵(CH₂)ₙ—, —OC(O)NR⁵—, —NHC(O)NH— —NHC(S)NH—, —NHC(S)O—, —NHC(S)—, and —NR⁵SO₂—.

Some embodiments include a compound selected from the group consisting of:

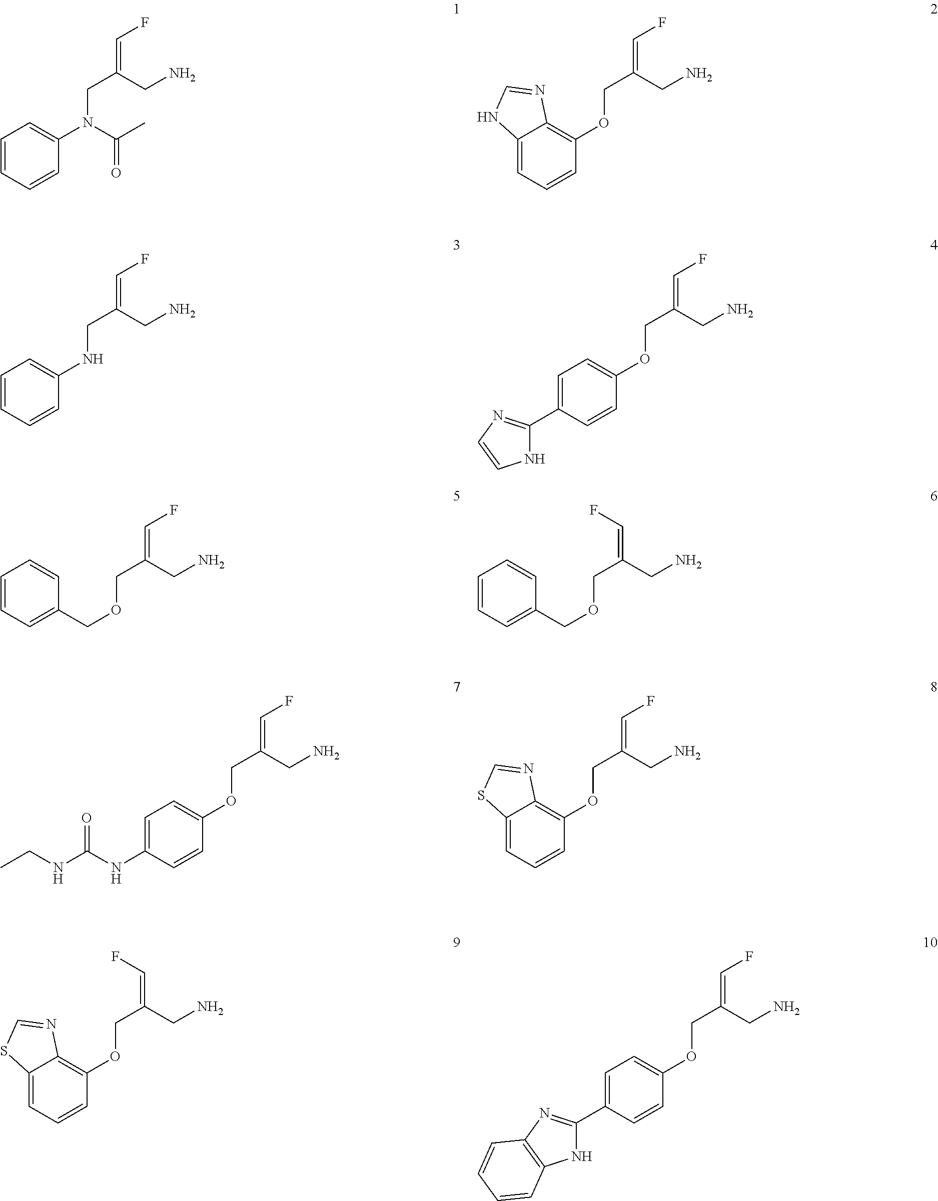

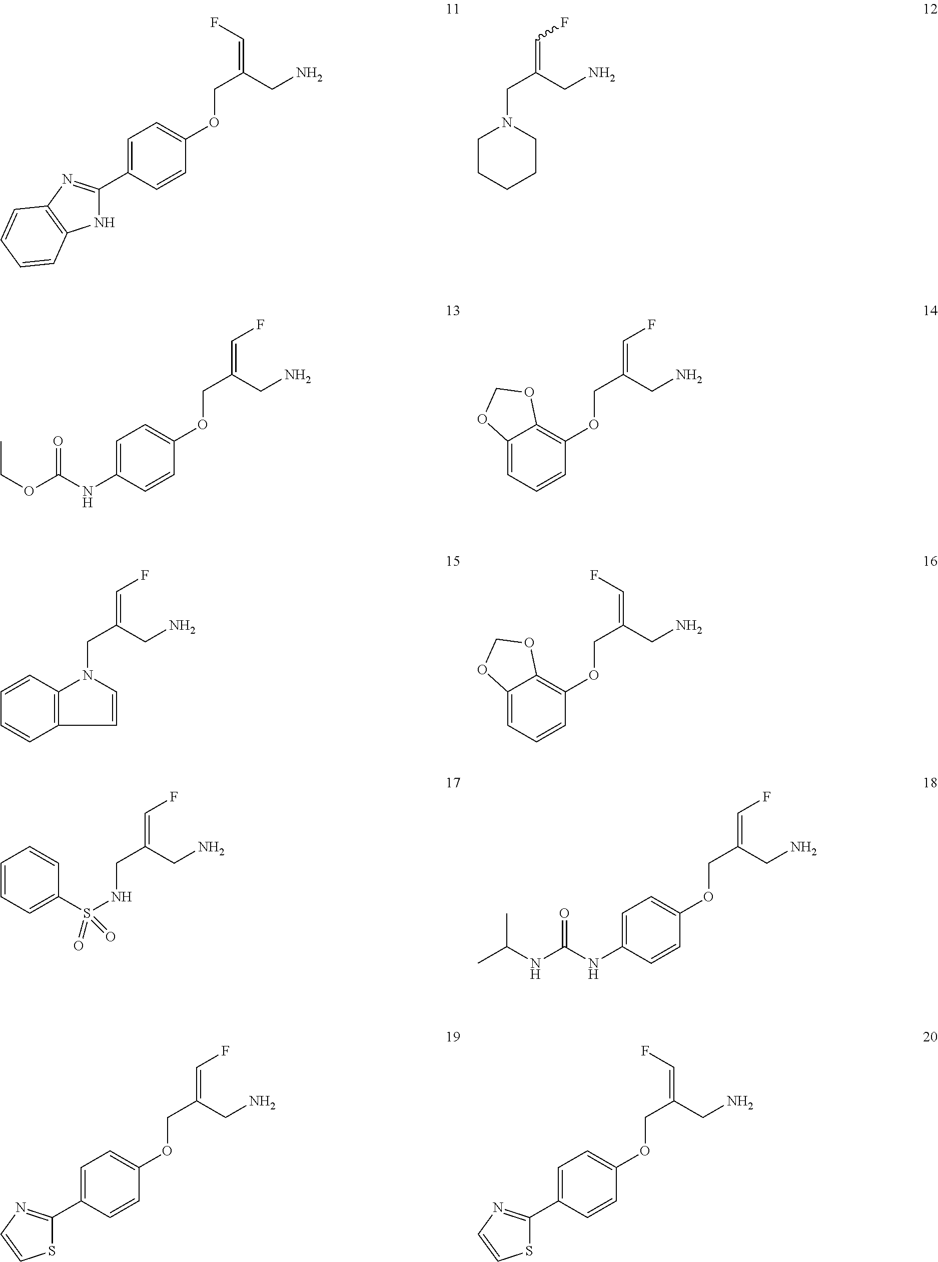

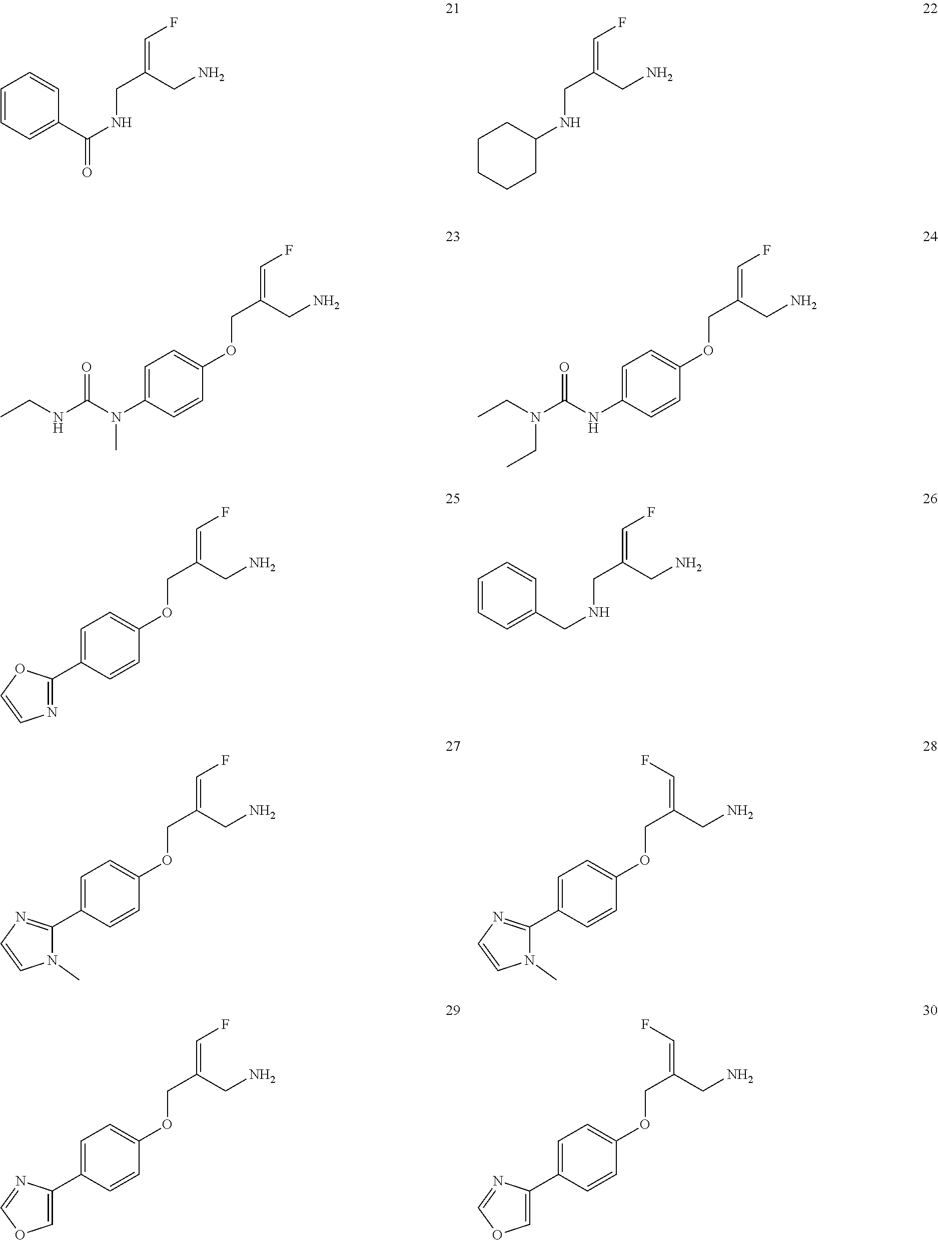

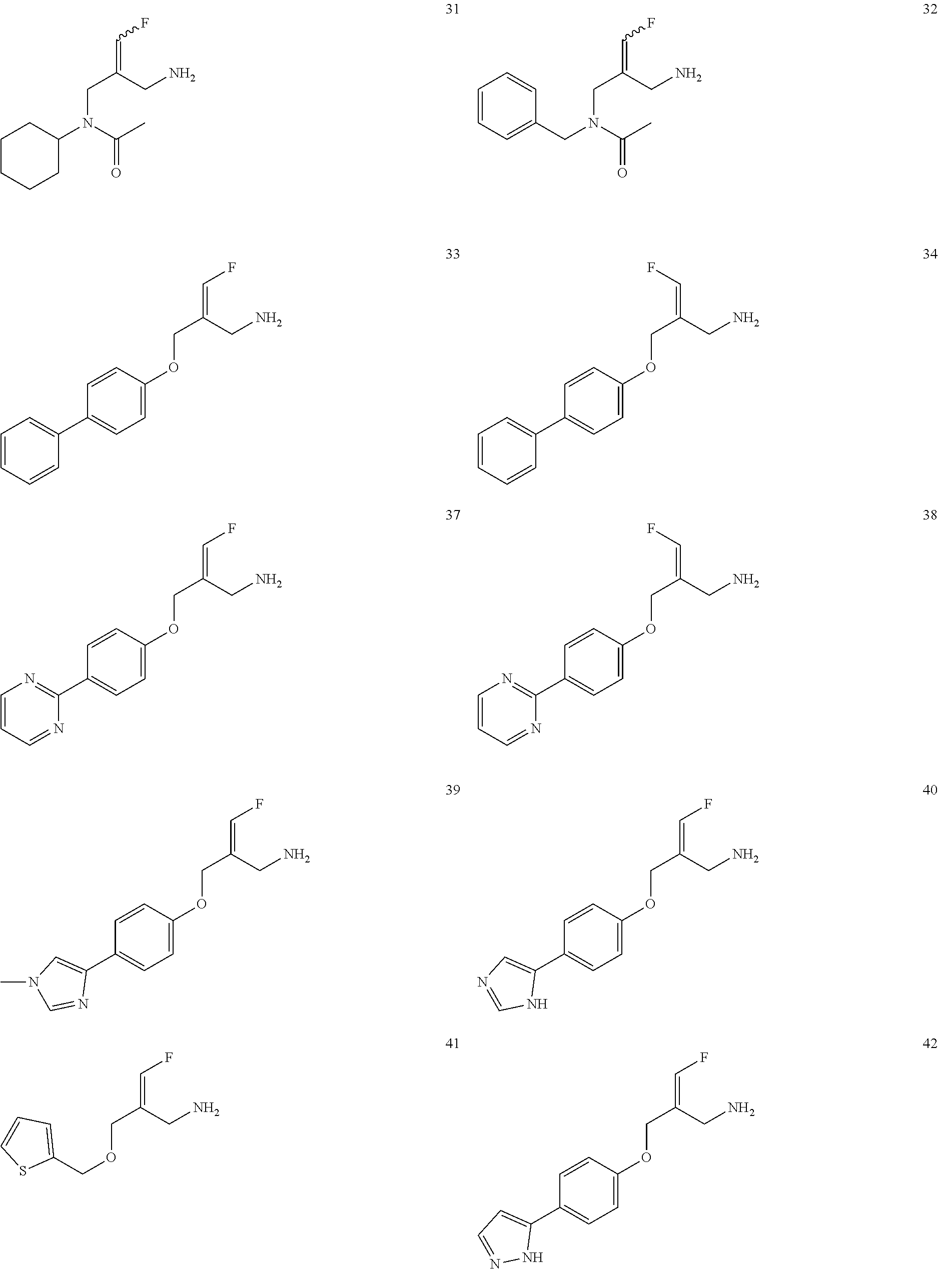

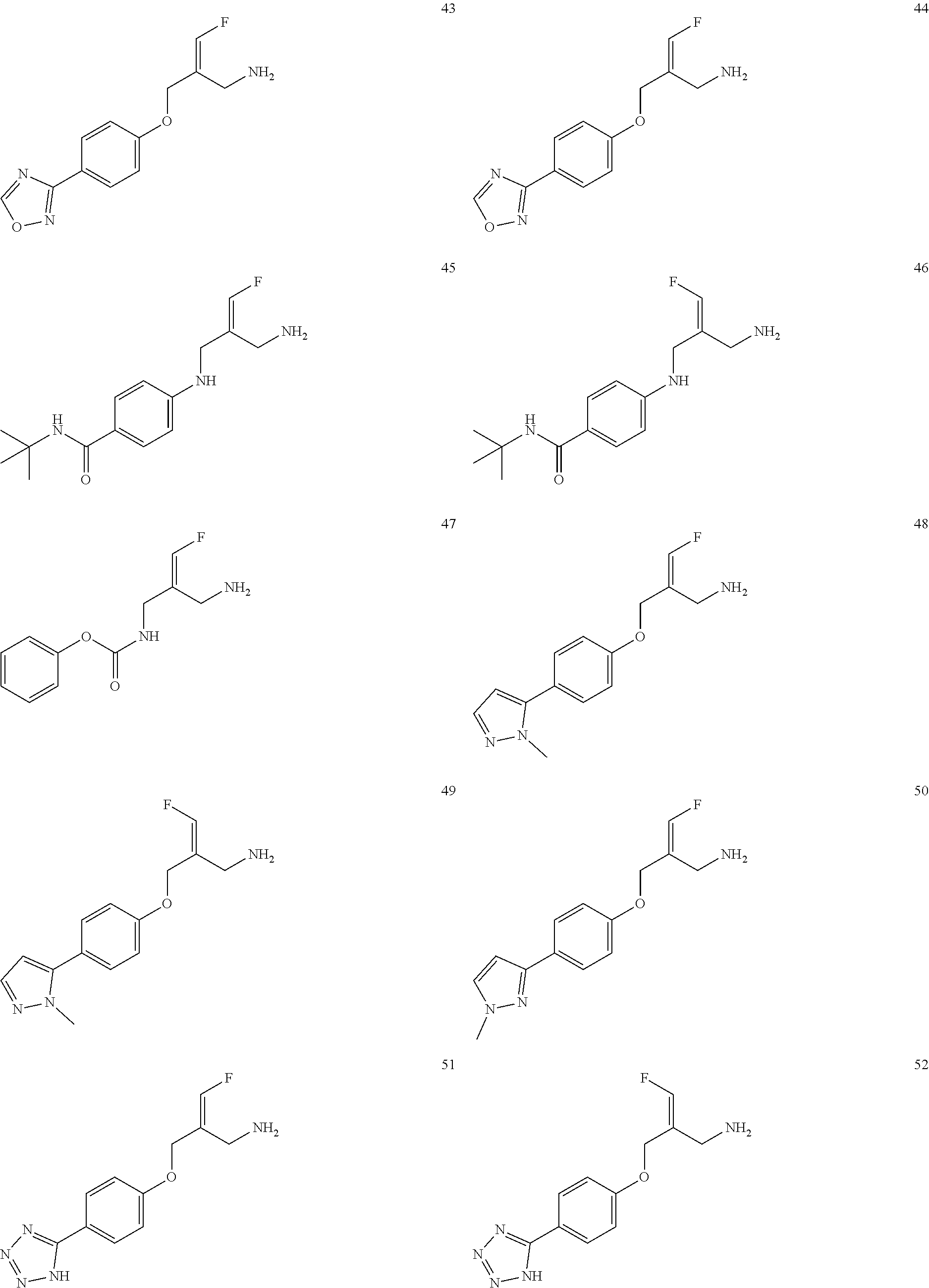

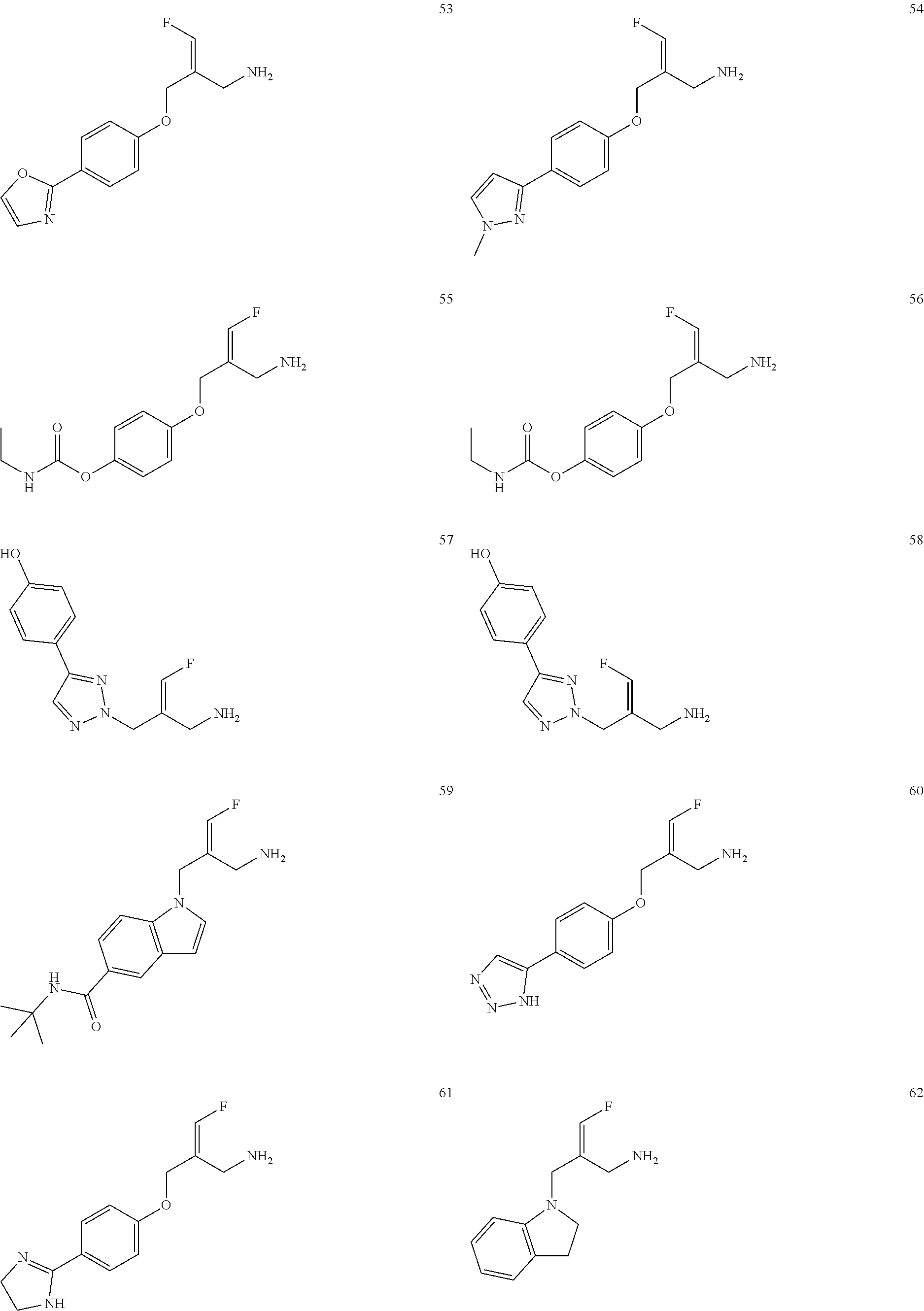

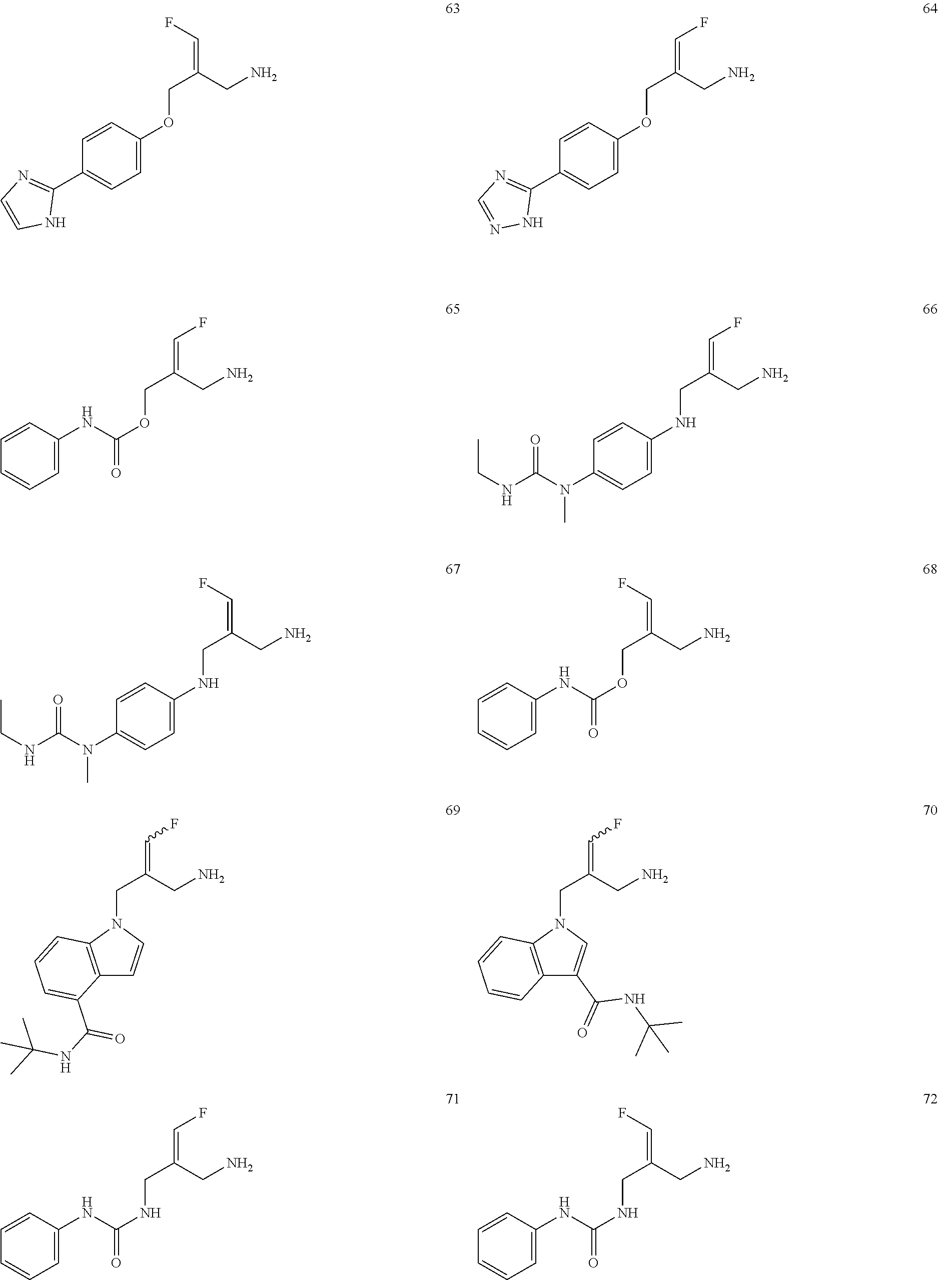

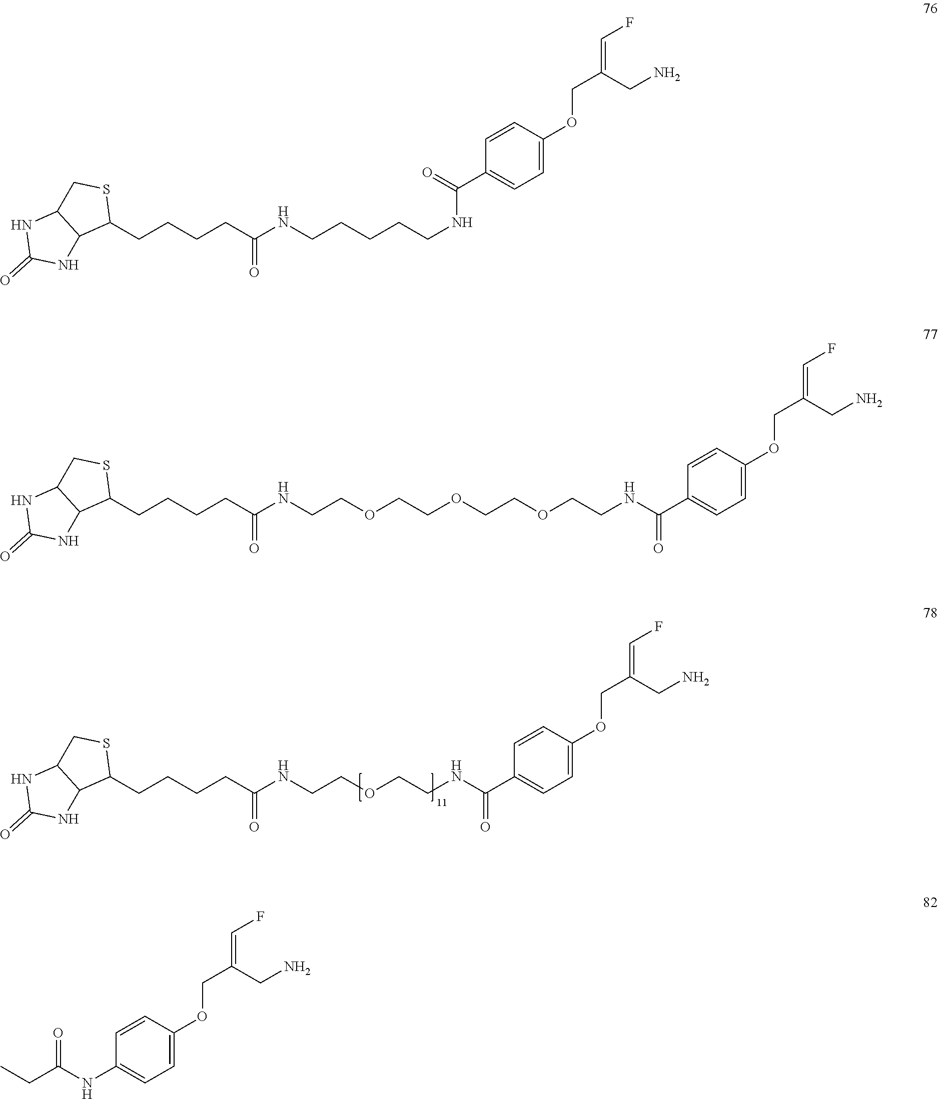

and pharmaceutically acceptable salts thereof.

Some embodiments include a compound having the structure selected from:

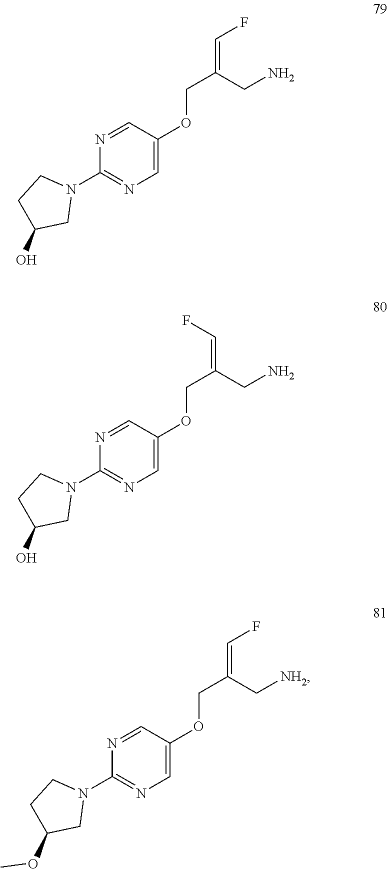 [SMILES: NC/C(=C\F)COc1cnc(N2CC[C@H](O)C2)nc1 ; NC/C(=C/F)COc1cnc(N2CC[C@H](O)C2)nc1 ; CO[C@H]1CCN(c2ncc(OC/C(=C/F)CN)cn2)C1]

pharmaceutically acceptable salts thereof.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

### Isotopically-Labeled Compounds

Isotopes may be present in the compounds described. Each chemical element as represented in a compound structure may include any isotope of said element. The isotopes may be isotopes of carbon, chlorine, fluorine, hydrogen, iodine, nitrogen, oxygen, phosphorous, sulfur, and technetium, including ¹¹C, ¹³C, ¹⁴C, ³⁶Cl, ¹⁸F, ²H, ³H, ¹²³I, ¹²⁵I, ¹³N, ¹⁵N, ¹⁵O, ¹⁷O, ¹⁸O, ³¹P, ³²P, ³⁵S, and ^{99m}Tc. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Isotopically-labeled compounds of the present embodiments are useful in drug and substrate tissue distribution and target occupancy assays. For example, isotopically labeled compounds are particularly useful in SPECT (single photon emission computed tomography) and in PET (positron emission tomography), as discussed further herein.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference in its entirety.

The term “pro-drug ester” refers to derivatives of the compounds disclosed herein formed by the addition of any of several ester-forming groups that are hydrolyzed under physiological conditions. Examples of pro-drug ester groups include pivoyloxymethyl, acetoxymethyl, phthalidyl, indanyl and methoxymethyl, as well as other such groups known in the art, including a (5-R-2-oxo-1,3-dioxolen-4-yl)methyl group. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is herein incorporated by reference in their entirety.

“Metabolites” of the compounds disclosed herein include active species that are produced upon introduction of the compounds into the biological milieu.

“Solvate” refers to the compound formed by the interaction of a solvent and a compound described herein, a metabolite, or salt thereof. Suitable solvates are pharmaceutically acceptable solvates including hydrates.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group of the compounds may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “haloalkyl” refers to a straight- or branched-chain alkyl group having from 1 to 12 carbon atoms in the chain, substituting one or more hydrogens with halogens. Examples of haloalkyl groups include, but are not limited to, —CF₃, —CHF₂, —CH₂F, —CH₂CF₃, —CH₂CHF₂, —CH₂CH₂F, —CH₂CH₂C₁, —CH₂CF₂CF₃ and other groups that in light of the ordinary skill in the art and the teachings provided herein, would be considered equivalent to any one of the foregoing examples.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “polyethylene glycol” refers to the formula

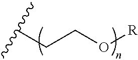

wherein n is an integer greater than one and R is a hydrogen or alkyl. The number of repeat units “n” may be indicated by referring to a number of members. Thus, for example, “2- to 5-membered polyethylene glycol” refers to n being an integer selected from two to five. In some embodiments, R is selected from methoxy, ethoxy, n-propoxy, 1-methylethoxy (isopropoxy), n-butoxy, iso-butoxy, sec-butoxy, and tert-butoxy.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. In various embodiments, a heteroaryl contains from 1 to 4 heteroatoms, from 1 to 3 heteroatoms, from 1 to 2 heteroatoms, or 1 heteroatom. For example, in various embodiments, a heteroaryl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations.

In various embodiments, a heterocyclyl contains from 1 to 4 heteroatoms, from 1 to 3 heteroatoms, from 1 to 2 heteroatoms, or 1 heteroatom. For example, in various embodiments, a heterocyclyl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆-10 aryl, 5-10 membered heteroaryl, and 5-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a “natural amino acid side chain” refers to the side-chain substituent of a naturally occurring amino acid. Naturally occurring amino acids have a substituent attached to the α-carbon. Naturally occurring amino acids include Arginine, Lysine, Aspartic acid, Glutamic acid, Glutamine, Asparagine, Histidine, Serine, Threonine, Tyrosine, Cysteine, Methionine, Tryptophan, Alanine, Isoleucine, Leucine, Phenylalanine, Valine, Proline, and Glycine.

As used herein, a “non-natural amino acid side chain” refers to the side-chain substituent of a non-naturally occurring amino acid. Non-natural amino acids include β-amino acids (β³ and β²), Homo-amino acids, Proline and Pyruvic acid derivatives, 3-substituted Alanine derivatives, Glycine derivatives, Ring-substituted Phenylalanine and Tyrosine Derivatives, Linear core amino acids and N-methyl amino acids. Exemplary non-natural amino acids are available from Sigma-Aldridge, listed under “unnatural amino acids & derivatives.” See also, Travis S. Young and Peter G. Schultz, “Beyond the Canonical 20 Amino Acids: Expanding the Genetic Lexicon,” J. Biol. Chem. 2010 285: 11039-11044, which is incorporated by reference in its entirety.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substitutents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

In some embodiments, substituted group(s) is (are) substituted with one or more substituent(s) individually and independently selected from C₁-C₄ alkyl, amino, hydroxy, and halogen.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

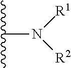

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

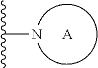

where ring A is a heterocyclyl ring containing the depicted nitrogen.

Similarly, when two “adjacent” R groups are said to form a ring “together with the atoms to which they are attached,” it is meant that the collective unit of the atoms, intervening bonds, and the two R groups are the recited ring. For example, when the following substructure is present:

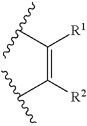

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

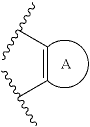

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

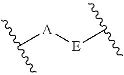

includes the substituent being oriented such that the A is attached at the leftmost attachment point of the molecule as well as the case in which A is attached at the rightmost attachment point of the molecule.

The term “agent” or “test agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved characteristics (such as higher binding affinity for a target molecule) is an approach that is well known in pharmaceutical chemistry.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rats and mice but also includes many other species.

The term “microbial infection” refers to the invasion of the host organism, whether the organism is a vertebrate, invertebrate, fish, plant, bird, or mammal, by pathogenic microbes. This includes the excessive growth of microbes that are normally present in or on the body of a mammal or other organism. More generally, a microbial infection can be any situation in which the presence of a microbial population(s) is damaging to a host mammal. Thus, a mammal is “suffering” from a microbial infection when excessive numbers of a microbial population are present in or on a mammal's body, or when the effects of the presence of a microbial population(s) is damaging the cells or other tissue of a mammal. Specifically, this description applies to a bacterial infection. Note that the compounds of preferred embodiments are also useful in treating microbial growth or contamination of cell cultures or other media, or inanimate surfaces or objects, and nothing herein should limit the preferred embodiments only to treatment of higher organisms, except when explicitly so specified in the claims.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a

### Methods of Preparation

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

In the following schemes, protecting groups for oxygen atoms are selected for their compatibility with the requisite synthetic steps as well as compatibility of the introduction and deprotection steps with the overall synthetic schemes (P. G. M. Green, T. W. Wutts, Protecting Groups in Organic Synthesis (3rd ed.) Wiley, New York (1999)).

If the compounds of the present technology contain one or more chiral centers, such compounds can be prepared or isolated as pure stereoisomers, i.e., as individual enantiomers or d(l) stereoisomers, or as stereoisomer-enriched mixtures. All such stereoisomers (and enriched mixtures) are included within the scope of the present technology, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### Synthesis of Compounds of Formula I

In one embodiment, the method involves reacting an appropriately substituted intermediate (II) with an acidic hydrogen under basic conditions with the bromo-intermediate (12F or 12G) to yield the BOC-protected derivative (III). This intermediate was then subjected to hydrolysis under acidic conditions followed by purification to yield the final product (I). (Scheme 1).

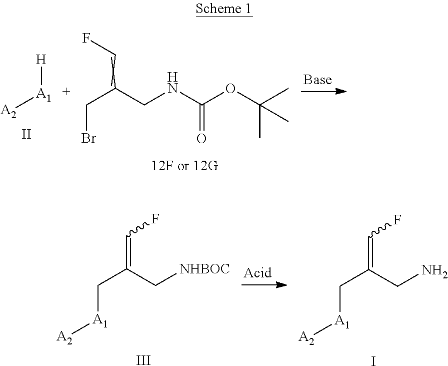

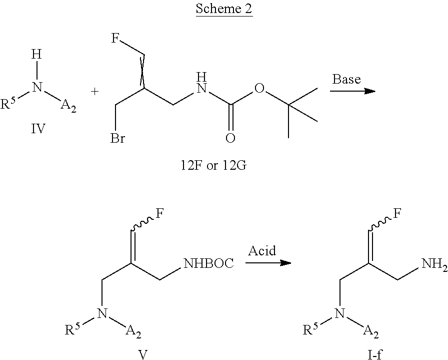

In one embodiment, the method involves reacting an appropriately substituted intermediate (IV) under basic conditions with with the bromo-intermediate (12F or 12G) to yield the BOC-protected derivative (V). This intermediate was then subjected to hydrolysis under acidic conditions followed by purification to yield the final product (I-f). (Scheme 2).

The above example schemes are provided for the guidance of the reader, and collectively represent an example method for making the compounds encompassed herein. Furthermore, other methods for preparing compounds described herein will be readily apparent to the person of ordinary skill in the art in light of the following reaction schemes and examples. Unless otherwise indicated, all variables are as defined above.

### Uses of Isotopically-Labeled Compounds

Some embodiments provide a method of using isotopically labeled compounds and prodrugs of the present disclosure in: (i) metabolic studies (preferably with ¹⁴C), reaction kinetic studies (with, for example 2H or 3H); (ii) detection or imaging techniques [such as positron emission tomography (PET) or single-photon emission computed tomography (SPECT)] including drug or substrate tissue distribution assays; or (iii) in radioactive treatment of patients.

Isotopically labeled compounds and prodrugs of the embodiments thereof can generally be prepared by carrying out the procedures disclosed in the schemes or in the examples and preparations described below by substituting a readily available isotopically labeled reagent for a non-isotopically labeled reagent. An ¹⁸F or ¹¹C labeled compound may be particularly preferred for PET, and an ¹²³I labeled compound may be particularly preferred for SPECT studies. Further substitution with heavier isotopes such as deuterium (i.e., ²H) may afford certain therapeutic advantages resulting from greater metabolic stability, for example increased in vivo half-life or reduced dosage requirements.

### Administration and Pharmaceutical Compositions

The compounds are administered at a therapeutically effective dosage. While human dosage levels have yet to be optimized for the compounds described herein, generally, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

In addition to the selected compound useful as described above, come embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

The compounds and compositions described herein, if desired, may be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compounds and compositions described herein are formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01 99.99 wt % of a compound of the present technology based on the total formulation, with the balance being one or more suitable pharmaceutical excipients. Preferably, the compound is present at a level of about 1 80 wt %. Representative pharmaceutical formulations are described below.

### FORMULATION EXAMPLES

The following are representative pharmaceutical formulations containing a compound of Formula I.

### Formulation Example 1—Tablet Formulation

The following ingredients are mixed intimately and pressed into single scored tablets.

| | Quantity per |
|---|---|
| Ingredient | tablet, mg |
| Compounds disclosed herein | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule.

| | Quantity per |
|---|---|
| Ingredient | capsule, mg |
| Compounds disclosed herein | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| Compounds disclosed herein | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |
| distilled water | q.s. to 100 | mL |

### Formulation Example 4—Injectable Formulation

The following ingredients are mixed to form an injectable formulation.

| Ingredient | Amount |
|---|---|
| Compounds disclosed herein | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of the present technology with Witepsol® H-15 (triglycerides of saturated vegetable fatty acid; Riches-Nelson, Inc., New York), and has the following composition:

| Ingredient | Amount |
|---|---|
| Compounds disclosed herein | 500 mg |
| Witepsol ® H-15 | balance |

### Methods of Treatment

The compounds disclosed herein or their tautomers and/or pharmaceutically acceptable salts thereof can effectively act as VAP-1 inhibitors and treat conditions affected at least in part by VAP-1. Some embodiments provide pharmaceutical compositions comprising one or more compounds disclosed herein and a pharmaceutically acceptable excipient. Some embodiments provide a method for treating a fibrotic disease with an effective amount of one or more compounds as disclosed herein. Some embodiments provide a method for treating a liver disorder with an effective amount of one or more compounds as disclosed herein.

In some embodiments, the subject is a human.

Further embodiments include administering a combination of compounds to a subject in need thereof. A combination can include a compound, composition, pharmaceutical composition described herein with an additional medicament.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered i.v.

Some embodiments include combinations of a compound, composition or pharmaceutical composition described herein with any other pharmaceutical compound approved for treating fibrotic disorders.

Some embodiments provide a method for inhibiting VAP-1 and/or a method for treating a disease affected at least in part by VAP-1 with an effective amount of one or more compounds as disclosed herein.

The compounds disclosed herein are useful in inhibiting VAP-1 enzyme and/or treating disorders relating to fibrosis.

The compounds disclosed herein are useful in inhibiting VAP-1 enzyme and/or treating a liver disorder.

Some embodiments provide a method for inhibiting VAP-1, which method comprises contacting cells expressing VAP-1 with an effective amount of one or more compounds as disclosed herein.

Some embodiments provide a method for treating a fibrotic disease, which method comprises administering to a subject an effective amount of one or more compounds or a pharmaceutical composition disclosed herein comprising a pharmaceutically acceptable excipient.

Some embodiments provide a method for treating a disease affected at least in part by VAP-1, which method comprises administering to a subject an effective amount of one or more compounds or a pharmaceutical composition disclosed herein comprising a pharmaceutically acceptable excipient.

Some embodiments provide a method for inhibiting VAP-1 is provided wherein the method comprises contacting cells with an effective amount of one or more compounds disclosed herein. In some embodiments a method for inhibiting VAP-1 is performed in-vitro or in-vivo.

VAP-1 is expressed in vascular endothelial cells such as high venule endothelial cells (HVE) of lymphatic organs and is also expressed in hepatic sinusoidal endothelial cells (HSEC), smooth muscle cells and adipocytes. In particular, they are important in endothelial cell surface and herein inhibition of VAP-1 also refers to inhibition in these cells as well.

### Selective Inhibition

Some embodiments provide a method for selectively inhibiting VAP-1 enzyme over Diamine Oxidase (DAO), Monoamine Oxidase A (MAO-A) and Monoamine Oxidase B (MAO-B) which includes contacting cells expressing VAP-1, DAO, MAO-A, and MAO-B respectively with an effective amount of one or more compounds disclosed herein.

Some embodiments provide a method for treating a fibrotic disease, which method comprises administering to a subject an effective amount of one or more compounds which specifically inhibits VAP-1 said compounds or a pharmaceutical composition comprising one or more compounds disclosed herein and a pharmaceutically acceptable excipient.

Some embodiments provide a method for treating a disease affected at least in part by VAP-1 which method comprises administering to a subject an effective amount of one or more compounds which specifically inhibits VAP-1, said compounds being selected from compounds disclosed herein or a pharmaceutical composition comprising one or more compounds disclosed herein and a pharmaceutically acceptable excipient.

Some embodiments provide a method for treating a fibrotic disease, which method comprises administering to a subject an effective amount of one or more compounds which selectively inhibits VAP-1, said compounds being selected from compounds disclosed herein or a pharmaceutical composition comprising one or more compounds disclosed herein and a pharmaceutically acceptable excipient.

Some embodiments provide a method for treating a disease affected at least in part by VAP-1, which method comprises administering to a subject an effective amount of one or more compounds which selectively inhibits VAP-1, said compounds being selected from compounds disclosed herein or a pharmaceutical composition comprising one or more compounds disclosed herein and a pharmaceutically acceptable excipient.

Some embodiments provide a method for treating a liver disorder, which method comprises administering to a subject an effective amount of one or more compounds which specifically inhibits VAP-1 said compounds or a pharmaceutical composition comprising one or more compounds disclosed herein and a pharmaceutically acceptable excipient.

Some embodiments provide a method for treating a liver disorder, which method comprises administering to a subject an effective amount of one or more compounds which selectively inhibits VAP-1, said compounds being selected from compounds disclosed herein or a pharmaceutical composition comprising one or more compounds disclosed herein and a pharmaceutically acceptable excipient.

Some embodiments provide a method for prophylactic therapy or treatment of a subject having a fibrotic disorder wherein said method comprising administering an effective amount of one or more compounds disclosed herein to the subject in need thereof.

Some embodiments provide a method for prophylactic therapy or treatment of a subject having a disorder affected by VAP-1 wherein said method comprising administering an effective amount of one or more compounds disclosed herein to the subject in need thereof.

Some embodiments provide a method for inhibiting myofibroblast differentiation (e.g., Epithelial/Endothelial-to-Mesenchymal Transition (EpMT/EnMT)) is provided wherein the method comprises contacting cells with an effective amount of one or more compounds disclosed herein. In one aspect, the method for inhibiting myofibroblast differentiation (e.g., Epithelial/Endothelial-to-Mesenchymal Transition (EpMT/EnMT)) is performed in-vitro or in-vivo.

Some embodiments provide a method for treating a disease or condition selected from the group consisting of or that produces a symptom selected from the group consisting of: progressive liver fibrosis, renal fibrosis, idiopathic lung fibrosis, diabetic nephropathy, systemic sclerosis, idiopathic pulmonary fibrosis, non-alcoholic steatohepatitis, primary sclerosing cholangitis, corneal fibrosis, liver cirrhosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis diseases, wherein which method comprises administering to a subject an effective amount of one or more compounds disclosed herein to a subject in need thereof.

Some embodiments provide a method for treating progressive liver fibrosis.

Some embodiments provide a method for treating non-alcoholic steatohepatitis.

Some embodiments provide a method for treating fibrosis in rheumatoid arthritis diseases.

Some embodiments provide a method for treating a condition affected by VAP-1, which is in both a therapeutic and prophylactic setting for subjects. Both methods comprise administering of one or more compounds disclosed herein to a subject in need thereof.

Some embodiments provide a method for treating stiff skin syndrome.

Preferred embodiments include combinations of a compound, composition or pharmaceutical composition described herein with other VAP-1 inhibitor agents, such as anti-VAP-1 antibodies or antibody fragments, VAP-1 antisense, iRNA, or other small molecule VAP-1 inhibitors.

Some embodiments include combinations of a compound, composition or pharmaceutical composition described herein to inhibit myofibroblast differentiation (e.g., Epithelial/Endothelial-to-Mesenchymal Transition (EpMT/EnMT)).

Some embodiments include combinations of one or more of these compounds which are inhibitors of VAP-1, alone or in combination with other TGFβ signaling inhibitors, could be used to treat or protect against or reduce a symptom of a fibrotic, sclerotic or post inflammatory disease or condition including: liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, postvasectomy pain syndrome, and rheumatoid arthritis.

Some embodiments include a combination of the compounds, compositions and/or pharmaceutical compositions described herein with an additional agent, such as anti-inflammatories including glucocorticoids, analgesics (e.g. ibuprofen), aspirin, and agents that modulate a Th2-immune response, immunosuppressants including methotrexate, mycophenolate, cyclophosphamide, cyclosporine, thalidomide, pomalidomide, leflunomide, hydroxychloroquine, azathioprine, soluble bovine cartilage, vasodilators including endothelin receptor antagonists, prostacyclin analogues, nifedipine, and sildenafil, IL-6 receptor antagonists, selective and non-selective tyrosine kinase inhibitors, Wnt-pathway modulators, PPAR activators, caspase-3 inhibitors, LPA receptor antagonists, B cell depleting agents, CCR2 antagonists, pirfenidone, cannabinoid receptor agonists, ROCK inhibitors, miRNA-targeting agents, toll-like receptor antagonists, CTGF-targeting agents, NADPH oxidase inhibitors, tryptase inhibitors, TGFD inhibitors, relaxin receptor agonists, and autologous adipose derived regenerative cells.

### Indications

In some embodiments, the compounds and compositions comprising the compounds described herein can be used to treat a host of conditions arising from fibrosis or inflammation, and specifically including those associated with myofibroblast differentiation. Example conditions include progressive liver fibrosis (alcoholic, viral, autoimmune, metabolic and hereditary chronic disease), renal fibrosis (e.g., resulting from chronic inflammation, infections or type II diabetes), lung fibrosis (idiopathic or resulting from environmental insults including toxic particles, sarcoidosis, asbestosis, hypersensitivity pneumonitis, bacterial infections including tuberculosis, medicines, etc.), interstitial fibrosis, systemic scleroderma (autoimmune disease in which many organs become fibrotic), macular degeneration (fibrotic disease of the eye), pancreatic fibrosis (resulting from, for example, alcohol abuse and chronic inflammatory disease of the pancreas), fibrosis of the spleen (from sickle cell anemia, other blood disorders), cardiac fibrosis (resulting from infection, inflammation and hypertrophy), mediastinal fibrosis, myelofibrosis, endomyocardial fibrosis, retroperitoneal fibrosis, progressive massive fibrosis, nephrogenic systemic fibrosis, diabetic nephropathy, non-alcoholic steatohepatitis, primary sclerosing cholangitis, corneal fibrosis, liver cirrhosis, fibrotic complications of surgery, chronic allograft vasculopathy and/or chronic rejection in transplanted organs, ischemic reperfusion injury associated fibrosis, injection fibrosis, cirrhosis, diffuse parenchymal lung disease, post-vasectomy pain syndrome, and rheumatoid arthritis diseases or disorders.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples. The following examples will further describe the present invention, and are used for the purposes of illustration only, and should not be considered as limiting.

### Examples

### General Procedures

It will be apparent to the skilled artisan that methods for preparing precursors and functionality related to the compounds claimed herein are generally described in the literature. In these reactions, it is also possible to make use of variants which are themselves known to those of ordinary skill in this art, but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March Advanced Organic Chemistry (Wiley), Carey and Sundberg, Advanced Organic Chemistry (incorporated herein by reference in their entirety) and the like. All the intermediate compounds of the present invention were used without further purification unless otherwise specified.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

The following example schemes are provided for the guidance of the reader, and represent preferred methods for making the compounds exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

The following abbreviations have the indicated meanings:
ACN=Acetonitrile
DCM=dichloromethane
DIEA=N,N-Diisopropylethylamine
DIPEA=N,N-Diisopropylethylamine
DMF=N,N-dimethylformamide
DMP=Dess Martin Periodinane
DNs=dinitrosulfonyl
ESBL=extended-spectrum β-lactamase
EtOAc=ethyl acetate
EA=ethyl acetate
FCC=Flash Column Chromatography
HATU=2-(7-aza-1H-benzotriazole-1-yl)-1,1,3,3-tetramethyluronium hexafluorophosphate
MeCN=acetonitrile
NMR=nuclear magnetic resonance
PE=Petroleum Ether
Prep=preparatory
Py=pyridine
Sat.=saturated aqueous
TBDMSCl=tert-butyldimethylsilyl chloride
TBS=tert-butyldimethylsilyl
TFA=trifluoroacetic acid
THF=tetrahydrofuran
TLC=thin layer chromatography
TrtCl=Trityl chloride
Trt=Trityl

The following example schemes are provided for the guidance of the reader, and collectively represent an example method for making the compounds provided herein. Furthermore, other methods for preparing compounds described herein will be readily apparent to the person of ordinary skill in the art in light of the following reaction schemes and examples. Unless otherwise indicated, all variables are as defined above.

### Example 1

### Compound 12

### 3-fluoro-2-(piperidin-1-ylmethyl)prop-2-en-1-amine (12)

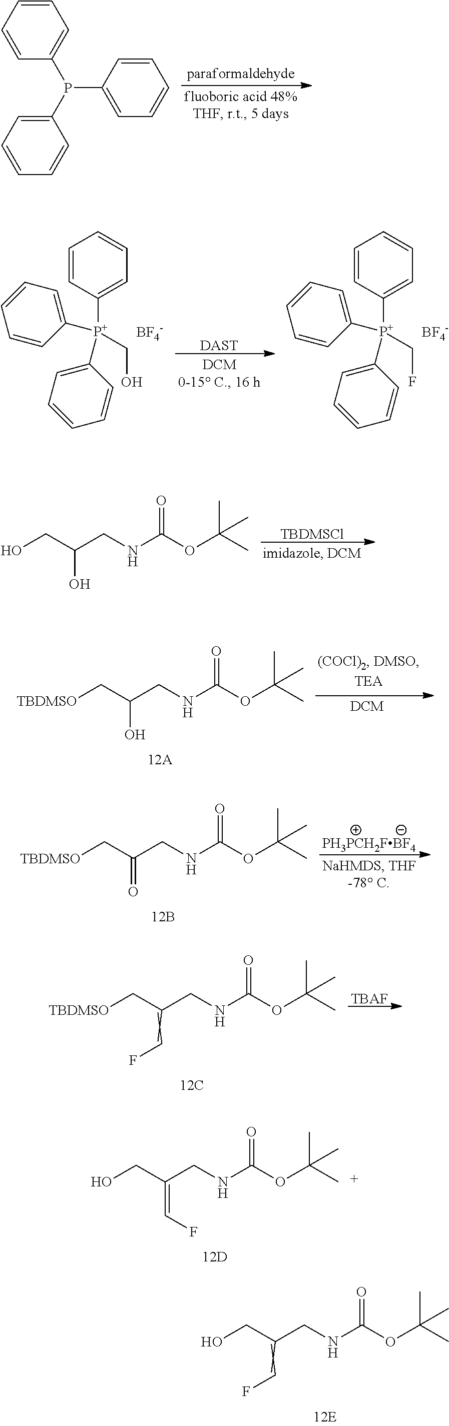

Into a one liter one-necked flask, fitted with a stir bar and a water-cooled reflux condenser, was charged PPh₃ (131.3 g, 500.60 mmol), paraformaldehyde (15.03 g, 500.60 mmol) and THF (500 mL). The solution was moderately stirred as HBF₄ aqueous (164 mL, 1.26 mol, 48%) was slowly added. An exothermic reaction ensued and a large quantity of precipitate formed. After the addition of the HBF₄ was completed, the reaction mixture was stirred at room temperature (5-20° C.) for five days. The solvent was removed under reduced pressure to give a residue. The residue was triturated in H₂O (300 mL), filtered and the filter cake was triturated in TBME (300 mL), filtered and the filter cake was dried under reduced pressure. Compound (hydroxymethyl)triphenylphosphonium tetrafluoroborate (184 g, yield: 96.7%, BF₄) was obtained as a white solid. ¹H NMR (400 MHz, CDCl₃) δ 7.80-7.62 (m, 15H), 5.23 (s, 2H), 4.48 (br. s, 1H).

To the mixture of (hydroxymethyl)triphenylphosphonium tetrafluoroborate (184 g, 484.05 mmol) in DCM (85 mL), DAST (64 mL, 484.05 mmol) was added dropwise at 0° C. and then the reaction mixture was stirred at room temperature (5-15° C.) overnight (16 h). The reaction mixture was diluted with ice H₂O (960 mL) and separated, the organic phase was dried over Na₂SO₄ and filtered. The filtrate was mixed with TBME (300 mL) to recrystallize the product. After overnight, more crystals precipitated out, filtered and the filter cake was dried under reduced pressure. Compound (fluoromethyl)triphenylphosphonium tetrafluoroborate (108 g, yield: 58.39%, BF₄) was obtained as a light yellow solid. ¹H NMR (400 MHz, DMSO-d₆) δ 7.87-7.70 (m, 15H), 6.29 (d, J=45.2 Hz, 2H).

To a solution of tert-butyl (2,3-dihydroxypropyl)carbamate (15 g, 78.44 mmol) in DCM (200 mL) was added imidazole (10.68 g, 156.88 mmol) and TBDMSCl (82.93 mmol, 10.16 mL) at 0° C., the mixture was stirred at 15° C. for 2 h. The reaction mixture was diluted with water (300 mL), extracted with DCM (100 mL×2), the organic layers were washed with brine (200 mL), dried over Na₂SO₄, filtered and concentrated to give a residue. The residue was purified by flash silica gel chromatography (ISCO; 220 g SepaFlash© Silica Flash Column, Eluent of 0 20% Ethyl acetate/Petroleum ether gradient @ 100 mL/min). Compound 12A (21 g, yield: 87.6%) as colorless oil was obtained. ¹H NMR (400 MHz, CDCl₃) δ 4.89 (s, 1H), 3.66 (s, 1H), 3.57 (dd, J=4.5, 10.1 Hz, 1H), 3.46 (dd, J=6.1, 10.0 Hz, 1H), 3.28 (d, J=11.2 Hz, 1H), 3.10-3.01 (m, 1H), 2.77 (s, 1H), 1.37 (s, 9H), 0.86-0.80 (m, 9H), 0.01 (s, 6H).

To a solution of (COCl)₂ (103.11 mmol, 9.03 mL) in DCM (250 mL) at −78° C. under N2 was added DMSO (161.26 mmol, 12.60 mL) in DCM (30 mL) dropwise over 30 min. After addition, the solution was stirred at −78° C. for 1 h. A solution of compound 33A (21 g, 68.74 mmol) in DCM (80 mL) was then added dropwise over 30 min, the solution was stirred at −78° C. for 1 h, then TEA (301.75 mmol, 42.00 mL) was added, the mixture was allowed to warm to 10° C. and stirred 1 h. The reaction mixture was partitioned between water (200 mL) and DCM (50 mL), the aqueous layer was extracted with DCM (80 mL×2), and the organic layers were washed with brine (150 mL), dried over Na₂SO₄, filtered and concentrated to give a residue. The residue was purified by flash silica gel chromatography (ISCO; 220 g SepaFlash® Silica Flash Column, Eluent of 0˜10% Ethyl acetate/Petroleum ether gradient @ 100 mL/min). Compound 12B (18.5 g, yield: 88.7%) as colorless oil was obtained. ¹H NMR (400 MHz, CDCl₃) δ 5.20 (br s, 1H), 4.29-4.20 (m, 4H), 1.45 (s, 9H), 0.92 (s, 9H), 0.13-0.05 (m, 6H).

To a suspension of (fluoromethyl)triphenylphosphonium tetrafluoroborate (32.74 g, 85.68 mmol, BF₄) in THF (200 mL) at −78° C. under N2 was added NaHMDS (1M, 85.68 mL) over 30 min, the mixture was stirred for 1 h at −78° C. A solution of compound 12B (13 g, 42.84 mmol) in THF (50 mL) was added slowly. After addition, the mixture was stirred for 20 h at −78° C. The reaction mixture was quenched by water (300 mL), concentrated to remove THF, then extracted with TBME (100 mL×3), the organic layers were washed with brine (100 mL), dried over Na₂SO₄, filtered and concentrated to give a residue. The residue was purified by flash silica gel chromatography (ISCO; 120 g SepaFlash® Silica Flash Column, Eluent of 0˜10% Ethyl acetate/Petroleum ether gradient @ 85 mL/min). Compound 12C (10.5 g, yield: 76.7%) as light yellow oil was obtained. ¹H NMR (400 MHz, CDCl₃) δ 6.66-6.39 (m, 1H), 4.82 (s, 1H), 4.02 (dd, J=1.2, 4.4 Hz, 2H), 3.82 (d, J=3.4 Hz, 2H), 1.36 (s, 9H), 0.82 (s, 9H), 0.01 (s, 6H).

To a solution of compound 12C (11 g, 34.43 mmol) in THF (150 mL) was added TBAF (1M, 40 mL), the mixture was stirred at 13° C. for 1 h. The reaction mixture was diluted with water (200 mL), extracted with EA (80 mL×3), the organic layers were washed with water (200 mL×2) and brine (200 mL×2), dried over Na₂SO₄, filtered and concentrated to give a residue. The residue was purified by flash silica gel chromatography (ISCO®; 120 g SepaFlash® Silica Flash Column, Eluent of 0 20% Ethyl acetate/Petroleum ether gradient @ 85 mL/min). Compound 12E (0.9 g, yield: 12.7%) as light yellow oil was obtained. (A mixture of Z/E=1:1). Compound 12D (5.1 g, yield: 72.2%) as light yellow oil was obtained. ¹H NMR (400 MHz, CDCl₃) δ 6.72 (s, 1H), 6.61 (s, 1H), 6.51 (s, 1H), 6.41 (s, 1H), 4.89 (s, 1H), 3.99-3.90 (m, 4H), 1.45 (s, 9H)

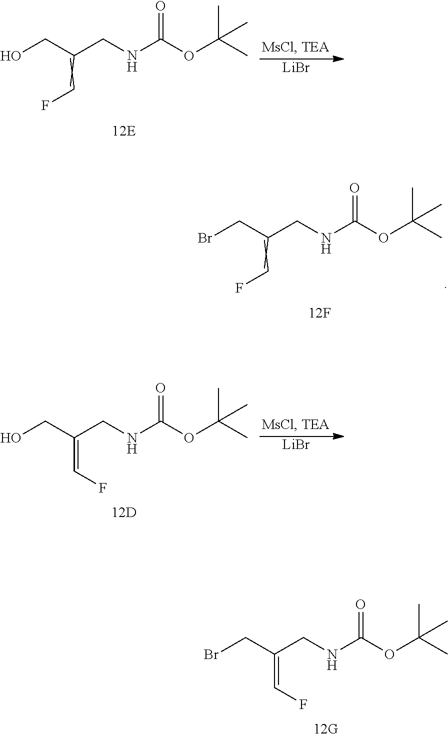

To a solution of compound 12E (1.1 g, 5.36 mmol) in acetone (20 mL) was added TEA (8.04 mmol, 1.12 mL), then MsCl (8.73 mmol, 0.68 mL) was added under N2 at 0° C., the mixture was stirred at this temperature for 30 min, the mixture was filtered to remove the precipitated salts and the filter cake was further washed with acetone (10 mL), the filtrate was added LiBr (2.33 g, 26.80 mmol), the suspension was further stirred at 15° C. for 1 h. The reaction mixture was partitioned between water (50 mL) and ethyl acetate (30 mL) and the aqueous layer was further extracted with ethyl acetate (30 mL×2). The combined organics were washed with brine (50 mL), dried over Na₂SO₄, filtered and concentrated in vacuo to give a residue. Compound 12F (1.2 g, yield: 83.5%) as light yellow oil was obtained, which was used into the next step without further purification. ¹H NMR (400 MHz, CDCl₃) δ 6.87 (s, 1H), 6.73 (s, 1H), 6.67 (s, 1H), 6.53 (s, 1H), 4.74 (s, 1H), 4.07 (d, J=2.7 Hz, 2H), 4.00 (d, J=5.1 Hz, 2H), 3.97-3.92 (m, 2H), 3.78 (s, 2H), 1.45 (s, 18H).

To a solution of compound 12D (6.6 g, 32.16 mmol) in acetone (60 mL) was added TEA (50.29 mmol, 7 mL), then MsCl (46.70 mmol, 3.61 mL) was added under N2 at 0° C., the mixture was stirred at this temperature for 30 min, the mixture was filtered to remove the precipitated salts and the filter cake was further washed with acetone (40 mL). and then the filtrate was added LiBr (14 g, 161.21 mmol), the suspension was further stirred at 15° C. for 1 h. The reaction mixture was partitioned between water (200 mL) and ethyl acetate (80 mL), the aqueous layer was further extracted with ethyl acetate (50 mL×2). The combined organics were washed with brine (100 mL), dried over Na₂SO₄, filtered and concentrated in vacuo to give a residue. Compound 12G (8.8 g, crude) as light yellow oil was obtained, which was used into the next step without further purification. ¹H NMR (400 MHz, CDCl₃) δ 6.87 (s, 1H), 6.66 (s, 1H), 4.77 (s, 1H), 4.01-3.94 (m, 4H), 1.44 (s, 9H).

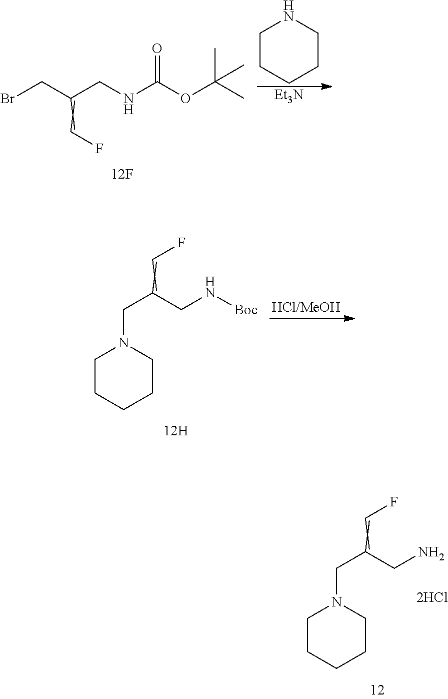

To a solution of compound 12F (120 mg, 447.56 umol) in DMF (2 mL) was added Et₃N (145.40 mg, 1.44 mmol, 0.2 mL) and piperidine (76.22 mg, 895.11 umol, 88.40 uL). The mixture was stirred at 20° C. for 3 h. The reaction mixture was quenched with H₂O (2 mL) and partitioned between EtOAc (20 mL) and H₂O (20 mL). The organic phase was separated, washed with brine (20 mL), dried over Na₂SO₄, filtered and concentrated under reduced pressure to give a residue. Compound 12H (100 mg, yield: 82.0%) was obtained as a yellow oil. ¹H NMR (400 MHz, CDCl₃) δ 6.44 (d, J=84 Hz, 1H), 6.20 (br s, 1H), 3.91 (br s, 2H), 2.85-2.74 (m, 2H), 2.33 (br s, 4H), 1.62-1.50 (m, 5H), 1.44 (s, 10H). MS (ESI) m/z (M+H)⁺ 273.2.

To a solution of compound 12H (100 mg, 367.16 umol) in MeOH (2 mL) was added MeOH/HCl (4M, 4 mL). The mixture was stirred at 15° C. for 5 h. The reaction was concentrated to give a residue. The residue was purified by preparatory-HPLC (HCl condition; column: YMC-Actus Triart C18 100*30 mm*5 um; mobile phase: [water (0.05% HC)-ACN]; B %: 0%-5%, 4.5 min). Compound 12 (70 mg, yield: 75.9%, 2HCl, mixture of E/Z, ratio: 7:1) was obtained as a white solid. ¹H NMR (400 MHz, DMSO-d₆) δ 10.59 (br s, 1H), 8.52 (br s, 3H), 7.25 (d, J=81.2 Hz, 1H), 3.89-3.86 (m, 2H), 3.80 (br d, J=4.4 Hz, 2H), 3.42-3.31 (m, 2H), 2.94-2.73 (m, 2H), 1.91-1.65 (m, 5H), 1.43-1.24 (m, 1H). MS (ESI) m/z (M+H)+173.2.

### Example 2

### Compounds 5-6

### (E)-2-((benzyloxy)methyl)-3-fluoroprop-2-en-1-amine hydrochloride (5)

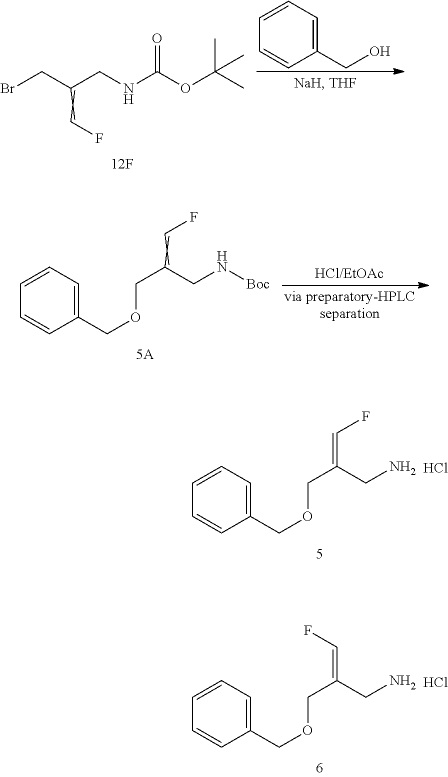

To a suspension of NaH (25 mg, 625.06 umol, 60% purity) in dry THF (3 mL) was added via cannula a solution of phenylmethanol (60 mg, 554.85 umol, 57.69 uL) in dry THF (0.5 mL) at 0° C. The reaction was stirred at 0° C. for 30 min under an inert atmosphere. Compound 12F (150 mg, 559.44 umol) in dry THF (0.5 mL) was then added dropwise. The resulting suspension was stirred for 60 min at 20° C. The solvent was quenched with saturated ammonium chloride solution (2 mL) and H₂O (20 mL) was added. The aqueous phase was extracted with ethyl acetate (3×15 mL) and the combined organic extracts were dried over Na₂SO₄, filtered and concentrated in vacuo. The residue was purified preparatory-TLC (SiO₂, PE:EA=3:1). Compound 5A (120 mg, 57.1% yield) was obtained as a yellow oil. ¹H NMR (400 MHz, CDCl₃) δ 7.38-7.27 (m, 5H), 6.61 (d, J=83.2 Hz, 1H), 4.83 (br s, 1H), 4.48 (s, 2H), 4.01-3.84 (m, 4H), 1.53-1.37 (m, 9H). MS (ESI) m/z (M-Boc+H)⁺ 196.1.

To a solution of compound 5A (120 mg, 406.30 umol) in EA (2 mL) was added EA/HCl (4M, 6 mL). The mixture was stirred at 12° C. for 5 h. The reaction was concentrated. The residue was purified by preparatory-HPLC (HCl condition; column: YMC-Actus Triart C18 100*30 mm*5 um; mobile phase: [water (0.05% HCl)-ACN]; B %: 0%-40%, 10 min). Compound 5 (60 mg, yield: 63.7%, HCl) was obtained as a white solid. ¹H NMR (400 MHz, DMSO-d₆) δ 8.35 (br s, 2H), 7.41-7.33 (m, 4H), 7.33-7.27 (m, 1H), 7.15 (d, J=82.4 Hz, 1H), 4.47 (s, 2H), 4.08 (d, J=3.4 Hz, 2H), 3.51 (br s, 2H). MS (ESI) m/z (M+H)⁺ 196.1.

### (Z)-2-((benzyloxy)methyl)-3-fluoroprop-2-en-1-amine hydrochloride (6)

Compound 6 (15 mg, yield: 15.3%, HCl) was obtained as a white solid. ¹H NMR (400 MHz, Methanol-d₄) δ 7.40-7.26 (m, 5H), 6.98 (d, J=81.6 Hz, 1H), 4.58-4.52 (m, 2H), 4.26 (dd, J=1.0, 2.9 Hz, 2H), 3.59 (d, J=1.7 Hz, 2H). MS (ESI) m/z (M+H)⁺ 196.1.

### Example 3

### Compounds 7,18, 23-24, and 55-56

### (E)-1-(4-((2-(aminomethyl)-3-fluoroalkyl)oxy)phenyl)-3-ethylurea hydrochloride (7)

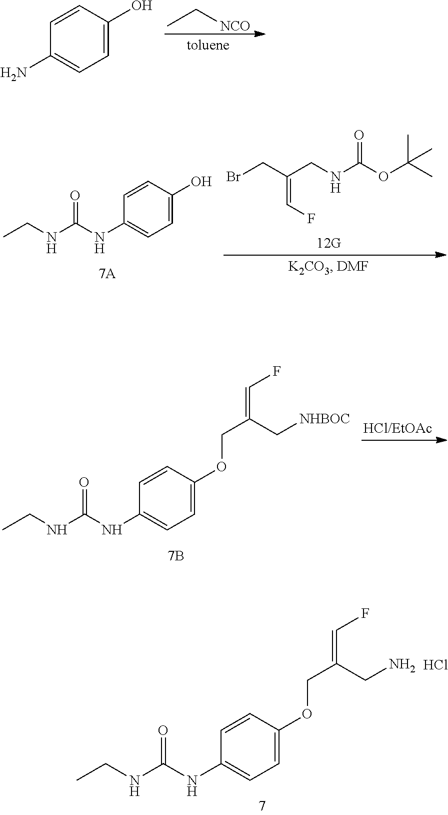

To a mixture of 4-aminophenol (1 g, 9.16 mmol, 1.43 mL) in toluene (30 mL) was added isocyanatoethane (716.47 mg, 10.08 mmol, 797.85 uL) at 0° C. The mixture was stirred at 15° C. for 16 hr. The reaction was diluted with H₂O (30 mL), and extracted with EA (20 mL×3), dried over Na₂SO₄, filtered and concentrated. The residue was purified by flash silica gel chromatography (ISCO©; 12 g SepaFlash© Silica Flash Column, Eluent of 70% Ethyl acetate/Petroleum ether gradient @ 30 mL/min). Compound 7A (300 mg, yield: 18.2%) was obtained as a yellow solid. ¹H NMR (400 MHz, DMSO-d₆) δ 8.89 (s, 1H), 7.99 (s, 1H), 7.18-7.03 (m, 2H), 6.66-6.49 (m, 2H), 5.87 (t, J=5.6 Hz, 1H), 3.08-2.98 (m, 2H), 0.99 (t, J=7.2 Hz, 3H). MS (ESI) m/z (M+H)⁺ 181.1.

To a solution of compound 12G (200 mg, 745.93 umol) and compound 7A (150 mg, 832.39 umol) in DMF (5 mL) was added K₂CO₃ (155 mg, 1.12 mmol). The mixture was stirred at 15° C. for 16 h. The reaction was diluted with H₂O (30 mL), extracted with EA (20 mL×2), the organic phase was washed with 1N NaOH (20 mL), and brine (20 mL), and dried over Na₂SO₄, filtered and concentrated. The residue was used to the next step without purification compound 7B (250 mg, yield: 91.2%) was obtained as a yellow solid. ¹H NMR (400 MHz, DMSO-d₆) δ 8.35 (br s, 2H), 7.41-7.33 (m, 4H), 7.33-7.27 (m, 1H), 7.15 (d, J=82.4 Hz, 1H), 4.47 (s, 2H), 4.08 (d, J=3.4 Hz, 2H), 3.51 (br s, 2H). MS (ESI) m/z (M-Boc+H)⁺ 268.2.

To a solution of compound 7B (250 mg, 680.43 umol) in EA (1 mL) was added HCl/EtOAc (4M, 6 mL). The mixture was stirred at 20° C. for 2 h. The reaction was concentrated to give a residue. The residue was purified by preparatory-HPLC (HCl condition; column: YMC-Actus Triart C18 100*30 mm*5 um; mobile phase: [water (0.05% HCl)-ACN]; B %: 0%-25%, 8.5 min). Compound 7 (150 mg, yield: 72.5%, HCl) was obtained as a gray solid. ¹H NMR (400 MHz, DMSO-d₆) δ 8.61 (br s, 1H), 8.29 (br s, 3H), 7.38-7.10 (m, 3H), 6.85 (d, J=9.0 Hz, 2H), 4.54 (br d, J=3.4 Hz, 2H), 3.55 (br d, J=4.6 Hz, 2H), 3.05 (q, J=7.3 Hz, 2H), 1.01 (t, J=7.2 Hz, 3H). MS (ESI) m/z (M+H)⁺ 268.2.

### (E)-1-(4-((2-(aminomethyl)-3-fluoroalkyl)oxy)phenyl)-3-isopropylurea hydrochloride (18)

4-Aminophenol and 2-isocyanatopropane were subjected to same conditions as for intermediate 7A and then the resulting intermediate was treated with intermediate 12G under conditions as described for compound 7 to yield compound 18. Compound 18 (115 mg, yield 48.7%) was obtained as an off-white solid was obtained: ¹H NMR (400 MHz, DMSO-d₆) δ 8.44 (s, 1H), 8.23 (br. s., 3H), 7.29 (d, J=8.4 Hz, 2H), 7.27 (d, J=82.0 Hz, 1H), 6.86 (d, J=8.8 Hz, 2H), 6.11 (d, J=7.6 Hz, 1H), 4.54 (d, J=2.8 Hz, 2H), 3.77-3.67 (m, 1H), 3.58 (br. s., 2H), 1.07 (d, J=6.4 Hz, 6H). ¹⁹F NMR (376 MHz, DMSO-d₆) δ−123.63. MS (ESI) m/z (M+H)⁺ 282.2.

### (E)-1-(4-((2-(aminomethyl)-3-fluoroalkyl)oxy)phenyl)-3-ethyl-1-methylurea hydrochloride (23)

4-(methylamino)phenol and isocyanatoethane were subjected to same conditions as for intermediate 7A and then the resulting intermediate was treated with intermediate 12G under conditions as described for compound 7 to yield compound 23. Compound 23 (70 mg, yield 36.4%) was obtained as a brown solid was obtained: ¹H NMR (400 MHz, DMSO-d₆) δ 8.48 (br. s., 3H), 7.30 (d, J=80.0 Hz, 1H), 7.16 (d, J=8.8 Hz, 2H), 6.99 (d, J=8.8 Hz, 2H), 4.66 (d, J=3.2 Hz, 2H), 3.56 (d, J=4.4 Hz, 2H), 3.07 (s, 3H), 3.04-2.95 (m, 2H), 0.99-0.90 (m, 3H). ¹⁹F NMR (376 MHz, DMSO-d₆) δ−123.57 (s, 1F). MS (ESI) m/z (M+H)⁺ 282.1.

### (E)-3-(4-((2-(aminomethyl)-3-fluoroalkyl)oxy)phenyl)-1,1-diethylurea hydrochloride (24)

4-Aminophenol and diethylcarbamic chloride were subjected to treatment with DCM and TEA at 20° C. for 12 h and the resulting intermediate was treated with intermediate 12G under conditions as described for compound 7 to yield compound 24. Compound 24 (70 mg, yield 36.4%) was obtained as an off-white solid was obtained: ¹H NMR (400 MHz, DMSO-d₆) δ 8.35 (br. s., 3H), 8.06 (br. s., 1H), 7.39-7.36 (m, 2H), 7.27 (d, J=82.0 Hz, 1H), 6.87 (d, J=9.2 Hz, 2H), 4.57 (d, J=3.2 Hz, 2H), 3.56 (d, J=5.2 Hz, 2H), 3.37-3.26 (m, 4H), 1.06 (t, J=6.8 Hz, 6H). ¹⁹F NMR (376 MHz, DMSO-d₆) δ−123.71. MS (ESI) m/z (M+H)⁺ 296.1.

### (E)-4-((2-(aminomethyl)-3-fluoroalkyl)oxy)phenyl ethylcarbamate (55)

Hydroquinone and isocyanatoethane were subjected to treatment with TEA in THF and TEA at 20° C. for 1.6 h and the resulting intermediate was treated with intermediate 12F under conditions as described for compound 7 to yield mixture of compounds 55 and 56. The product was purified by preparatory-HPLC (water (0.05% HCl)-ACN). Compound 55 (40 mg, yield 23.45%) was obtained as a yellow solid was obtained: ¹H NMR (400 MHz, DMSO-d₆): δ 8.23 (br s, 3H), 8.05 (d, J=8.8 Hz, 2H), 7.48-7.26 (m, 1H), 7.48-7.26 (m, 1H), 7.23 (d, J=9.0 Hz, 2H), 4.73 (d, J=3.0 Hz, 2H), 3.63 (br s, 2H). MS (ESI) m/z (M+H)⁺ 269.2.

### (Z)-4-((2-(aminomethyl)-3-fluoroalkyl)oxy)phenyl ethylcarbamate (56)

Compound 56 (8.5 mg, yield 4.97%) was obtained as a yellow solid was obtained: ¹H NMR (CD₃OD, 400 MHz): δ 7.20-6.98 (m, 5H), 7.10 (s, 1H), 4.80 (d, J=2.3 Hz, 2H), 3.68 (d, J=1.8 Hz, 2H), 3.20 (q, J=7.3 Hz, 2H), 1.16 (t, J=7.3 Hz, 3H). MS (ESI) m/z (M+H)⁺ 269.1.

### Example 4

### Compound 1

### (E)-N-(2-(aminomethyl)-3-fluoroalkyl)-N-phenylacetamide hydrochloride (1)

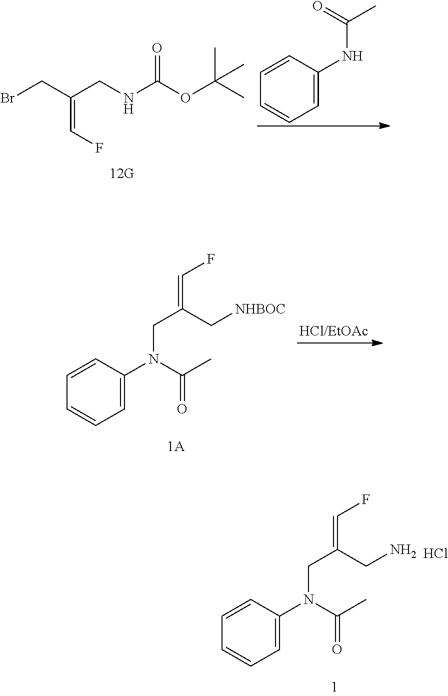

To a solution of N-phenylacetamide (90 mg, 665.86 umol) in THF (8 mL) was added NaH (29 mg, 727.28 umol, 60% purity) at 0° C., then intermediate 12G (150 mg, 559.45 umol) was added, the mixture was stirred at 15° C. for 1 h. The reaction mixture was diluted with water (50 mL), extracted with EA (30 mL×2), the organic layers were washed with water (50 mL) and brine (50 mL), dried over Na₂SO₄, filtered and concentrated to give a residue. The residue was purified by preparatory-TLC (plate 1, PE:EA=2:1). Compound 1 (100 mg, yield: 55.4%) as colorless oil was obtained. ¹H NMR (400 MHz, CDCl₃) δ 7.47-7.33 (m, 3H), 7.10 (d, J=7.6 Hz, 2H), 6.34-6.00 (m, 1H), 5.72 (br s, 1H), 4.20 (d, J=2.7 Hz, 2H), 3.90 (d, J=6.1 Hz, 2H), 1.87 (s, 3H), 1.47-1.42 (m, 9H). MS (ESI) m/z (M-Boc+H)⁺ 223.2.

To a solution of compound 1A (100 mg, 310.20 umol) in EA (2 mL) was added HCl/EtOAc (4M, 4 mL), then the mixture was stirred at 15° C. for 1 h. The reaction mixture was concentrated to give a residue. The residue was purified by preparatory-HPLC (column: YMC—Actus Triart C18 100*30 mm*5 um; mobile phase: [water (0.05% HCl)-ACN]; B %: 0%-48%, 8 min). Compound 1 (35 mg, yield: 43.4%, HCl) as white sticky compound was obtained. ¹H NMR (400 MHz, DMSO-d₆) δ 8.33 (br s, 3H), 7.52-7.26 (m, 5H), 6.90-6.55 (m, 1H), 4.31 (br s, 2H), 3.48 (br d, J=5.0 Hz, 2H), 1.78 (s, 3H). MS (ESI) m/z (M+H)⁺ 223.0.

### Example 5

### Compounds 2-4, 8-11, 13, and 82

### (E)-N-(2-(aminomethyl)-3-fluoroalkyl)-N-phenylacetamide hydrochloride (2)

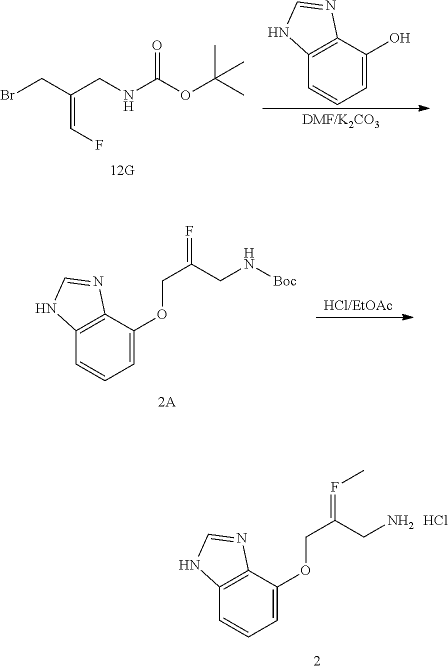

To a solution of intermediate 12G (250 mg, 932.41 umol) in DMF (8 mL) was added K₂CO₃ (190 mg, 1.37 mmol) and 1H-benzimidazol-4-ol (150 mg, 1.12 mmol), the mixture was stirred at 20° C. for 3 h. The reaction mixture was diluted with water (50 mL), extracted with EA (20 mL×3), the organic layers were washed with water (50 mL) and brine (50 mL), dried over Na₂SO₄, filtered and concentrated to give a residue. The residue was purified by preparatory-TLC (plate 1, DCM:MeOH=15:1). Compound 2A (80 mg, yield: 24.0%) as yellow oil was obtained. ¹H NMR (400 MHz, CDCl₃) δ 8.04 (br s, 1H), 7.41 (br d, J=7.3 Hz, 1H), 7.21-7.12 (m, 1H), 6.93-6.59 (m, 2H), 5.20 (br s, 1H), 4.61 (br s, 2H), 4.16-4.03 (m, 2H), 1.47-1.40 (m, 9H). MS (ESI) m/z (M+H)⁺ 322.2.

To a solution of compound 2A (90 mg, 280.07 umol) in EA (2 mL) was added HCl/EtOAc (4M, 4 mL), the mixture was stirred at 15° C. for 1 h. The reaction mixture was concentrated to give a residue. The residue was purified by preparatory-HPLC (column: YMC-Actus Triart C18 100*30 mm*5 um; mobile phase: [water (0.05% HCl)—ACN]; B %: 0%-5%, 4.5 min). Compound 2 (13 mg, yield: 18.0%, HCl) as colorless sticky compound was obtained. ¹H NMR (400 MHz, DMSO-d₆) δ=9.63 (s, 1H), 8.54 (br s, 3H), 7.57 (s, 1H), 7.54-7.46 (m, 1H), 7.46-7.39 (m, 1H), 7.17 (d, J=8.1 Hz, 1H), 4.91 (br d, J=2.9 Hz, 2H), 3.74 (br d, J=4.9 Hz, 2H). MS (ESI) m/z (M+H)⁺ 222.1.

### (E)-2-(fluoromethylene)-N¹-phenylpropane-1,3-diamine hydrochloride (3)

Intermediate 12G and tert-butyl phenylcarbamate were subjected to conditions as described for compound 2 to yield compound 3. Compound 3 (37 mg, yield: 39.3%) as a yellow solid was obtained: ¹H NMR (400 MHz, DMSO-d₆) δ 8.42 (br s, 3H), 7.31-7.20 (m, 2H), 7.01 (br s, 2H), 6.95-6.87 (m, 1H), 3.93 (br d, J=2.7 Hz, 2H), 3.60 (br s, 2H). MS (ESI) m/z (M+H)⁺ 181.2.

### (E)-2-((4-(1H-imidazol-2-yl)phenoxy)methyl)-3-fluoroprop-2-en-1-amine (4)

Intermediate 12G and 4-(1H-imidazol-2-yl)phenol were subjected to conditions as described for compound 2 to yield compound 4. Compound 4 as a white solid was obtained: ¹H NMR (Methanol-d₄, 400 MHz): δ 7.97-7.88 (m, 2H), 7.63-7.56 (m, 2H), 7.38 (s, 1H), 7.34-7.25 (m, 2H), 7.18 (s, 1H), 4.80-4.73 (m, 2H), 3.85 (d, J=2.3 Hz, 2H). MS (ESI) m/z (M+H)⁺ 248.1.

### (E)-2-((benzo[d]thiazol-4-yloxy)methyl)-3-fluoroprop-2-en-1-amine hydrochloride (8)

Intermediate 12F and benzo[d]thiazol-4-ol were subjected to conditions as described for compound 2 to yield compounds 8 and 9. The mixture was concentrated under vacuum. The product was purified by preparatory-HPLC (water (0.05% HCl)-ACN). Compound 8 (40 mg, yield: 87.9%) as a white solid was obtained: ¹H NMR (DMSO-d₆, 400 MHz): δ 9.32 (s, 1H), 8.42 (br s, 3H), 7.77 (d, J=8.0 Hz, 1H), 7.47-7.42 (m, 1H), 7.26-7.14 (m, 2H), 4.91 (br d, J=3.0 Hz, 2H), 3.67 (br d, J=4.3 Hz, 2H). MS (ESI) m/z (M+H)⁺238.9.

### (Z)-2-((benzo[d]thiazol-4-yloxy)methyl)-3-fluoroprop-2-en-1-amine hydrochloride (9)

Compound 9 (3 mg, yield: 6.6%) as a white solid was obtained: ¹H NMR (DMSO-d₆, 400 MHz): δ 9.32 (s, 1H), 7.76 (d, J=8.3 Hz, 1H), 7.52 (t, J=8.0 Hz, 1H), 7.30-7.05 (m, 2H), 5.09 (d, J=2.5 Hz, 2H), 3.83 (br s, 2H). MS (ESI) m/z (M+H)⁺ 238.9.

### (E)-2-((4-(1H-benzo[d]imidazol-2-yl)phenoxy)methyl)-3-fluoroprop-2-en-1-amine hydrochloride (10)

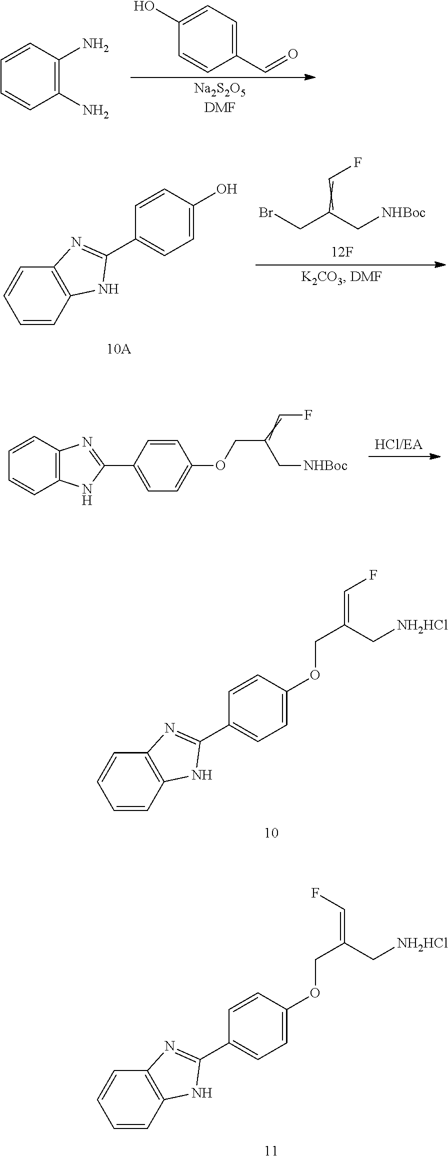

To a solution of benzene-1,2-diamine (200 mg, 1.85 mmol) and 4-hydroxybenzaldehyde (271 mg, 2.22 mmol) in DMF (5 mL) was added Na₂S₂O₅ (524 mg, 2.76 mmol), the mixture was stirred at 150° C. for 45 min under microwave. The mixture was added H₂O (50 mL) and extracted with EA (30 mL), the organic phase was washed with saturated NH₄Cl (30 mL), brine (30 mL) and dried over Na₂SO₄, filtered and concentrated under vacuum. The product was slurred with EA (10 mL), filtered to give the product. Compound 10A (366 mg, yield 94.13%) was obtained as a yellow solid. MS (ESI) m/z (M+H)⁺ 211.1.

Intermediates 10A and 12F were subjected to conditions as described for compound 2 to yield compound 10B. The mixture was concentrated under vacuum. The product was purified by preparatory-HPLC (water (0.05% HC)-ACN). Compound 10 (10 mg, yield: 16.07%) as a white solid was obtained: ¹H NMR (DMSO-d₆, 400 MHz): δ 8.34 (br d, J=8.3 Hz, 2H), 8.26 (br s, 3H), 7.82-7.77 (m, 2H), 7.49 (br s, 3H), 7.37-7.27 (m, 3H), 4.80 (br d, J=2.5 Hz, 2H), 3.64 (br d, J=5.0 Hz, 2H). (M+H)⁺ 298.1.

### (Z)-2-((4-(1H-benzo[d]imidazol-2-yl)phenoxy)methyl)-3-fluoroprop-2-en-1-amine hydrochloride (11)

Compound 11 (5 mg, yield: 8.04%) as a white solid was obtained: ¹H NMR (DMSO-d₆, 400 MHz): δ 8.34 (d, J=8.8 Hz, 2H), 8.24 (br s, 3H), 7.79 (dd, J=3.3, 6.0 Hz, 2H), 7.55-7.47 (m, 2H), 7.39-7.31 (m, 3H), 7.21-7.05 (m, 1H), 4.90 (s, 2H), 3.57 (br s, 2H). (M+H)⁺ 298.1.

### (E)-N-(4-((2-(aminomethyl)-3-fluoroalkyl)oxy)phenyl)propionamide (13)

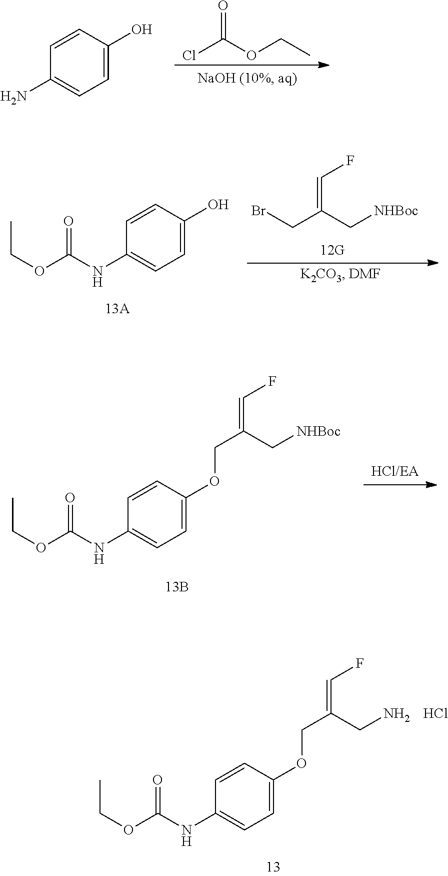

4-Aminophenol (0.9 mL, 9.16 mmol) was added to the mixture of ethyl carbonochloridate (1 g, 9.16 mmol) in NaOH (w/w %=10%, 10 mL) at 0° C. The mixture was stirred at 80° C. for 0.5 h. The mixture was cooled to 20° C. and adjusted to pH 1-2 by 6 N HCl, extracted with EA (150 mL). The organic phase was dried over Na₂SO₄, filtered and concentrated under vacuum. The product was purified by FCC (0-30% EA/PE) to afford compound 13A (900 mg, yield 54.21%) as a yellow solid.

Intermediates 13A and 12G were subjected to conditions as described for compound 2 to yield compound 13. Compound 13 (146 mg, yield: 67.82%) as a yellow solid was obtained: ¹H NMR (CD₃OD, 400 MHz): δ 7.33 (d, J=8.8 Hz, 2H), 7.17 (d, J=81.2 Hz, 1H), 6.95-6.89 (m, 2H), 4.58-4.54 (m, 2H), 4.14 (q, J=7.1 Hz, 2H), 3.79 (d, J=2.0 Hz, 2H), 1.28 (t, J=7.1 Hz, 3H). ¹⁹F NMR (CD₃OD, 376 MHz): −123.95, −124.17. MS (ESI) m/z (M+H)⁺ 269.1.

### (E)-N-(4-((2-(aminomethyl)-3-fluoroalkyl)oxy)phenyl)propionamide (82)

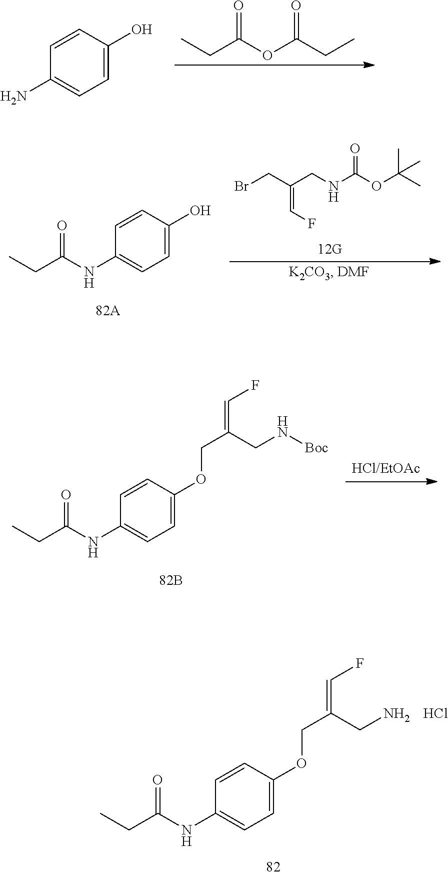

To a solution of 4-aminophenol (1 g, 9.16 mmol, 1.43 mL) and propanoyl propanoate (1.19 g, 9.16 mmol, 1.18 mL) in H₂O (10 mL) was added sodium dodecyl sulfate (40 mg, 138.71 umol). The mixture was stirred at 15° C. for 30 min. The reaction was diluted with EA (30 mL) and H₂O (30 mL), and extracted with EA (20 mL×3), combined the organic phases and washed with brine (30 mL), dried over Na₂SO₄, filtered, and concentrated. The residue was purified by flash silica gel chromatography (ISCO©; 24 g SepaFlash© Silica Flash Column, Eluent of 80% Ethyl acetate/Petroleum ether gradient @ 35 mL/min). Compound 82A (0.9 g, yield: 59.5%) was obtained as a yellow solid. ¹H NMR (400 MHz, DMSO-d₆) δ 9.55 (s, 1H), 9.11 (s, 1H), 7.33 (d, J=8.8 Hz, 2H), 6.75-6.59 (m, 2H), 2.23 (q, J=7.6 Hz, 2H), 1.04 (t, J=7.5 Hz, 3H). MS (ESI) m/z (M+H)⁺ 166.1.

Intermediates 82A and 12G were subjected to conditions as described for compound 2 to yield compound 82. Compound 82 (112 mg, yield: 54.46%) as a yellow oil was obtained: ¹H NMR (DMSO-d₆, 400 MHz): δ 9.87 (s, 1H), 8.35 (br s, 3H), 7.53 (d, J=8.8 Hz, 2H), 7.40-7.16 (m, 1H), 6.93 (d, J=9.0 Hz, 2H), 4.59 (d, J=3.3 Hz, 2H), 3.57 (br d, J=4.8 Hz, 2H), 2.34-2.25 (m, 2H), 1.06 (t, J=7.5 Hz, 3H). ¹⁹F NMR (CD₃OD, 376 MHz): −123.56. MS (ESI) m/z (M+H)⁺ 253.2.

### Example 6

### Compounds 14, 16-17, 19-20

### (E)-2-((benzo[d][1,3]dioxol-4-yloxy)methyl)-3-fluoroprop-2-en-1-amine hydrochloride (14)

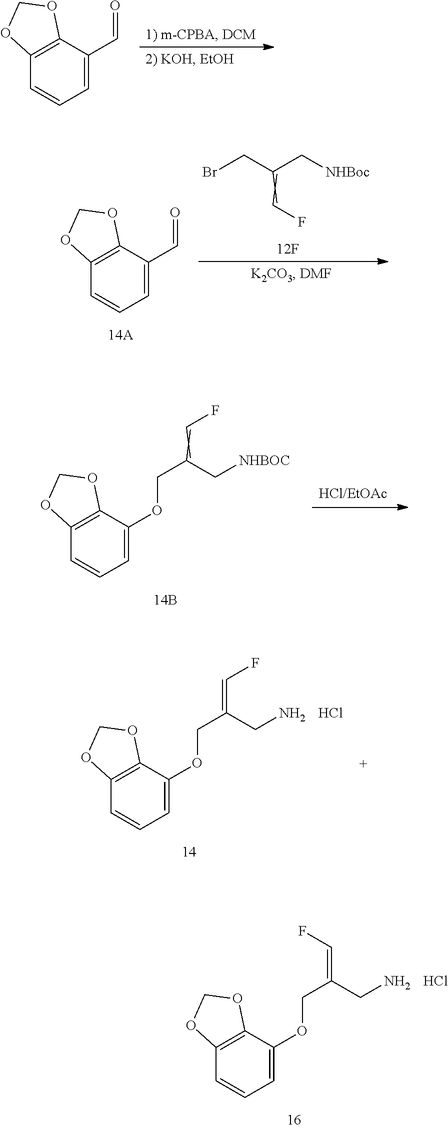

To a solution of benzo[d][1,3]dioxole-4-carbaldehyde (800 mg, 5.33 mmol) in DCM (20 mL) was added m-CPBA (1.38 g, 6.39 mmol, 80% purity). the mixture was stirred at 45° C. for 16 h. The reaction was filtered and the filtrate was washed with Na₂CO₃ (aqueous) (30 mL), and brine (30 mL), dried over Na₂SO₄, filtered and concentrated. The residue was treated with KOH (900 mg, 16.04 mmol) in EtOH (10 mL), and stirred at 20° C. for 16 h. The reaction was added H₂O (20 mL), extracted with EA (30 mL), the water phase was adjust with 1N HCl to pH 2-3, and extracted with EA (20 mL×2), dried over Na₂SO₄, filtered and concentrated. The residue was used to the next step without purification. Compound 14A (400 mg, yield: 54.4%) was obtained as a white solid. ¹H NMR (400 MHz, DMSO-d₆) δ 9.62 (s, 1H), 6.68-6.59 (m, 1H), 6.42-6.39 (m, 2H), 5.94-5.91 (s, 2H). MS (ESI) m/z (M+H)⁺ 139.1.

Intermediates 14A and 12F were subjected to conditions as described for compound 2 to yield compound 14B. The mixture was concentrated under vacuum. The product was purified by preparatory-HPLC (water (0.05% HCl)-ACN). Compound 14 (26 mg, yield: 52.94%) as a white solid was obtained: ¹H NMR (CD₃OD, 400 MHz): δ 7.15 (d, J=81.2 Hz, 1H), 6.81-6.75 (m, 1H), 6.62 (d, J=8.3 Hz, 1H), 6.56 (d, J=7.8 Hz, 1H), 5.93 (s, 2H), 4.70 (d, J=3.7 Hz, 2H), 3.80 (d, J=2.0 Hz, 2H). ¹⁹F NMR (CD₃OD, 376 MHz): δ −123.15, −123.37. MS (ESI) m/z (M+H)⁺ 226.1.

### (Z)-2-((benzo[d][1,3]dioxol-4-yloxy)methyl)-3-fluoroprop-2-en-1-amine hydrochloride (16)

Compound 16 (4 mg, yield: 8.09%) as a white solid was obtained: ¹H NMR (CD₃OD, 400 MHz): δ 7.08 (d, J=80.8 Hz, 1H), 6.85-6.79 (m, 1H), 6.68 (d, J=8.8 Hz, 1H), 6.60 (d, J=8.0 Hz, 1H), 5.97 (s, 2H), 4.94 (d, J=2.4 Hz, 2H), 3.72 (d, J=2.8 Hz, 2H). ¹⁹F NMR (CD₃OD, 376 MHz): δ−121.76. MS (ESI) m/z (M+H)⁺ 226.1.

### (E)-N-(2-(aminomethyl)-3-fluoroalkyl)benzenesulfonamide hydrochloride (17)

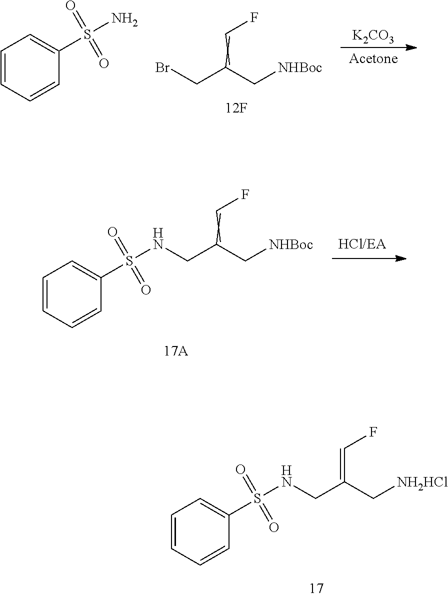

Benzenesulfonamide (29 mg, 186.48 umol) and intermediate 12F (50 mg, 186.48 umol) were subjected to treatment with K₂CO₃ (31 mg, 223.78 umol) in acetone (10 mL). The mixture was stirred at 60° C. for 3 h. The mixture was added H₂O (30 mL) and extracted with EA (20 mL×2). The organic phase was washed with brine (30 mL), dried over Na₂SO₄, filtered and concentrated under vacuum. Compound 17A (64 mg, purity 48.5%) was obtained as a yellow oil, which was used for next step without purification and then subjected to conditions as described for compound 2 to yield compound 17. The product was purified by preparatory-HPLC (water (0.05% HCl)-ACN). Compound 17 (15 mg, yield: 28.12%; HCl) as a yellow solid was obtained: ¹H NMR (CD₃OD, 400 MHz): δ 7.91 (d, J=7.3 Hz, 2H), 7.72-7.66 (m, 1H), 7.65-7.59 (m, 2H), 6.93 (d, J=81.6 Hz, 1H), 3.83-3.62 (m, 2H), 3.55 (d, J=2.0 Hz, 2H). ¹⁹F NMR (CD₃OD, 376 MHz): δ−125.16. MS (ESI) m/z (M+H)⁺ 245.1.

### (E)-3-fluoro-2-((4-(thiazol-2-yl)phenoxy)methyl)prop-2-en-1-amine hydrochloride (19)

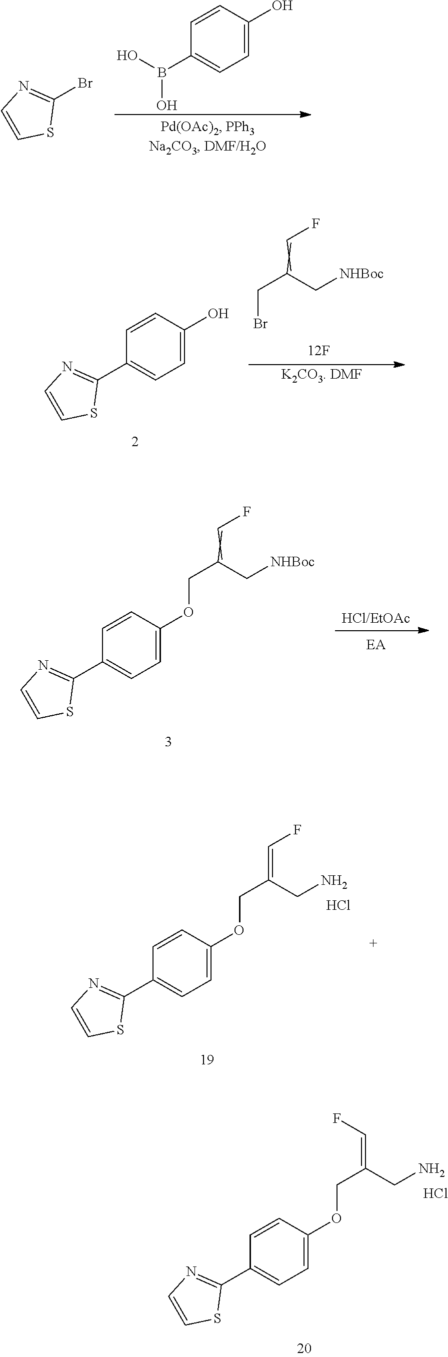

To a solution of (4-hydroxyphenyl)boronic acid (1.18 g, 8.54 mmol), Na₂CO₃ (1.94 g, 18.30 mmol) and PPh₃ (234 mg, 915.00 umol) in DMF (20 mL) was added to a solution of 2-bromothiazole (1 g, 6.10 mmol) in H₂O (7 mL) followed with Pd(OAc)₂ (41 mg, 183.00 umol). The mixture was stirred at 100° C. for 21 h under N2. The reaction mixture was diluted with water (40 mL) and extracted with ethyl acetate (30 mL×3). The combined organic phase was washed with water (20 mL×2) and brine (20 mL), dried over anhydrous Na₂SO₄, filtered and concentrated under reduced pressure. The crude product was purified by silica gel column (petroleum:ethyl acetate=(1:0 to 2:1) to afford compound 19A (343.9 mg, yield 29.27%) as brown solid. ¹H NMR (DMSO-d₆, 400 MHz) δ 9.96 (s, 1H), 7.84-7.70 (m, 3H), 7.60 (d, J=3.2 Hz, 1H), 6.83 (d, J=8.4 Hz, 2H). MS (ESI) m/z (M+H)⁺ 178.1.

Intermediates 19A and 12F were subjected to conditions as described for compound 2 to yield compounds 19 and 20. The mixture was concentrated under vacuum. The product was purified by preparatory-HPLC (water (0.05% HC)-ACN). Compound 19 (68.1 mg, yield: 63.21%) as a yellow solid was obtained: ¹H NMR (DMSO-d₆, 400 MHz) δ 8.38 (br. s., 3H), 7.92 (d, J=8.8 Hz, 2H), 7.87 (d, J=3.2 Hz, 1H), 7.72 (d, J=3.2 Hz, 1H), 7.34 (d, J=82.0 Hz, 1H), 7.13 (d, J=9.2 Hz, 2H), 4.72 (d, J=3.2 Hz, 2H), 3.60 (br. d., J=4.6 Hz, 2H). ¹⁹F NMR (DMSO-d₆, 376 MHz): −122.73. MS (ESI) m/z (M+H)⁺ 264.9.

### (Z)-3-Fluoro-2-((4-(Thiazol-2-Yl)Phenoxy)Methyl)Prop-2-En-1-Amine Hydrochloride (20)

Compound 20 (20.6 mg, yield: 18.09%) as a yellow solid was obtained: ¹H NMR (DMSO-d₆, 400 MHz) δ 8.32 (br. s., 3H), 7.90 (d, J=8.4 Hz, 2H), 7.85 (d, J=3.6 Hz, 1H), 7.70 (d, J=3.2 Hz, 1H), 7.34-7.11 (m, 3H), 4.80 (d, J=2.0 Hz, 2H), 3.51 (br s, 2H). ¹⁹F NMR (DMSO-d₆, 376 MHz): −122.35. MS (ESI) m/z (M+H)⁺ 264.9.

### Example 7

### Compounds 27-28, 33-34, 37-38, 40

### (E)-3-fluoro-2-((4-(1-methyl-1H-imidazol-2-yl)phenoxy)methyl)prop-2-en-1-aminedihydrochloride (27)

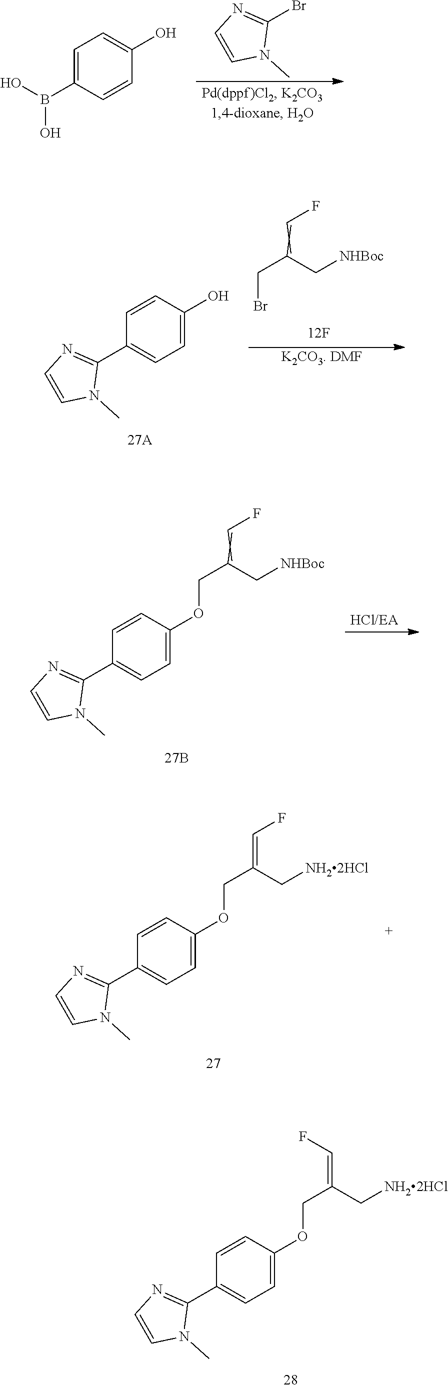

(4-hydroxyphenyl)boronic acid and 2-bromo-1-methyl-1H-imidazole were subjected to conditions as for compounds 19 and 20 to yield compounds 27 and 28. Compound 27 (80 mg, yield: 48.0%) as a white solid was obtained: ¹H NMR (400 MHz, DMSO-d₆) δ 14.97 (br s, 1H), 8.46 (br s, 3H), 7.82-7.68 (m, 4H), 7.43 (s, 1H), 7.28-7.18 (m, 2H), 4.82-4.74 (m, 2H), 3.82 (s, 3H), 3.57 (br s, 2H). MS (ESI) m/z (M+H)⁺ 262.2.

### (Z)-3-fluoro-2-((4-(thiazol-2-yl)phenoxy)methyl)prop-2-en-1-amine hydrochloride (28)

Compound 28 (10 mg, yield: 6%) as a white solid was obtained: ¹H NMR (400 MHz, DMSO-d₆) δ 14.94 (br s, 1H), 8.43 (br s, 3H), 7.85-7.66 (m, 4H), 7.37-7.12 (m, 3H), 4.86 (s, 2H), 3.82 (s, 3H), 3.59-3.46 (m, 2H). MS (ESI) m/z (M+H)⁺ 262.2.

### (E)-3-fluoro-2-((4-(pyrimidin-2-yl)phenoxy)methyl)prop-2-en-1-amine hydrochloride (37)

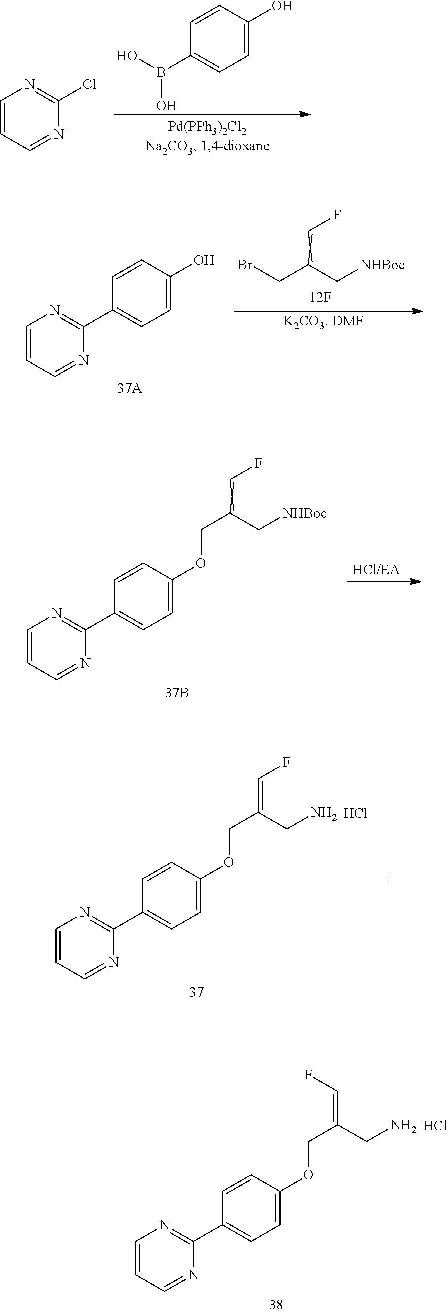

(4-hydroxyphenyl)boronic acid and 2-chloropyrimidine were subjected to conditions as for compounds 19 and 20 to yield compounds 37 and 38. Compound 37 (120 mg, yield: 57.0%) as a yellow solid was obtained: ¹H NMR (400 MHz, DMSO-d₆) δ 8.85 (d, J=4.8 Hz, 2H), 8.48-8.30 (m, 5H), 7.38 (t, J=4.8 Hz, 1H), 7.36 (d, J=82.4 Hz, 1H), 7.14 (d, J=8.8 Hz, 2H), 4.74 (d, J=3.2 Hz, 2H), 3.61 (d, J=4.4 Hz, 2H). ¹⁹F NMR (376 MHz, DMSO-d₆) δ−122.82. MS (ESI) m/z (M+H)⁺ 260.1.

### (Z)-3-fluoro-2-((4-(pyrimidin-2-yl)phenoxy)methyl)prop-2-en-1-amine hydrochloride (38)

Compound 38 (10 mg, yield: 4.5%) as a yellow solid was obtained: ¹H NMR (400 MHz, DMSO-d₆) δ 8.85 (d, J=4.8 Hz, 2H), 8.43-8.31 (m, 5H), 7.38 (t, J=5.2 Hz, 1H), 7.36 (s, 1.5H), 7.16-7.13 (m, 2.5H), 4.84 (d, J=2.0 Hz, 2H), 3.54 (d, J=2.4 Hz, 2H). ¹⁹F NMR (376M Hz, DMSO-d₆) δ−122.45. MS (ESI) m/z (M+H)⁺ 260.1.

### (E)-2-((4-(1H-imidazol-5-yl)phenoxy)methyl)-3-fluoroprop-2-en-1-amine (40)

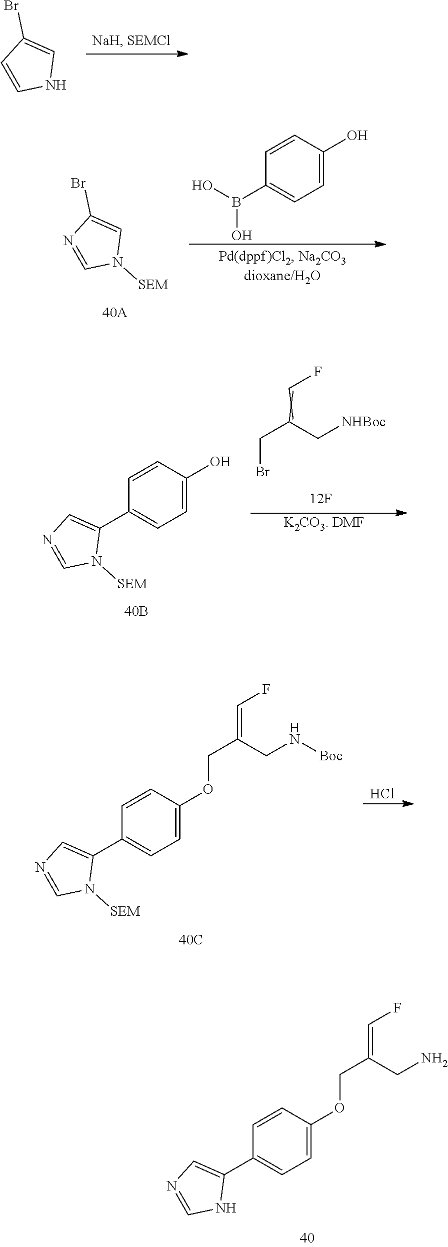

To a solution of 4-bromo-1H-imidazole (2 g, 13.61 mmol) in THF (30 mL) at 0° C. was added NaH (653 mg, 16.33 mmol, 60% purity) portionwise. The mixture was stirred at 0° C. for 2 h. Then SEM-Cl (2.72 g, 16.33 mmol, 2.9 mL) was added dropwise. The mixture was stirred at 20° C. for 12 h. The mixture was quenched with H₂O (30 mL), extracted with EtOAc (30 mL×2). The organics were collected, dried with Na₂SO₄, filtered and concentrated to give compound 40A (4 g, crude) as yellow oil, which was used directly for the next step without further purification.

(4-hydroxyphenyl)boronic acid and intermediate 40A-were subjected to conditions as for compounds 19 and 20 to yield compound 40. Compound 40 (20 mg, yield: 27.5%) as a white solid was obtained: MS (ESI) m/z (M+H)⁺ 248.1. ¹H NMR (400 MHz, D2O) δ 7.71 (s, 1H), 7.64-7.56 (m, 2H), 7.34 (s, 1H), 7.04-6.93 (m, 2.8H), 6.80-6.71 (m, 0.5H), 4.57-4.49 (m, 2H), 3.51-3.43 (m, 2H). (M+H)⁺ 248.1

### Compounds 33-34

### (E)-2-(([1,1′-biphenyl]-4-yloxy)methyl)-3-fluoroprop-2-en-1-amine hydrochloride (33)

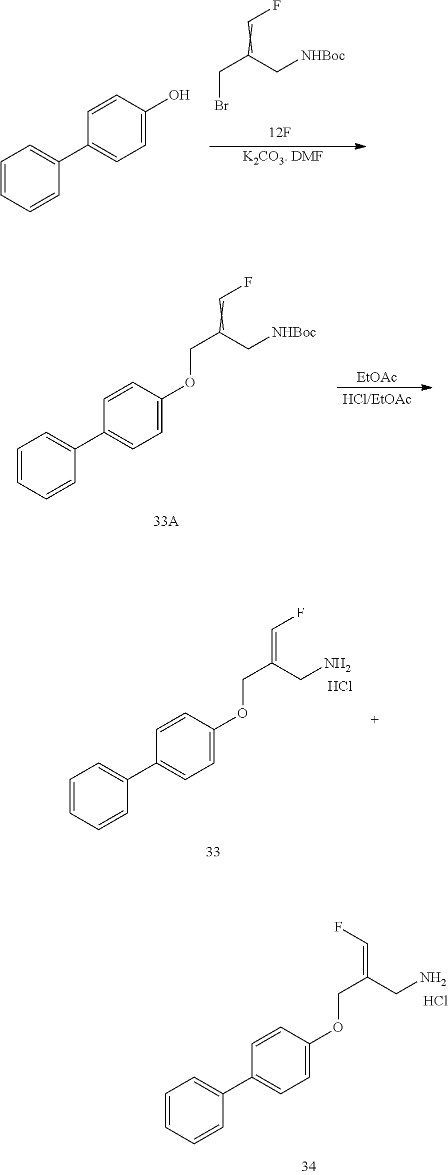

[1,1′-Biphenyl]-4-ol and 12F were subjected to conditions as described for compound 2 to yield compound 33A. The mixture was concentrated under vacuum. The product was purified by preparatory-HPLC (water (0.05% HCl)-ACN). Compound 33 (55 mg, yield: 37.0%) as a white solid was obtained: ¹H NMR (DMSO-d₆, 400 MHz): δ 8.35 (brs, 3H), 7.68-7.57 (m, 4H), 7.48-7.21 (m, 4H), 7.22 (d, J=10.0 Hz, 2H), 4.69 (d, J=3.2 Hz, 2H), 3.61 (s, 2H). ¹⁹F NMR (376 MHz, DMSO-d₆): δ−123.20). MS (ESI) m/z (M+H)⁺ 258.0.

### (Z)-2-(([1,1′-biphenyl]-4-yloxy)methyl)-3-fluoroprop-2-en-1-amine hydrochloride (34)

Compound 34 (5 mg, yield: 3.1%) as a white solid was obtained: ¹H NMR (DMSO-d₆, 400 MHz): δ 8.16 (brs, 3H), 7.68-7.57 (m, 4H), 7.48-7.40 (m, 2H), 7.36-7.06 (m, 4H), 4.78 (d, J=2.0 Hz, 2H), 3.55 (s, 2H). ¹⁹F NMR (376 MHz, DMSO-d₆): δ−122.52). MS (ESI) m/z (M+H)⁺ 258.0.

### Example 8

### Compounds 43-44

### (E)-2-((4-(1,2,4-oxadiazol-3-yl)phenoxy)methyl)-3-fluoroprop-2-en-1-amine hydrochloride (43)

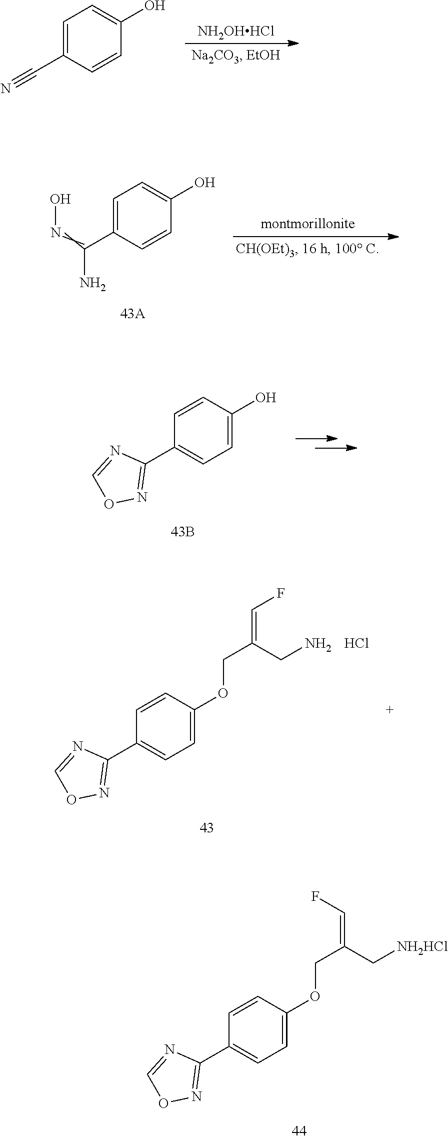

To a solution of 4-hydroxybenzonitrile (3.00 g, 25.2 mmol) and NH₂OH.HCl (2.63 g, 37.8 mmol) in EtOH (150 mL) was added Na₂CO₃ (5.34 g, 50.4 mmol), then it was stirred at 78° C. for 16 h. After cooling to room temperature, the reaction mixture was poured into water (200 mL), and then it was extracted with EtOAc (200 mL×6). The combined organic layer was dried over anhydrous Na₂SO₄ and then concentrated to afford compound 43A (2.9 g, crude) as an off-white solid. 2.1 g of this crude product was purified by column chromatography (SiO₂, EtOAc:MeOH=10:1) to afford compound 43A (1.2 g, yield: 31.3%) as an off-white solid. The water phase was concentrated under reduced pressure to remove a part of water and then it was extracted with EtOAc (150 mL×4). The combined organic layer was dried over anhydrous Na₂SO₄, filtered and then concentrated to afford compound 43A (650 mg, yield: 17.0%) as an off-white solid. ¹H NMR (CDCl₃, 400 MHz): δ 9.60 (S, 1H), 9.36 (s, 1H), 7.48 (d, J=8.8 Hz 2H), 6.73 (d, J=8.8 Hz, 2H), 5.64 (s, 2H).

To a mixture of compound 43A (1.45 g, 9.53 mmol) in triethoxymethane (10 mL) was added montmorillonite (1.08 g, 3.81 mmol). The mixture was stirred at 100° C. for 16 h. The mixture was concentrated under reduced pressure to give the residue. The residue was purified by column chromatography (SiO₂, 20% PE in EtOAc) to afford compound 43B (160 mg, yield: 10.4%) as brown solid. ¹H NMR (CDCl₃, 400 MHz): δ 10.15 (brs, 1H), 9.59 (s, 1H), 7.88-7.81 (m, 2H), 6.94-6.87 (m, 2H).

Intermediate 43B and 12F were subjected to conditions as described for compound 2 to yield compound 33A. The mixture was concentrated under vacuum. The product was purified by preparatory-HPLC (water (0.05% HCl)-ACN). Compound 43 (30.6 mg, yield: 13.4%) as an off-white solid was obtained: ¹H NMR (DMSO-d₆, 400 MHz): δ 9.66 (s, 1H), 8.22 (br.s., 3H), 8.01 (d, J=8.8 Hz, 2H), 7.36 (d, J=82.0 Hz, 1H), 7.19 (d, J=8.8 Hz, 2H), 4.72 (d, J=2.8 Hz, 2H), 3.63 (br.s., 2H). ¹⁹F NMR (376 MHz, DMSO-d₆): δ=−122.52. MS (ESI) m/z (M+H)⁺ 250.0.

### (Z)-2-((4-(1,2,4-OXADIAZOL-3-YL)PHENOXY)METHYL)-3-FLUOROPROP-2-EN-1-AMINE HYDROCHLORIDE (44)

Compound 44 (6.8 mg, yield: 2.8%) as an off-white solid was obtained: ¹H NMR (DMSO-d₆, 400 MHz): δ 9.65 (s, 1H), 8.30 (br.s., 3H), 8.00 (d, J=8.4 Hz, 2H), 7.24 (d, J=82.4 Hz, 1H), 7.20 (d, J=8.8 Hz, 2H), 4.83 (s, 2H), 3.55 (br.s., 2H). ¹⁹F NMR (376 MHz, DMSO-d₆): δ-122.06. MS (ESI) m/z (M+H)⁺ 250.0.

### Example 9

### Compounds 22,26, 45-46, and 62

### (E)-N1-cyclohexyl-2-(fluoromethylene)propane-1,3-diamine (22)

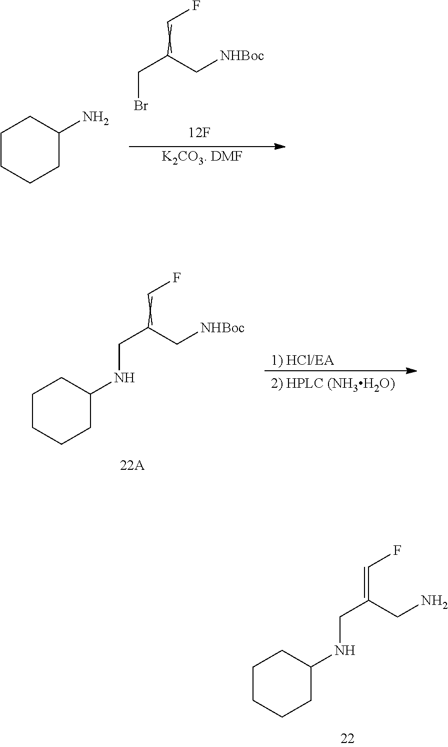

Cyclohexylamine and intermediate 12F were subjected to conditions as described for compound 2 to yield the intermediate 22A and this was then treated with HCl as for compound 2 to yield compound 22. The product was purified by preparatory-HPLC [water (0.04% NH₃H₂O+10 mM NH₄HCO₃)-ACN] to afforded compound 22 (6.1 mg, yield: 8.53%) as a yellow solid. ¹H NMR (DMSO-d₆, 400 MHz): δ 6.63 (d, J=87.2 Hz, 1H), 3.21 (s, 2H), 3.10 (d, J=2.4 Hz, 2H), 2.36-2.21 (m, 1H), 1.86-1.70 (m, 2H), 1.69-1.57 (m, 2H), 1.56-1.46 (m, 1H), 1.27-1.05 (m, 3H), 1.04-2.87 (m, 2H). ¹⁹F NMR (376 MHz, DMSO-d₆): δ=−137.08.

### (E)-N¹-benzyl-2-(fluoromethylene)propane-1,3-diamine (26)

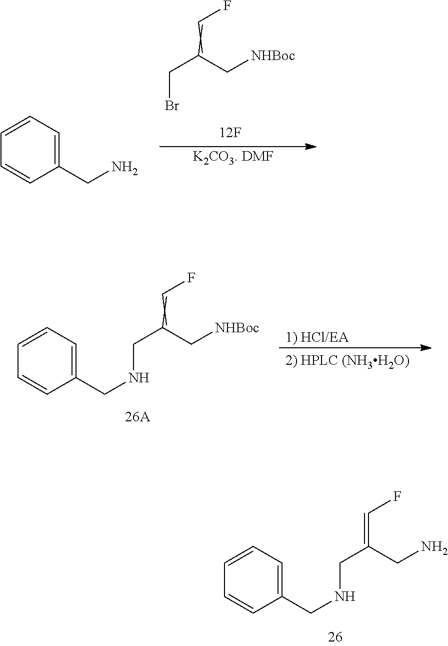

Benzylamine and intermediate 12F were subjected to conditions as described for compound 2 to yield the intermediate 26A and this was then treated with HCl as for compound 2 to yield compound 26. The product was purified by preparatory-HPLC [water (0.04% NH₃H₂O+10 mM NH₄HCO₃)-ACN] to afforded compound 26 (25.0 mg, yield: 37.6%) as a white solid. ¹H NMR (400 MHz, DMSO-d₆) δ 7.36-7.27 (m, 4H), 7.26-7.18 (m, 1H), 6.79 (d, J=85.6 Hz, 1H), 3.62 (s, 2H), 3.42-3.36 (m, 2H), 3.14 (s, 2H). ¹⁹F NMR (376 MHz, DMSO-d₆) δ−131.84. MS (ESI) m/z (M+H)⁺ 195.2.

### (E)-4-((2-(aminomethyl)-3-fluoroalkyl)amino)-n-(tert-butyl)benzamide hydrochloride (45)

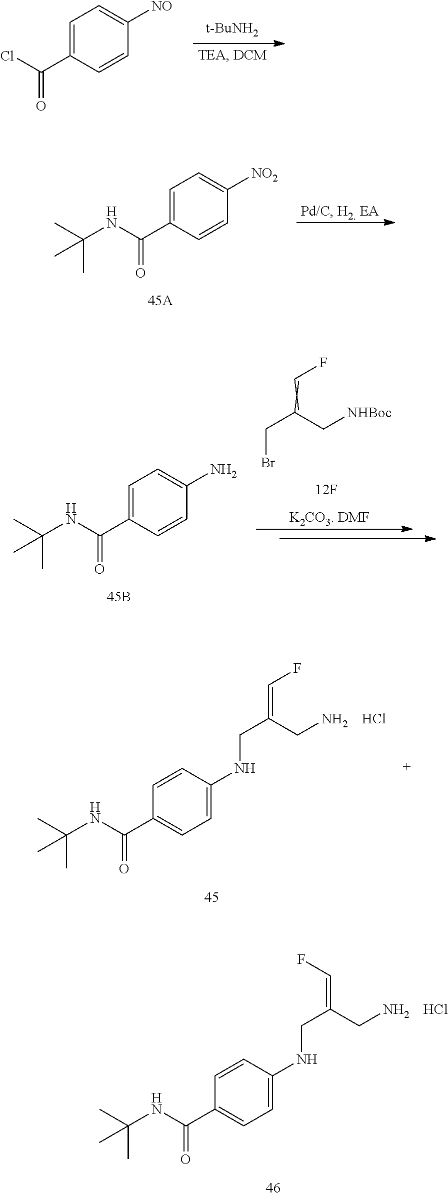

To a mixture of 2-methylpropan-2-amine (2.17 g, 29.6 mmol) and TEA (8.18 g, 80.8 mmol) in DCM (20 mL) was added 4-nitrobenzoyl chloride (5.00 g, 26.9 mmol) at 0° C. (ice/water). The resultant mixture was stirred for 12 h with gradual warming to 20° C. The reaction mixture was diluted with 0.5N HCl (30 mL) and extracted with DCM (40 mL×2). The combined organic layers were washed with NaHCO₃ (30 mL) and brine (30 mL), dried over Na₂SO₄, filtered and concentrated under reduced pressure to give a crude product 45A (4.2 g, crude) as a white solid, which was used in the next step without purification. ¹H NMR (400 MHz, DMSO-d₆) δ 8.30-8.24 (m, 2H), 8.14 (s, 1H), 8.04-7.98 (m, 2H), 1.39 (s, 9H).

To a solution of compound 45A (4.20 g, 18.9 mmol) in EA (40 mL) was added Pd/C (400 mg, 1.89 mmol) under N2. The suspension was degassed under vacuum and purged with H₂ several times. The mixture was stirred under H₂ (50 psi) at 20° C. for 12 hours. The mixture was filtered and concentrated under reduced pressure to give compound 45B (3.8 g, crude) as yellow oil, which was used in the next step without purification. ¹H NMR (400 MHz, DMSO-d₆) δ 7.53 (d, J=8.8 Hz, 2H), 7.22 (s, 1H), 6.50 (d, J=8.4 Hz, 2H), 5.53 (s, 2H), 1.34 (s, 9H).

Intermediates 45B and 12F were subjected to conditions as described for compound 2 to yield the intermediate which was then treated with HCl as for compound 2 to yield compounds 45 and 46. The product was purified by preparatory-HPLC [water (0.05% HCl)-ACN] to afforded compound 45 (40.0 mg, yield: 17.8%) as a white solid. ¹H NMR (400 MHz, DMSO-d₆) δ 8.42 (br. s., 3H), 7.61 (d, J=8.8 Hz, 2H), 7.32 (br. s., 1H), 7.11 (d, J=83.6 Hz, 1H), 6.65 (d, J=8.4 Hz, 2H), 3.85 (s, 2H), 3.47 (d, J=4.8 Hz, 2H), 1.34 (s, 9H). ¹⁹F NMR (376 MHz, DMSO-d₆) δ−127.01 (s, 1F). MS (ESI) m/z (M+H)⁺ 280.2.

### (Z)-4-((2-(aminomethyl)-3-fluoroalkyl)amino)-N-(tert-butyl)benzamide hydrochloride (46)

Compound 46 (10 mg, yield: 4.2%) as a yellow oil was obtained: ¹H NMR (400 MHz, DMSO-d₆) δ 8.34 (br. s, 3H), 7.61 (d, J=8.4 Hz, 2H), 7.30 (br. s., 1H), 7.15 (d, J=83.6 Hz, 1H), 6.55 (d, J=8.8 Hz, 2H), 3.95 (s, 2H), 3.31 (br. s., 2H), 1.34 (s, 9H). ¹⁹F NMR (376 MHz, DMSO-d₆) δ−126.05 (s, 1F). MS (ESI) m/z (M+H)⁺ 280.2.

### (E)-3-fluoro-2-(indolin-1-ylmethyl)prop-2-en-1-amine hydrochloride (62)

Indoline and intermediate 12F were subjected to conditions as described for compound 2 to yield the intermediate which was then treated with HCl as for compound 2 to yield compound 62. The product was purified by preparatory-HPLC [water (0.05% HCl)-ACN]to afforded compound 62 (30.0 mg, yield: 37.8%) as a yellow solid. ¹H NMR (400 MHz, D₂O) δ 7.47-7.32 (m, 4H), 7.19-6.94 (m, 1H), 4.19-4.12 (m, 2H), 3.89-3.78 (m, 2H), 3.73-3.62 (m, 2H), 3.27-3.17 (m, 2H). MS (ESI) m/z (M+H)⁺ 206.9.

### Example 10

### Compounds 25, 53, 39

### (E)-3-fluoro-2-((4-(oxazol-2-yl)phenoxy)methyl)prop-2-en-1-amine hydrochloride (25)

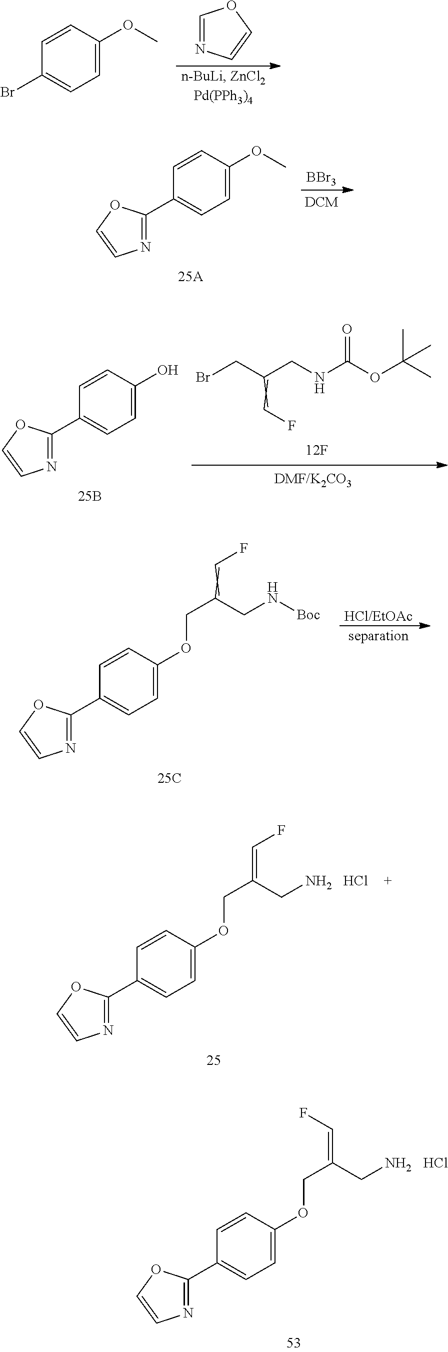

n-BuLi (2.5M, 7 mL) was added to the mixture of oxazole (665 mg, 9.62 mmol) in THF (55 mL) at −78° C. for 30 min. Then ZnCl₂ (6.70 g, 49.19 mmol) was added to the mixture at −78° C., then the mixture was stirred at 20° C. for 1.5 h. 1-Bromo-4-methoxybenzene (1 g, 5.35 mmol) and Pd(PPh₃)₄ (1.24 g, 1.07 mmol) was added to the mixture, then the mixture was stirred at 65° C. for 18 h. The mixture was added H₂O (100 mL) and extracted with EA (100 mL), filtered and the organic phase was washed with brine (30 mL), dried over Na₂SO₄, filtered and concentrated under vacuum. The product was purified by FCC (20% EA/PE). Compound 25A (90 mg, yield 9.61%) was obtained as a yellow oil. ¹H NMR (400 MHz, DMSO-d₆) δ 8.15 (s, 1H), 7.92 (d, J=8.8 Hz, 2H), 7.32 (s, 1H), 7.09 (d, J=8.8 Hz, 2H), 3.89-3.79 (m, 3H).

BBr₃ (1.03 mmol, 0.1 mL) was added to the mixture of compound 25A (60 mg, 342.50 umol) in DCM (10 mL). The mixture was stirred at −78° C. for 1 h. Then the mixture was stirred at 20° C. for 20 h and concentrated under vacuum. The mixture was added ice-H₂O (20 mL) and extracted with DCM (20 mL), the organic phase was dried over Na₂SO₄, filtered and concentrated under vacuum. Compound 25B (60 mg, yield 83.48%) was obtained as a yellow oil, which was used for next step without purification. MS (ESI) m/z (M+H)⁺ 162.1

Intermediates 25B and 12F were subjected to conditions as described for compound 2 to yield compounds 25 and 53. The mixture was concentrated under vacuum. The product was purified by preparatory-HPLC (water (0.05% HCl)-ACN). Compound 25 (12 mg, yield: 15.29%) as a yellow solid was obtained: ¹H NMR (CD₃OD, 400 MHz): δ 8.17 (d, J=1.0 Hz, 1H), 8.08 (d, J=9.0 Hz, 2H), 7.57 (d, J=0.8 Hz, 1H), 7.40-7.18 (m, 3H), 4.76 (d, J=3.5 Hz, 2H), 3.86 (s, 2H). ¹⁹F NMR (CD₃OD, 376 MHz): δ−122.56, −122.58. MS (ESI) m/z (M+H)⁺ 249.1.

### (Z)-3-fluoro-2-((4-(oxazol-2-yl)phenoxy)methyl)prop-2-en-1-amine hydrochloride (53)

Compound 53 (4 mg, yield: 5.09%) as a yellow solid was obtained: ¹H NMR (CD₃OD, 400 MHz): δ 8.10-8.02 (m, 3H), 7.45 (s, 1H), 7.27-7.04 (m, 3H), 4.93 (br. s., 2H), 3.74 (br. s., 2H). ¹⁹F NMR (CD₃OD, 376 MHz): δ−121.16. MS (ESI) m/z (M+H)⁺249.1.

### (E)-3-fluoro-2-((4-(1-methyl-1H-imidazol-4-yl)phenoxy)methyl)prop-2-en-1-amine (39)

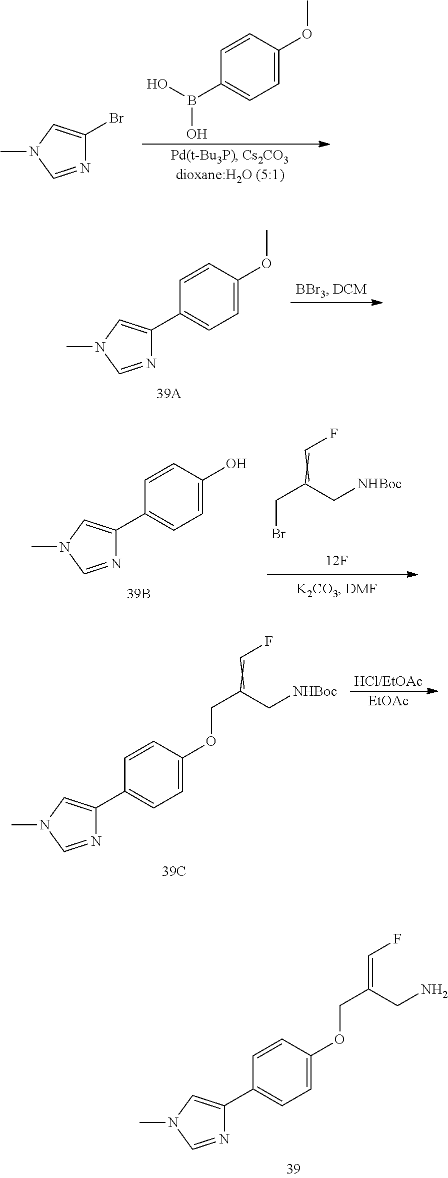

To a solution of 4-bromo-1-methyl-1H-imidazole (300 mg, 1.86 mmol), (4-methoxyphenyl)boronic acid (283 mg, 1.86 mmol) in dioxane (15 mL) was added to a solution of Cs₂CO₃ (1.2 g, 3.73 mmol) in H₂O (3 mL) followed with Pd(t-Bu₃P)₂ (95 mg, 186.34 umol). The mixture was stirred at 50° C. for 19 h under N2. The mixture was diluted with water (30 mL), extracted with ethyl acetate (20 mL×3). The combined organic phase was washed with brine (15 mL), dried over anhydrous Na₂SO₄, filtered and concentrated under reduced pressure. The residue was purified by silica gel column (petroleum:ethyl acetate=(1:0 to 2:3) to afford compound 39A (232.2 mg, yield 60.11%) as light red solid. ¹H NMR (CD₃OD, 400 MHz) δ 7.64-7.57 (m, 3H), 7.30 (s, 1H), 6.90 (br d, J=8.3 Hz, 2H), 3.79 (s, 3H), 3.73 (s, 3H). MS (ESI) m/z (M+H)⁺ 188.9.

Intermediate 39A was subjected to conditions as for compound 25 and it yielded compound 39. The product was purified by preparatory-HPLC [water (0.05% ammonia hydroxide v/v)-ACN]. Compound 39 (13.3 mg, yield: 33.84%) as a white solid was obtained: ¹H NMR (DMSO-d₆, 400 MHz) δ 7.63 (d, J=8.8 Hz, 2H), 7.57 (s, 1H), 7.45 (d, J=1.0 Hz, 1H), 6.96 (d, J=85.2 Hz, 1H), 6.97-6.92 (m, 2H), 4.55-4.53 (m, 2H), 3.65 (s, 3H), 3.31-3.30 (m, 2H). ¹⁹F NMR (376 MHz, DMSO-d₆) δ=−132.67. MS (ESI) m/z (M+H)⁺ 262.1.

### Example 11

### Compounds 48-50, 54

### (E)-3-fluoro-2-((4-(1-methyl-1H-pyrazol-5-yl)phenoxy)methyl)prop-2-en-1-amine hydrochloride (48)

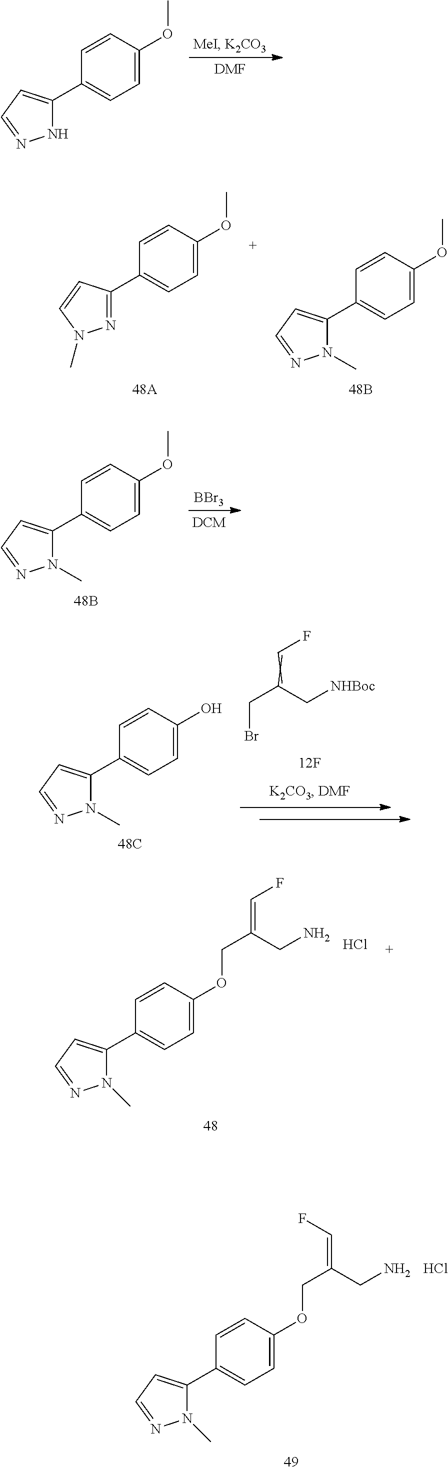

CH₃I (0.07 mL, 1.18 mmol) was added to the mixture of 5-(4-methoxyphenyl)-1H-pyrazole (100 mg, 574.06 umol) and K₂CO₃ (238 mg, 1.72 mmol) in DMF (5 mL). The mixture was stirred at 20° C. for 20 h. The mixture was added H₂O (50 mL) and extracted with EA (30 mL×2), the organic phase was washed with brine (30 mL), dried over Na₂SO₄, filtered and concentrated under vacuum. The product was purified by preparatory-SFC (0.1% NH₃H₂O/EtOH). Compound 48B (450 mg, yield 38.7%) was obtained as a yellow oil. ¹H NMR (400 MHz, DMSO-d₆) δ 7.58-7.30 (m, 3H), 7.05 (d, J=7.8 Hz, 2H), 6.32 (d, J=1.8 Hz, 1H), 3.82 (s, 3H), 3.81 (s, 3H). Compound 48A (232 mg, yield 19.9%) was obtained as a white solid. ¹H NMR (400 MHz, DMSO-d₆) δ 7.76-7.66 (m, 3H), 6.95 (d, J=7.9 Hz, 2H), 6.58 (d, J=2.3 Hz, 1H), 3.85 (s, 3H), 3.77 (s, 3H).

Intermediate 48B was subjected to conditions as for compound 25 and it yielded mixture of compound 48 and 49. The product was purified by preparatory-HPLC [water (0.05% HCl)-ACN]. Compound 48 (30 mg, yield: 20.79%) as a yellow solid was obtained: ¹H NMR (Methanol-d₄, 400 MHz): δ 8.13 (d, J=2.8 Hz, 1H), 7.61 (d, J=8.8 Hz, 2H), 7.42-7.12 (m, 3H), 6.76 (d, J=2.5 Hz, 1H), 4.75 (d, J=3.5 Hz, 2H), 4.06 (s, 3H), 3.87 (s, 2H).

### (Z)-3-fluoro-2-((4-(1-methyl-1H-pyrazol-5-yl)phenoxy)methyl)prop-2-en-1-amine hydrochloride (49)

Compound 49 (6 mg, yield: 4.16%) as a yellow solid was obtained: ¹H NMR (Methanol-d₄, 400 MHz): δ 8.01 (d, J=2.5 Hz, 1H), 7.61-7.55 (m, 2H), 7.27-7.03 (m, 3H), 6.68 (d, J=2.5 Hz, 1H), 4.93 (d, J=2.8 Hz, 2H), 4.02 (s, 3H), 3.75 (br s, 2H).

### (E)-3-fluoro-2-((4-(1-methyl-1H-pyrazol-3-yl)phenoxy)methyl)prop-2-en-1-amine hydrochloride (50)

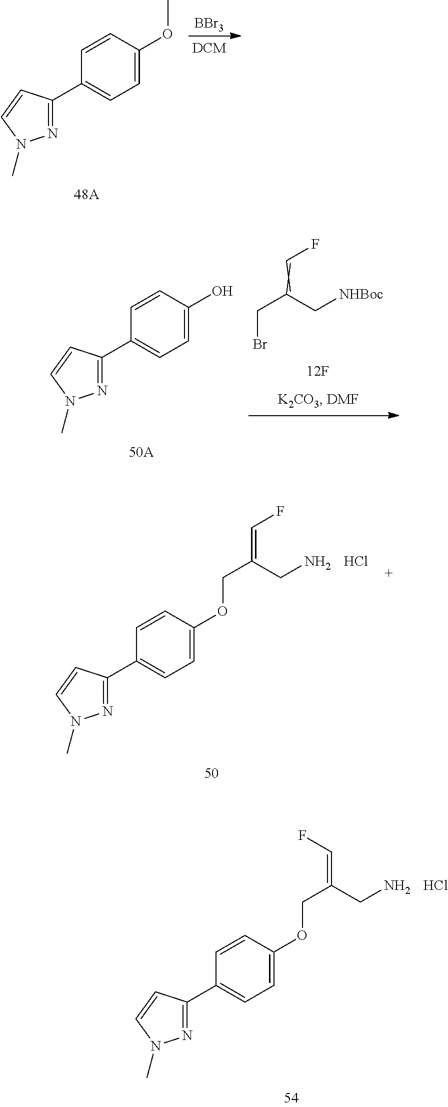

Intermediate 48A was subjected to conditions as for compound 25 and it yielded mixture of compound 50 and 54. The product was purified by preparatory-HPLC [water(0.05% HCl)-ACN]. Compound 50 (80 mg, yield: 32.3%) as a white solid was obtained: ¹H NMR (400 MHz, DMSO-d₆) δ 8.44 (br. s., 3H), 7.73 (s, 1H), 7.71 (s, 2H), 7.31 (d, J=82.4 Hz, 1H), 7.02 (d, J=8.8 Hz, 2H), 6.62 (d, J=2.0 Hz, 1H), 4.67 (d, J=2.8 Hz, 2H), 3.86 (s, 3H), 3.58 (d, J=4.8 Hz, 2H). ¹⁹F NMR (376 MHz, DMSO-d₆) δ−123.40 (s, 1F). MS (ESI) m/z (M+H)⁺ 262.2.

### (Z)-3-fluoro-2-((4-(1-methyl-1H-pyrazol-3-yl)phenoxy)methyl)prop-2-en-1-amine hydrochloride (54)

Compound 54 (10 mg, yield: 4.2%) as a white solid was obtained: ¹H NMR (400 MHz, DMSO-d₆) δ 8.35 (br. s., 3H), 7.73 (s, 1H), 7.72-7.69 (m, 2H), 7.23 (d, J=82.4 Hz, 1H), 7.02 (d, J=8.8 Hz, 2H), 6.61 (d, J=2.0 Hz, 1H), 4.77 (d, J=2.0 Hz, 2H), 3.91-3.80 (m, 3H), 3.52 (d, J=2.0 Hz, 2H). ¹⁹F NMR (376 MHz, DMSO-d₆) δ−122.81 (s, 1F). MS (ESI) m/z (M+H)⁺ 262.2.

### Example 12

### Compounds 51-52, 42

### (E)-2-((4-(1H-tetrazol-5-yl)phenoxy)methyl)-3-fluoroprop-2-en-1-amine hydrochloride (51)

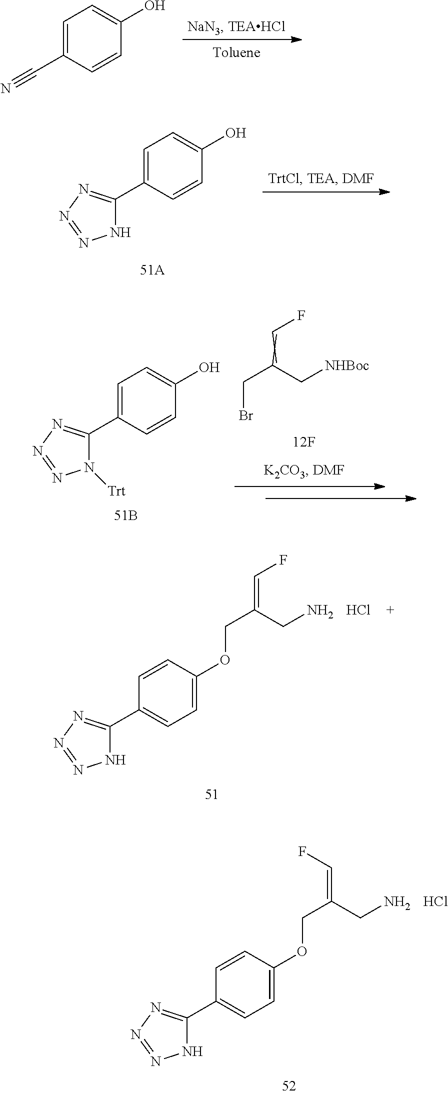

NaN₃ (4.91 g, 75.55 mmol) was added to the mixture of 4-hydroxybenzonitrile (3 g, 25.18 mmol) and N,N-diethylethanamine; hydrochloride (10.40 g, 75.55 mmol) in Toluene (80 mL). The mixture was stirred at 100° C. for 20 h. The mixture was cooled to room temperature and extracted with H₂O (200 mL). The aqueous phase was treated dropwise with 1N HCl to precipitate the product from the reaction mixture. The precipitate was collected by vacuum filtration and dried under vacuum to afford the product. Compound 51A (4 g, yield 97.95%) was obtained as a brown solid, which was used for next step without purification. ¹H NMR (400 MHz, DMSO-d₆) δ 10.20 (br s, 1H), 7.86 (br d, J=8.8 Hz, 2H), 6.96 (d, J=8.5 Hz, 2H).

TrtCl (378 mg, 1.36 mmol) was added to the mixture of compound 51A (200 mg, 1.23 mmol) and TEA (0.4 mL, 2.71 mmol) in DMF (10 mL) at 0° C., the mixture was stirred at 20° C. for 1.5 h. The mixture was quenched by H₂O (80 mL) and extracted with EA (50 mL×2), the organic phase was washed with brine (50 mL), dried over Na₂SO₄, filtered and concentrated under vacuum. Compound 51B (500 mg, yield 94.21%) was obtained as a white solid, which was used for next step without purification.

Intermediate 51B was subjected to conditions as for compound 25 and it yielded mixture of compound 51 and 52. The product was purified by preparatory-HPLC [water(0.05% HCl)-ACN]. Compound 51 (63 mg, yield: 39.34%) as a white solid was obtained: ¹H NMR (DMSO-d₆, 400 MHz): δ8.23 (br s, 3H), 8.05 (d, J=8.8 Hz, 2H), 7.48-7.26 (m, 1H), 7.48-7.26 (m, 1H), 7.23 (d, J=9.0 Hz, 2H), 4.73 (d, J=3.0 Hz, 2H), 3.63 (br s, 2H). MS (ESI) m/z (M+H)⁺ 250.1.

### (Z)-2-((4-(1H-tetrazol-5-yl)phenoxy)methyl)-3-fluoroprop-2-en-1-amine hydrochloride (52)

Compound 52 (9 mg, yield: 5.63%) as a white solid was obtained: ¹H NMR (Methanol-d₄, 400 MHz): δ 8.05-7.99 (m, 2H), 7.28-7.04 (m, 3H), 4.93 (d, J=2.5 Hz, 2H), 3.74 (br s, 2H). MS (ESI) m/z (M+H)⁺ 250.1.

### (E)-2-((4-(1H-pyrazol-5-yl)phenoxy)methyl)-3-fluoroprop-2-en-1-amine hydrochloride (42)

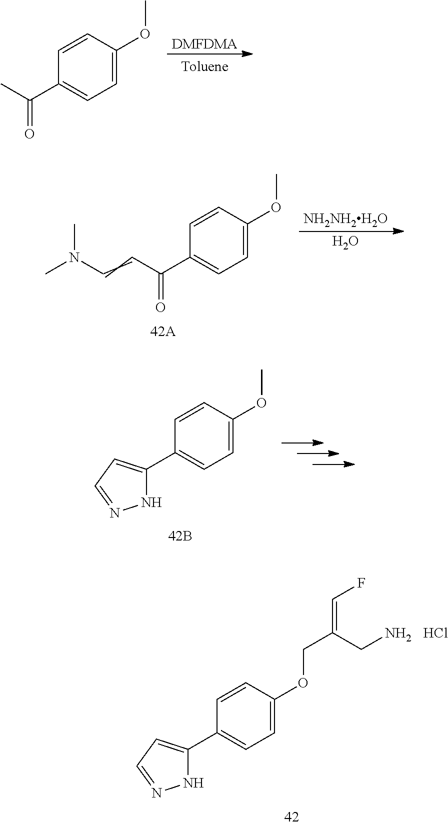

DMFDMA (12 mL, 91.83 mmol) was added to a solution of 1-(4-methoxyphenyl)ethan-1-one (9.8 g, 65.59 mmol) in toluene (66 mL). The mixture was stirred at 110° C. for 18 h. After cooled to the room temperature, the reaction mixture was diluted with water (40 mL) and extracted with ethyl acetate (30 mL×3). The organic phase was washed with brine (30 mL), dried over anhydrous Na₂SO₄, filtered and concentrated under reduced pressure. The residue was purified by silica gel column (petroleum ether; ethyl acetate=1:0 to 2:1) to afford compound 42A (1.8 g, yield 13.37%) as yellow solid. ¹H NMR (CDCl₃, 400 MHz): δ 7.92-7.87 (m, 2H), 7.78 (d, J=12.2 Hz, 1H), 6.92-6.88 (m, 2H), 5.70 (d, J=12.5 Hz, 1H), 3.84 (s, 3H), 3.20-2.81 (m, 6H).

Intermediate 42A was subjected to conditions as for compound 25 and it yielded compound 42. The product was purified by preparatory-HPLC [water(0.05% HCl)-ACN]. Compound 42 (39.7 mg, yield: 24.18%) as a yellow solid was obtained: ¹H NMR (400 MHz, DMSO-d₆) δ 8.38 (br. s., 3H), 7.85-7.77 (m, 3H), 7.32 (d, J=82.0 Hz, 1H), 7.07 (d, J=8.8 Hz, 2H), 6.75 (d, J=2.3 Hz, 1H), 4.69 (br. d., J=3.2 Hz, 2H), 3.61-3.59 (m, 2H). ¹⁹F NMR (376 MHz, DMSO-d₆) δ=−123.15. MS (ESI) m/z (M+H)⁺ 248.2.

### Example 13

### Compounds 29-30, 41

### (E)-3-fluoro-2-((4-(oxazol-4-yl)phenoxy)methyl)prop-2-en-1-amine hydrochloride (29)

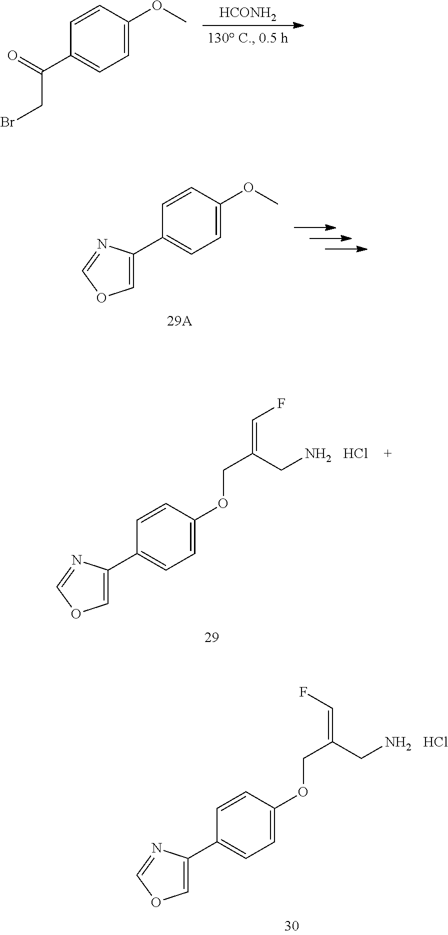

A solution of 2-bromo-1-(4-methoxyphenyl)ethan-1-one (2.0 g, 8.73 mmol) in HCONH₂ (8 mL) was stirred at 130° C. for 1 h under N2 atmosphere. After cooling to room temperature, the reaction mixture was poured into water (30 mL), and then it was extracted with EtOAc (30 mL×3). The combined organic layer was washed with brine (30 mL), dried over anhydrous Na₂SO₄, filtered and then concentrated. The residue was purified by Combi-Flash (8% EtOAc in PE) to give compound 29A (850 mg, yield 55.57%) as a white solid. ¹H NMR (CDCl₃, 400 MHz): δ 8.02-7.78 (m, 2H), 7.71-7.63 (m, 2H), 7.01-6.83 (m, 2H), 3.84 (s, 3H). MS (ESI) m/z (M+H)⁺ 175.9.

Intermediate 29A were subjected to conditions as described for compound 25 to yield compounds 29 and 30. The mixture was concentrated under vacuum. The product was purified by preparatory-HPLC (water (0.05% HC)-ACN). Compound 29 (64 mg, yield: 50%) as a yellow solid was obtained: ¹H NMR (DMSO-d₆, 400 MHz): δ 8.54 (s, 1H), 8.43 (s, 1H), 8.40 (brs, 3H), 7.74 (d, J=8.8 Hz, 2H), 7.32 (d, J=82.0 Hz, 1H), 7.08 (d, J=8.8 Hz, 2H), 4.69 (d, J=2.8 Hz 2H), 3.59 (d, J=4.8 Hz 2H). ¹⁹F NMR (376 MHz, DMSO-d₆): δ=−123.20. MS (ESI) m/z (M+H)⁺ 249.0.

### (Z)-3-fluoro-2-((4-(oxazol-4-yl)phenoxy)methyl)prop-2-en-1-amine hydrochloride (30)

Compound 30 (11.8 mg, yield: 9.1%) as a yellow solid was obtained: ¹H NMR (DMSO-d₆, 400 MHz): δ 8.54 (s, 1H), 8.43 (s, 1H), 8.30 (brs, 3H), 7.74 (d, J=8.8 Hz, 2H), 7.23 (d, J=82.4 Hz, 1H), 7.08 (d, J=8.8 Hz, 2H), 4.78 (d, J=2.4 Hz 2H), 3.53 (s, 2H). ¹⁹F NMR (376 MHz, DMSO-d₆): δ=−122.62 (s, 1F). MS (ESI) m/z (M+H)⁺ 248.9.

### (E)-3-fluoro-2-((thiophen-2-ylmethoxy)methyl)prop-2-en-1-amine (41)

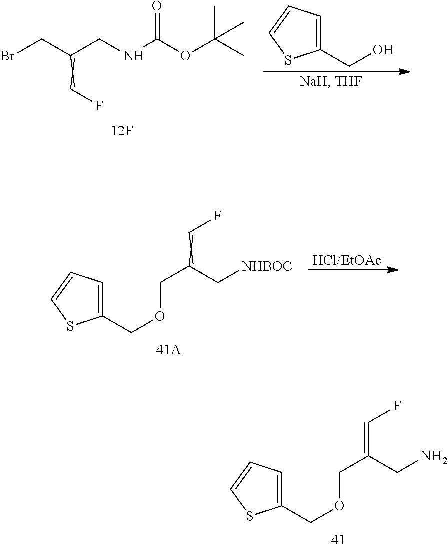

To a solution of NaH (112 mg, 2.80 mmol, 60% purity) in THF (10 mL) at 0° C. was added a solution of 2-thienylmethanol (319 mg, 2.80 mmol) in THF (2 mL) dropwise. After addition, the mixture was stirred at 0° C. for 30 min. Then a solution of intermediate 12F (500 mg, 1.86 mmol) in THF (3 mL) was added. The mixture was stirred at 20° C. for 1 h. The mixture was quenched with saturated NH₄Cl (10 mL), extracted with EtOAc (10 mL×2). The organics were collected and concentrated. The residue was purified by preparatory-HPLC (basic) to give compound 41A (200 mg, yield: 35.59%) as colorless oil. ¹H NMR (400 MHz, CDCl₃) δ 7.35-7.29 (m, 1H), 7.05-6.93 (m, 2H), 6.72 (s, 0.5H), 6.51 (s, 0.5H), 4.91-4.72 (m, 1H), 4.65 (s, 2H), 4.18-3.62 (m, 4H), 1.43 (s, 9H).

To a solution of compound 41A (60 mg, 199.09 umol) in EtOAc (3 mL) was added HCl/EtOAc (4M, 3 mL) dropwise. The mixture was stirred at 0° C. for 1 h. The solvent was removed by bubbling N2. The residue was dissolved in H₂O (1 mL), adjusted to pH 9 with NH₃.H₂O, diluted with CH₃CN (2 mL). The solution was purified by preparatory-HPLC (basic) to give compound 41 (20 mg, 4 yield: 9.42%) as white solid. MS (ESI) m/z (M+H)-202.1. ¹H NMR (400 MHz, CD₃CN) δ 7.42-7.36 (m, 1H), 7.07-7.04 (m, 1H), 7.03-6.98 (m, 1H), 6.80 (s, 0.5H), 6.58 (s, 0.5H), 4.65 (s, 2H), 4.05-3.97 (m, 2H), 3.49 (br. s, 2H).

### Example 14

### Compounds 21, 31-32, 47

### (E)-N-(2-(aminomethyl)-3-fluoroalkyl)benzamide hydrochloride (21)

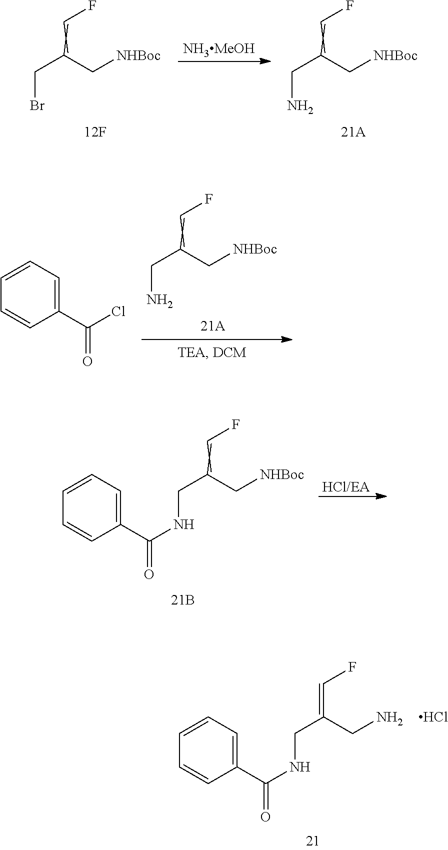

A mixture of intermediate 12F (200 mg, 745.93 umol) in NH₃.MeOH (7M, 2 mL) was stirred at 15° C. for 12 h. The mixture was concentrated. The residue was treated with TBME (3 mL). The insoluble substance was removed by filter. The filtrate was concentrated in vacuum to afford intermediate 21A (200 mg, crude) as pale yellow sticky oil, which was used for next step directly. ¹H NMR (DMSO-d₆, 400 MHz) δ 7.88 (br. s., 2H), 7.15-7.12 (m, 1H), 6.97 (d, J=82.8 Hz, 1H), 3.72-3.71 (m, 1H), 3.15-3.14 (m, 1H), 1.37 (s, 9H).

To a solution of intermediate 21A (200 mg, crude) and TEA (0.16 mL, 1.12 mmol) in DCM (5 mL) was added benzoyl chloride (105 mg, 745.93 umol). The mixture was stirred at 15° C. for 2 hours. The mixture was diluted with DCM (15 mL), washed with saturated NaHCO₃ (5 mL), brine (5 mL), dried over anhydrous MgSO₄, filtered and concentrated. The residue was purified by flash column chromatography over silica gel (petroleum ether: ethyl acetate=10:1 to 1:1) to afford compound 21B (140 mg, 49.73% yield over two steps) as colorless sticky oil. ¹H NMR (DMSO-d₆, 400 MHz) δ 8.45 (t, J=5.6 Hz, 1H), 7.80-7.80 (m, 2H), 7.51-7.42 (m, 3H), 694-6.93 (m, 1H), 6.78 (d, J=84.8 Hz, 1H), 3.79-3.77 (m, 2H), 3.69-3.68 (m, 2H), 1.32 (s, 9H). MS (ESI) m/z (M+Na)⁺ 332.2.

HCl/EtOAc (4M, 2 mL, 8.0 mmol) was added to a solution of compound 21B (0.14 g, 454.03 umol) in EA (2 mL) at 0° C. The mixture was stirred for 2 hours at 0° C. The mixture was concentrated. The residue) was purified by preparatory-HPLC (column: YMC-Actus Tri-art C18 150*30 mm*5 um; mobile phase: [water (0.05% HCl)-ACN]; B %: 0%-25%, 8 min) to afford compound 21 (50 mg, 52.8% yield) as colorless sticky oil. ¹H NMR (DMSO-d₆, 400 MHz) δ 9.04 (br t, J=5.6 Hz, 1H), 8.34 (br. s., 3H), 7.97-7.89 (m, 2H), 7.58-7.52 (m, 1H), 7.50-7.44 (m, 2H), 7.10 (d, J=83.2 Hz, 1H), 4.00-3.92 (m, 2H), 3.53 (br. d., J=4.8 Hz, 2H). ¹⁹F NMR (DMSO-d₆, 376 MHz): −126.45. MS (ESI) m/z (M+H)⁺ 209.1.

### (E)-N-(2-(aminomethyl)-3-fluoroalkyl)benzamide hydrochloride (31)

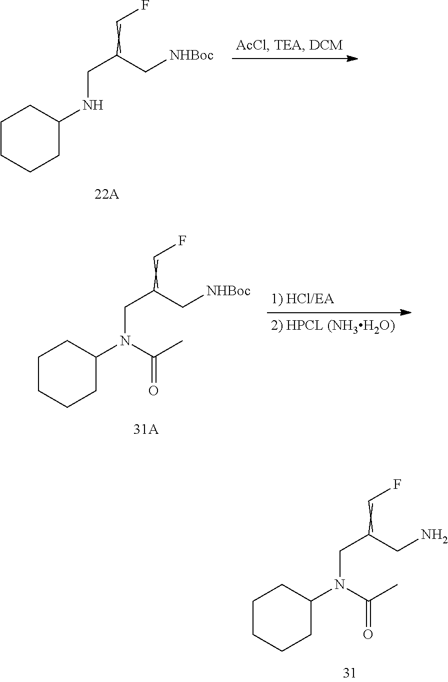 [SMILES: CC(C)(C)OC(=O)NCC(=CF)CNC1CCCCC1 ; CC(=O)N(CC(=CF)CNC(=O)OC(C)(C)C)C1CCCCC1 ; CC(=O)N(CC(=CF)CN)C1CCCCC1]

Intermediate 22A was treated with acetyl chloride using conditions as for intermediate 21B to yield intermediate 31A (150 mg, 71.9% yield) as colorless oil, ¹H NMR (CDCl₃, 400 MHz): δ 6.57-6.32 (m, 1H), 5.68 (s, 0.7H), 4.74 (s, 0.3H), 4.42-4.26 (m, 0.3H), 3.97-3.84 (m, 2H), 3.83-3.68 (m, 2H), 2.16 (s, 2.1H), 2.07 (s, 0.9H), 1.90-1.69 (m, 6H), 1.54-1.33 (m, 12H), 1.18-0.98 (m, 1H). MS (ESI) m/z (M+H)⁺ 329.1

To a solution of intermediate 21A (200 mg, crude) and TEA (0.16 mL, 1.12 mmol) in DCM (5 mL) was added benzoyl chloride (105 mg, 745.93 umol). The mixture was stirred at 15° C. for 2 hours. The mixture was diluted with DCM (15 mL), washed with saturated NaHCO₃ (5 mL), brine (5 mL), dried over anhydrous MgSO₄, filtered and concentrated. The residue was purified by flash column chromatography over silica gel (petroleum ether: ethyl acetate=10:1 to 1:1) to afford compound 21B (140 mg, 49.73% yield over two steps) as colorless sticky oil. ¹H NMR (DMSO-d₆, 400 MHz) δ 8.45 (t, J=5.6 Hz, 1H), 7.80-7.80 (m, 2H), 7.51-7.42 (m, 3H), 694-6.93 (m, 1H), 6.78 (d, J=84.8 Hz, 1H), 3.79-3.77 (m, 2H), 3.69-3.68 (m, 2H), 1.32 (s, 9H). MS (ESI) m/z (M+Na)⁺ 332.2.

HCl/EtOAc (4M, 2 mL, 8.0 mmol) was added to a solution of compound 31A (0.15 g, 457 umol) in EA (2 mL) at 0° C. The mixture was stirred for 2 hours at 20° C. The mixture was concentrated. The residue was purified by preparatory-HPLC [water (0.05% ammonia hydroxide v/v)-ACN]; B %: 0%-25%, 8 min) to afford compound 31 (25.6 mg, 23.81% yield) as yellow oil. ¹H NMR (CDCl₃, 400 MHz): δ 6.74-6.45 (m, 1H), 4.02-3.90 (m, 2.3H), 3.59-3.53 (m, 0.7H), 3.18-3.17 (m, 0.9H), 3.05-3.04 (m, 1.OH), 2.10 (s, 2H), 2.08 (s, 1H), 1.72-1.67 (m, 3H), 1.55-1.45 (m, 4H), 1.31-1.27 (m, 2H), 1.21-1.07 (m, 1H). ¹⁹F NMR (376 MHz, DMSO-d₆): δ−137.70, −139.93. MS (ESI) m/z (M+H)⁺ 229.0.

### N-(2-(aminomethyl)-3-fluoroalkyl)-N-phenylacetamide (32)

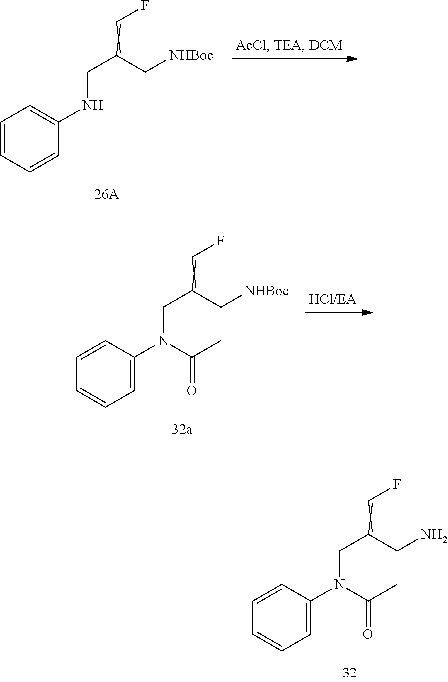 [SMILES: CC(C)(C)OC(=O)NCC(=CF)CNc1ccccc1 ; CC(=O)N(CC(=CF)CNC(=O)OC(C)(C)C)c1ccccc1 ; CC(=O)N(CC(=CF)CN)c1ccccc1]

Compound 32 was prepared from intermediate 26A using the procedures as for compound 31. The crude product was purified by preparatory-HPLC [water (0.05% HCl)-ACN] to afford compound 32 (E:Z=3:1; 25 mg, 23.2% yield) as yellow oil. ¹H NMR (400 MHz, DMSO-d₆) δ 8.39 (br. s., 1H), 8.18 (br. s., 2H), 7.46-7.27 (m, 3H), 7.22 (d, J=7.6 Hz, 2H), 7.14-6.74 (m, 1H), 4.55-4.41 (m, 2H), 4.07 (br. s., 1H), 3.96 (d, J=2.0 Hz, 1H), 3.42 (d, J=4.8 Hz, 2H), 3.34 (d, J=4.0 Hz, 1H), 2.13 (s, 1H), 2.06 (s, 2H). ¹⁹F NMR (376 MHz, DMSO-d₆) δ −125.38 (s, 1F), −127.75 (s, 1F). MS (ESI) m/z (M+H)⁺ 237.2.

### phenyl (E)-(2-(aminomethyl)-3-fluoroalkyl)carbamate hydrochloride (47)

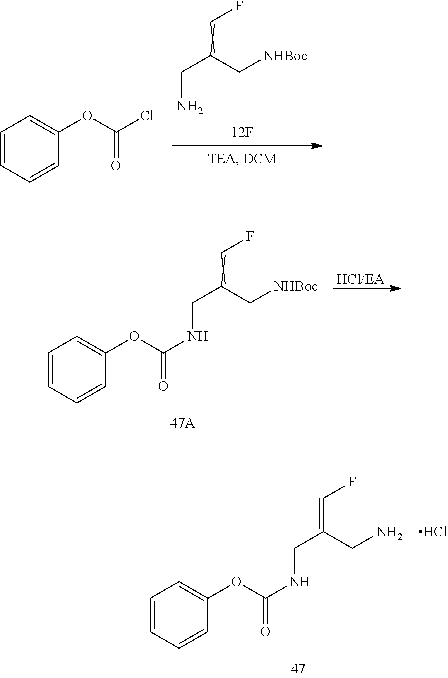

Compound 47 was prepared from phenyl carbonochloridate and intermediate 12F using the procedures as for compound 31. The crude product was purified by preparatory-HPLC [water (0.05% HCl)-ACN] to afford compound 47 (20 mg, 23.9% yield) as pale yellow oil. ¹H NMR (DMSO-d₆, 400 MHz) δ 8.30 (br. s., 3H), 8.11-8.03 (m, 1H), 7.43-7.32 (m, 2H), 7.24-7.18 (m, 1H), 7.12 (d, J=7.6 Hz, 2H), 7.05 (d, J=83.2 Hz, 1H), 3.78 (d, J=3.6 Hz, 2H), 3.54 (br. s., 2H). ¹⁹F NMR (DMSO-d₆, 376 MHz) δ−126.30. MS (ESI) m/z (M+Na)⁺ 225.1.

### Example 15

### Compound 15

### (E)-2-((1H-indol-1-yl)methyl)-3-fluoroprop-2-en-1-amine hydrochloride (15)

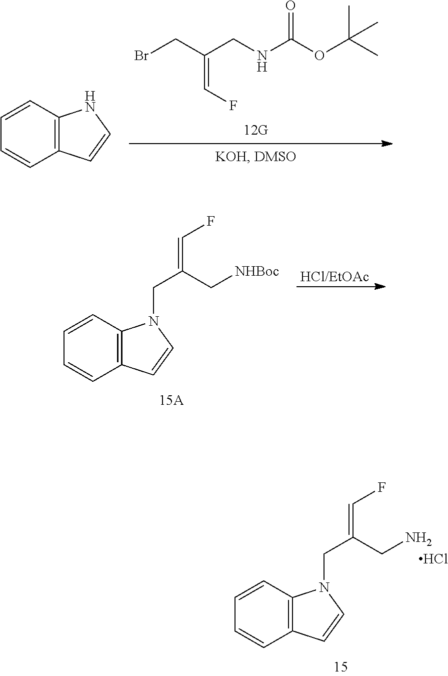 [SMILES: c1ccc2[nH]ccc2c1 ; CC(C)(C)OC(=O)NC/C(=C\F)CBr ; CC(C)(C)OC(=O)NC/C(=C\F)Cn1ccc2ccccc21 ; "HCl/EtOAc" ; NC/C(=C\F)Cn1ccc2ccccc21 ; Cl]

To a solution of indole (90 mg, 768.26 umol) and compound 12G (206 mg, 768.26 umol) in DMSO (5 mL) was added KOH (86 mg, 1.54 mmol). The mixture was stirred at 20° C. for 1 h. The mixture was washed with H₂O (15 mL), extracted with EtOAc (15 mL×2). The organics were collected and concentrated. The residue was purified by column (PE:EA=1:0-5:1) to give compound 15A (83 mg, yield: 32.94%) as white solid. MS (ESI) m/z (M+H)⁺ 304.9.

To a solution of compound 15A (83 mg, 272.70 umol) in EtOAc (2 mL) was added HCl/EtOAc (4M, 3.9 mL). The mixture was stirred at 20° C. for 30 min. The mixture was continued stirring for 1.5 h. The solvent was removed in vacuo. The residue was purified by preparatory-HPLC (Neutral) to give compound 15 (8 mg, yield: 11.87%) as colorless oil. MS (ESI) m/z (M+H)⁺ 204.9. ¹H NMR (400 MHz, CD₃OD) δ 7.53 (d, J=7.8 Hz, 1H), 7.43 (d, J=8.0 Hz, 1H), 7.26-7.20 (m, 1H), 7.17-7.10 (m, 1H), 7.06-6.98 (m, 1H), 6.83 (s, 1H), 6.62 (s, 1H), 6.48-6.43 (m, 1H), 4.80-4.75 (m, 2H), 3.15-3.08 (m, 2H).

### Example 16

### Compounds 57, 58

### (E)-4-(2-(2-(aminomethyl)-3-fluoroalkyl)-2H-1,2,3-triazol-4-yl)phenol hydrochloride (57)

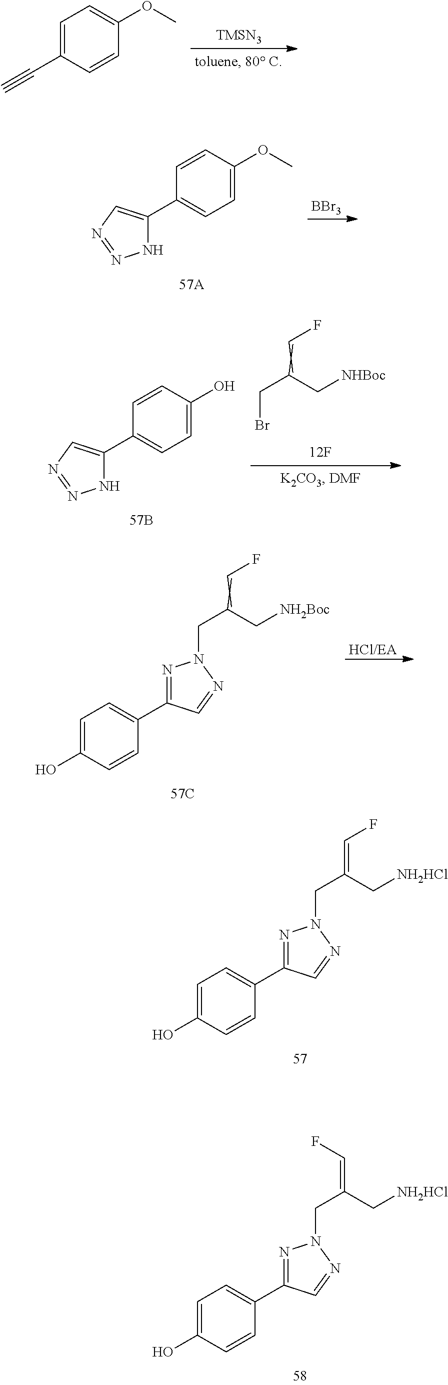

The mixture of 1-ethynyl-4-methoxybenzene (3 g, 22.70 mmol), TMSN₃ (4.97 g, 43.13 mmol) in Toluene (23 mL) was stirred at 110° C. for 72 h. The mixture was added H₂O (30 mL) and extracted with EA (30 mL), the organic phase was washed with brine (20 mL), dried over Na₂SO₄, filtered and concentrated under vacuum. The product was purified by Flash Column Chromatography (0-5% MeOH/DCM). Compound 57A (800 mg, yield 20.12%) was obtained as a gray solid. ¹H NMR (DMSO-d₆, 400 MHz) δ 14.82 (br. s., 1H), 8.15 (s, 1H), 7.79 (br d, J=8.5 Hz, 2H), 7.02 (br d, J=8.3 Hz, 2H), 3.80 (s, 3H).

BBr₃ (0.4 mL, 3.42 mmol) was added to the mixture of compound 57A (200 mg, 1.14 mmol) in DCM (10 mL). The mixture was stirred at −78° C. for 1 h. Then the mixture was stirred at 20° C. for 21 h. The mixture was quenched by ice water (20 mL) and extracted with EA (30 mL). The organic phase was dried over Na₂SO₄, filtered and concentrated under vacuum. The product was purified by Flash Column Chromatography (0-100% EA/PE). Compound 57B (100 mg, yield 42.39%) was obtained as a yellow solid.

Compound 57B was subjected to treatment with intermediate 12F using conditions as described for compound 7 to yield the intermediate 57C. HCl/EtOAc (4M, 1.11 mL) was added to the mixture of compound 57C (155 mg, 444.93 umol) in EA (5 mL). The mixture was stirred at 20° C. for 1 h. The mixture was concentrated under vacuum. The product was purified by preparatory-HPLC (water (0.05% HCl)-ACN). Compound 57 (30 mg, yield 22.9%) was obtained as a yellow solid was obtained: ¹H NMR (CD₃OD, 400 MHz): δ 7.99 (s, 1H), 7.71-7.65 (m, 2H), 7.41-7.18 (m, 1H), 6.89-6.84 (m, 2H), 5.17 (d, J=2.8 Hz, 2H), 3.74 (s, 2H). MS (ESI) m/z (M+H)⁺ 249.1.

### (Z)-4-(2-(2-(aminomethyl)-3-fluoroalkyl)-2H-1,2,3-triazol-4-yl)phenol hydrochloride (58)

Compound 58 (8.5 mg, yield 4.97%) was obtained as a yellow solid was obtained. ¹H NMR (CD₃OD, 400 MHz): δ 7.98 (s, 1H), 7.68 (d, J=8.5 Hz, 2H), 7.28-7.04 (m, 1H), 6.87 (d, J=8.5 Hz, 2H), 5.33 (d, J=2.0 Hz, 2H), 3.62 (br s, 2H). MS (ESI) m/z (M+H)⁺ 249.1.

### Example 17

### Compounds 59, 69, and 70

### (E)-1-(2-(aminomethyl)-3-fluoroalkyl)-N-(tert-butyl)-1H-indole-5-carboxamide (59)

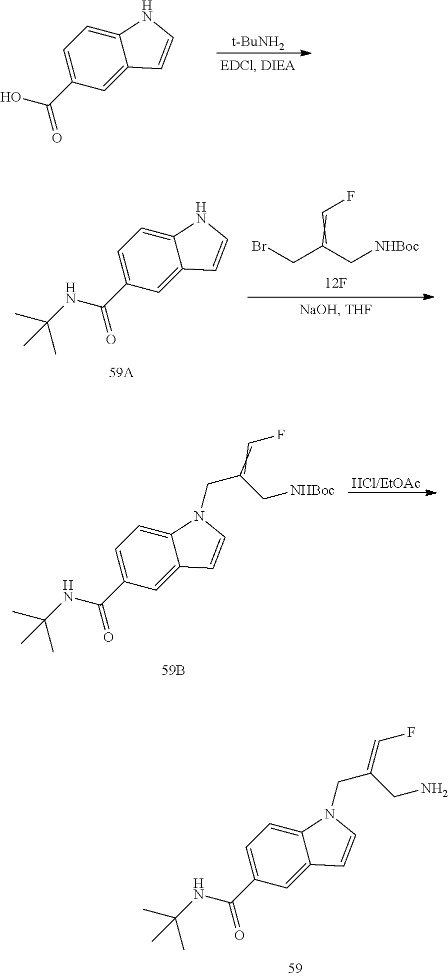

To a solution of 1H-indole-5-carboxylic acid (200 mg, 1.24 mmol) in DCM (6 mL) was added t-BuNH₂ (99.8 mg, 1.37 mmol), DIEA (160 mg, 1.24 mmol) and EDCI (262 mg, 1.37 mmol). The mixture was stirred at 20° C. for 12 h. The mixture was dissolved in DCM (30 mL) and washed with 0.5N HCl solution (20 mL), saturated NaHCO₃ solution (20 mL) and saturated brine (20 mL), dried over Na₂SO₄, filtered and concentrated under reduced pressure to give a residue. The residue was purified by preparatory-HPLC (water (0.05% HCl)-ACN) to afford compound 59A (320 mg, average yield 39.4%) as a white solid. ¹H NMR (400 MHz, DMSO-d₆) δ 11.25 (br. s., 1H), 8.07 (s, 1H), 7.59-7.53 (m, 2H), 7.42-7.35 (m, 2H), 6.50 (br. s., 1H), 1.39 (s, 9H).

To a mixture of compound 59A (150 mg, 694 umol) and intermediate 12F (186 mg, 694 umol) in THF (4 mL) was added NaOH (155 mg, 3.88 mmol). The mixture was stirred 40° C. for 4 hours. The reaction mixture was diluted with H₂O (20 mL) and extracted with EA (25 mL×2). The combined organic layers were washed with saturated brine (25 mL), dried over Na₂SO₄, filtered and concentrated under reduced pressure to give a residue. The residue was purified by column chromatography (SiO₂, Petroleum ether: Ethyl acetate 1:0 to 1:1) to afford the product (200 mg), which was still impure. The impure product was purified by preparatory-HPLC (water (0.05% ammonia hydroxide v/v)-ACN) to afford compound 59B (95 mg, yield 25.4%) as a white solid. ¹H NMR (400 MHz, DMSO-d₆) δ 8.06 (d, J=1.2 Hz, 1H), 7.63-7.40 (m, 4H), 7.20 (br. s., 1H), 6.99 (d, J=83.6 Hz, 1H), 6.57-6.50 (m, 1H), 4.74 (br. s., 2H), 3.48 (d, J=4.4 Hz, 2H), 1.39 (s, 18H). ¹⁹F NMR (376 MHz, DMSO-d₆) δ−130.97. MS (ESI) m/z (M-t-Bu+H)⁺ 348.2.

HCl/EtOAc (4.00 M, 0.59 mL) was added to compound 59B (95.0 mg, 235 umol). The mixture was stirred at 20° C. for 1 h. The reaction mixture was concentrated under reduced pressure to remove solvent. The residue was purified by preparatory-HPLC (water (10 mM NH₄HCO₃)-ACN) to afford the product compound 59 (20.0 mg, yield 28.0%) as a white solid. ¹H NMR (400 MHz, DMSO-d₆) δ 8.06 (d, J=0.8 Hz, 1H), 7.63-7.53 (m, 3H), 7.47 (d, J=3.2 Hz, 1H), 6.96 (d, J=85.2 Hz, 1H), 6.54 (d, J=3.2 Hz, 1H), 4.84 (d, J=2.0 Hz, 2H), 2.95 (d, J=2.0 Hz, 2H), 1.39 (s, 9H). ¹⁹F NMR (376 MHz, DMSO-d₆) δ −134.02. MS (ESI) m/z (M+H)⁺ 304.2.

### 1-(2-(aminomethyl)-3-fluoroalkyl)-N-(tert-butyl)-1H-indole-4-carboxamide (69)

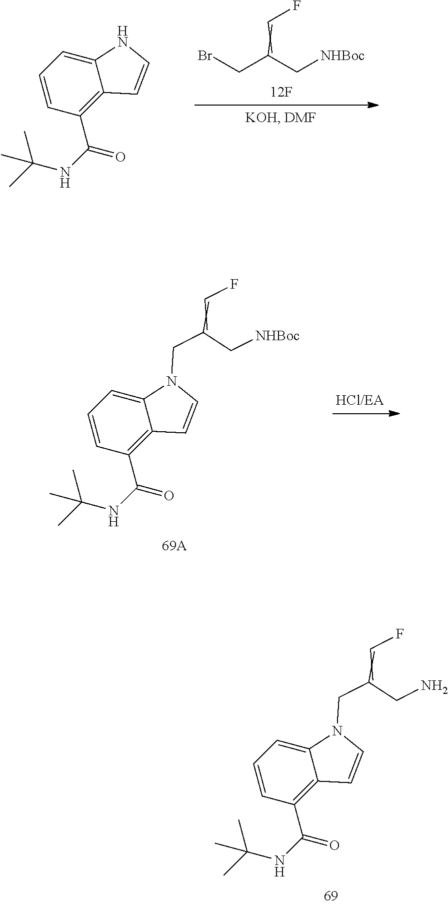 [SMILES: CC(C)(C)NC(=O)c1cccc2[nH]ccc12 ; CC(C)(C)OC(=O)NCC(=CF)CBr ; CC(C)(C)NC(=O)c1cccc2c1ccn2CC(=CF)CNC(=O)OC(C)(C)C ; CC(C)(C)NC(=O)c1cccc2c1ccn2CC(=CF)CN]

Intermediate N-(tert-butyl)-1H-indole-4-carboxamide was treated with intermediate 12F using conditions as described for compound 59 t obtain the intermediate 69A. Compound 69A (70.0 mg, 42.5% yield) was obtained as a yellow solid.

To a solution of compound 2 (65.0 mg, 161.09 umol) in EtOAc (2 mL) was added HCl/EtOAc (4 M, 6 mL) dropwise. The solution was stirred at 25° C. for 40 min. The solvent was removed in vacuo. The residue was purified by preparatory-HPLC (basic). Compound 69 (35.0 mg, 70.9% yield, 99% purity) was obtained as a yellow oil. ¹H NMR (400 MHz, DMSO-d₆) 7.63 (d, J=8.0 Hz, 1H), 7.53-7.46 (m, 1H), 7.45-7.39 (m, 1H), 7.33-7.27 (m, 1H), 7.16-7.09 (m, 1H), 7.06-6.94 (m, 0.5H), 6.85-6.73 (m, 0.5H), 6.72-6.67 (m, 1H), 4.96-4.87 (m, 0.5H), 4.86-4.71 (m, 1.5H), 2.96-2.88 (m, 1.3H), 2.75-2.69 (m, 0.6H), 1.37 (s, 9H). MS (ESI) m/z (M+H)⁺ 304.1.

### 1-(2-(aminomethyl)-3-fluoroalkyl)-N-(tert-butyl)-1H-indole-3-carboxamide (70)

1H-indole-3-carboxylic acid was subjected to same conditions as described for compound 59 to obtain the target compound 70. Compound 70 (35.0 mg, 70.9% yield, 99% purity) was obtained as a yellow oil. ¹H NMR (400 MHz, DMSO-d₆) 8.18-8.11 (m, 1H), 8.08 (s, 1H), 7.59 (d, J=8.4 Hz, 0.7H), 7.44 (d, J=8.0 Hz, 0.3H), 7.27-7.11 (m, 3H), 7.10-7.04 (m, 0.4H), 6.91 (br. s, 0.3H), 6.84 (br. s, 0.2H), 4.96-4.90 (m, 0.6H), 4.85-4.75 (m, 1.3H), 3.07-2.97 (m, 1.4H), 2.87-2.80 (m, 0.6H), 1.40 (s, 9H). MS (ESI) m/z (M+H)⁺ 304.2.

### Example 18

### Compound 61

### (E)-2-((4-(4,5-dihydro-1H-imidazol-2-yl)phenoxy)methyl)-3-fluoroprop-2-en-1-amine (61)

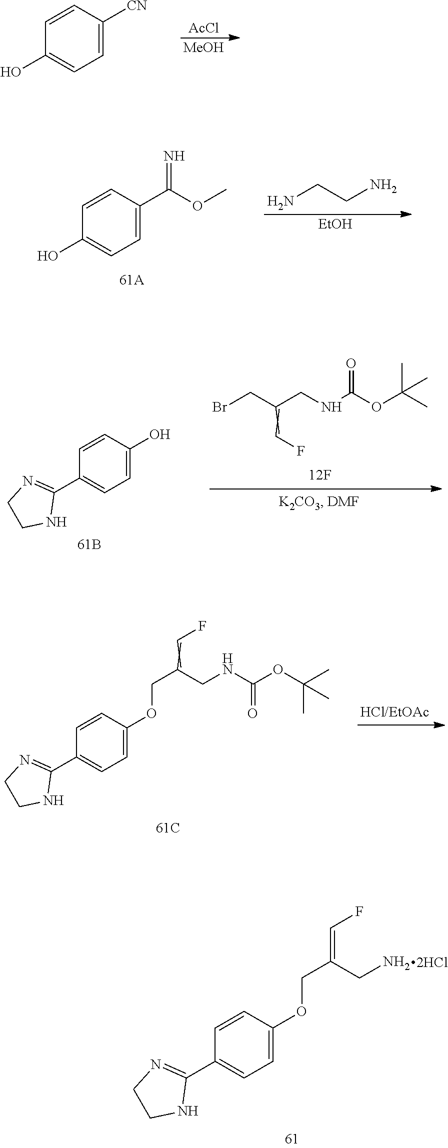

To a solution of 4-hydroxybenzonitrile (2 g, 16.79 mmol) in MeOH (8 mL) was added acetyl chloride (134.32 mmol, 9.6 mL) dropwise. The mixture was stirred at 20° C. for 12 h. The solid was filtered, washed with EtOAc (10 mL). The solid was filtered, collected and dried in vacuo. Compound 61A (3.07 g, crude, HCl) was obtained as white solid, which was used directly for the next step without further purification.

The solution of compound 61A (3.07 g, 16.36 mmol, HCl) and ethane-1,2-diamine (32.73 mmol, 2.2 mL) in EtOH (20 mL) was stirred at 70° C. for 12 h. The solvent was removed in vacuo. The residue was triturated with EtOH (5 mL). The solid was filtered, collected and dried in vacuo. Compound 61B (1 g, 37.7% yield) was obtained as white solid.

To a solution of compound 61B (150.0 mg, 924.85 umol) and tert-butyl N-[2-(bromomethyl)-3-fluoro-allyl]carbamate 12F (248.0 mg, 924.85 umol) in DMF (5 mL) was added K₂CO₃ (383.5 mg, 2.77 mmol). The mixture was stirred at 25° C. for 12 h. The mixture was diluted with EtOAc (30 mL), washed with 1N NaOH (15 mL×2). The organics were collected, washed with brine (15 mL×3), dried with Na₂SO₄, filtered and concentrated. The crude was triturated with EtOAc (30 mL). The solid was filtered, collected and dried in vacuo. Compound 61C (100.0 mg, 30.9% yield) as was obtained as white solid. ¹H NMR (400 MHz, DMSO-d₆) δ 7.74-7.68 (m, 2H), 7.12 (s, 1H), 7.06 (br s, 1H), 6.98-6.87 (m, 3H), 4.46-4.40 (m, 2H), 3.75-3.67 (m, 2H), 3.53 (s, 4H), 1.30 (s, 9H).

To a solution of t compound 61C (100 mg, 286.21 umol) in EtOAc (2 mL) was added HCl/EtOAc (4M, 5 mL). The mixture was stirred at 25° C. for 2 h. The solvent was removed in vacuo. Compound 61 (70.0 mg, 42.2% yield, 98.5% purity, HCl) was obtained as white solid. ¹H NMR (400 MHz, DMSO-d₆) 10.61 (br. s, 2H), 8.36 (br. s, 3H), 8.04 (d, J=9.0 Hz, 2H), 7.42 (s, 0.5H), 7.26-7.16 (m, 3H), 4.87-4.66 (m, 2H), 4.05-3.83 (m, 4H), 3.68-3.47 (m, 2H). MS (ESI) m/z (M+H)⁺ 250.0.

### Example 19

### Compounds 64, 60, and 63

### (E)-2-((4-(1H-1,2,4-triazol-5-yl)phenoxy)methyl)-3-fluoroprop-2-en-1-amine (64)

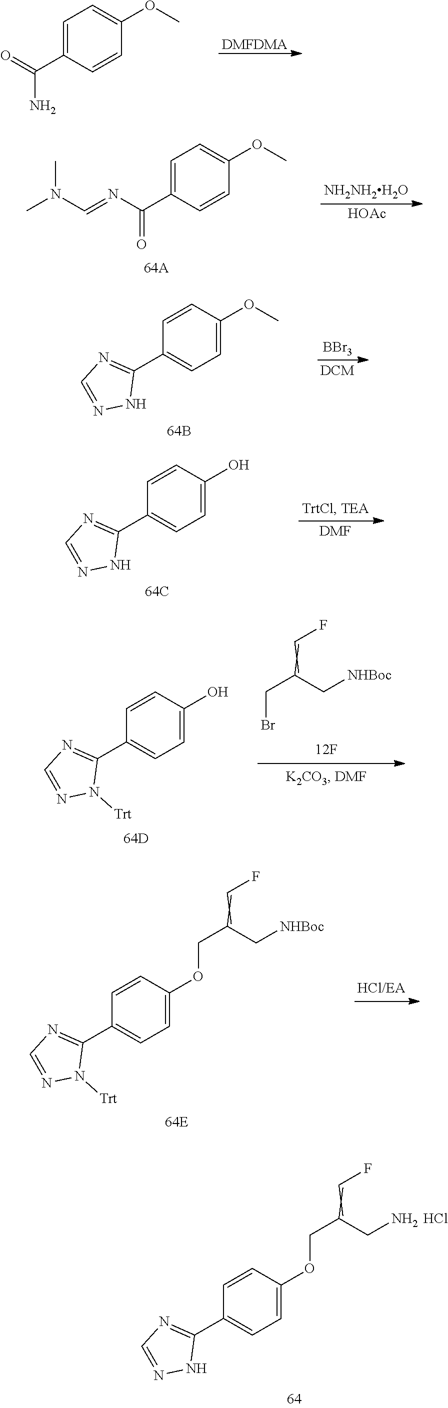

A mixture of 4-methoxybenzamide (5 g, 33.08 mmol) and DMF/DMA (13.18 mL, 99.23 mmol) was heated to 120° C. and stirred for 1.5 h. The mixture was concentrated in vacuum to afford white solid. The resultant solid was triturated with TBME:EA (10:1, 10 mL). The precipitate was collected by filter and dried in vacuum to afford compound 64A (6 g, yield 88.0%) as white solid. ¹H NMR (DMSO-d₆, 400 MHz): δ 8.57 (s, 1H), 8.11 (d, J=8.8 Hz, 2H), 6.97 (d, J=8.8 Hz, 2H), 3.81 (s, 3H), 3.21-3.15 (m, 3H), 3.12 (s, 3H).

A mixture of compound 64A (6.00 g, 29.09 mmol) and NH₂NH₂.H₂O (4.00 mL, 85% purity, 69.96 mmol) in HOAc (100 mL) was heated to 100° C. and stirred for 2 hours. The mixture was concentrated in vacuum. The residue was treated with DCM (75 mL) and H₂O (75 mL). The insoluble substance was collected by fitter, dried in vacuum to afford compound 64B (3 g, 58.9% yield) as white solid, which was used for next step directly.

¹H NMR (DMSO-d₆, 400 MHz): δ 8.30 (s, 1H), 7.95 (d, J=8.8 Hz, 2H), 7.05 (d, J=8.8 Hz, 2H), 3.81 (s, 3H).

BBr₃ (2.15 g, 8.56 mmol) was added to the mixture of compound 64B (500 mg, 2.85 mmol) in DCM (15 mL) at −78° C. The mixture was stirred at 50° C. for 24 h. The mixture was concentrated under vacuum and added H₂O (15 mL), the pH of the mixture was adjusted to 6-7 by saturated NaHCO₃, then filtered. The filter cake was dried under vacuum. Compound 64C (300 mg, yield 62.16%) was obtained as a brown solid, which was used for next step without purification. ¹H NMR (METHANOL-d₄, 400 MHz): δ 9.68 (s, 1H), 8.60 (br s, 1H), 7.85 (d, J=8.8 Hz, 2H), 6.90 (d, J=8.8 Hz, 2H). MS (ESI) m/z (M+H)⁺ 162.1.

TrtCl (381 mg, 1.37 mmol) was added to the mixture of compound 64C (200 mg, 1.24 mmol) and TEA (0.3 mL, 2.11 mmol) in DMF (5 mL) at 0° C. Then the mixture was stirred at 20° C. for 18 h. The mixture was concentrated under vacuum and added H₂O (40 mL), extracted with EA (20 mL). The organic phase was washed with brine (10 mL), dried over Na₂SO₄, filtered and concentrated under vacuum. The product was purified by Flash Column Chromatography (0-50% EA/PE). Compound 64D (186 mg, yield 37.15%) was obtained as a white oil. ¹H NMR (CD₃OD, 400 MHz): δ 8.08 (s, 1H), 7.87-7.82 (m, 2H), 7.42-7.38 (m, 9H), 7.21 (dd, J=3.0, 6.8 Hz, 6H), 6.86-6.82 (m, 2H).

K₂CO₃ (95 mg, 688.12 umol) was added to the mixture of compound 12F (123 mg, 458.75 umol) and compound 64D (185 mg, 458.75 umol) in DMF (5 mL) at 20° C. for 19 h. The mixture was added H₂O (50 mL) and extracted with EA (30 mL), the organic phase was washed with 1M NaOH (20 mL), brine (20 mL) and dried over Na₂SO₄, filtered and concentrated under vacuum. Compound 64E (270 mg, crude) was obtained as a yellow oil. MS (ESI) m/z (M+H)⁺ 591.3.

HCl/EtOAc (4M, 1.14 mL) was added to the mixture of Compound 64E (270 mg, 457.10 umol) in EA (5 mL) at 20° C. for 1.5 h. The mixture was concentrated under vacuum. The product was purified by preparatory-HPLC (water (0.05% HCl)-ACN). Compound 64 was obtained as a pale-yellow solid. ¹H NMR (DMSO-d₆, 400 MHz): δ 8.63 (br. s., 1H), 8.31 (br. s., 3H), 8.00 (d, J=8.6 Hz, 2H), 7.43-7.18 (m, 1H), 7.12 (d, J=8.8 Hz, 2H), 4.69 (d, J=2.9 Hz, 2H), 3.57 (br d, J=4.4 Hz, 2H). MS (ESI) m/z (M+H)⁺ 249.1.

### (2-((4-(1H-1,2,3-triazol-5-yl)phenoxy)methyl)-3-fluoroprop-2-en-1-amine (60)

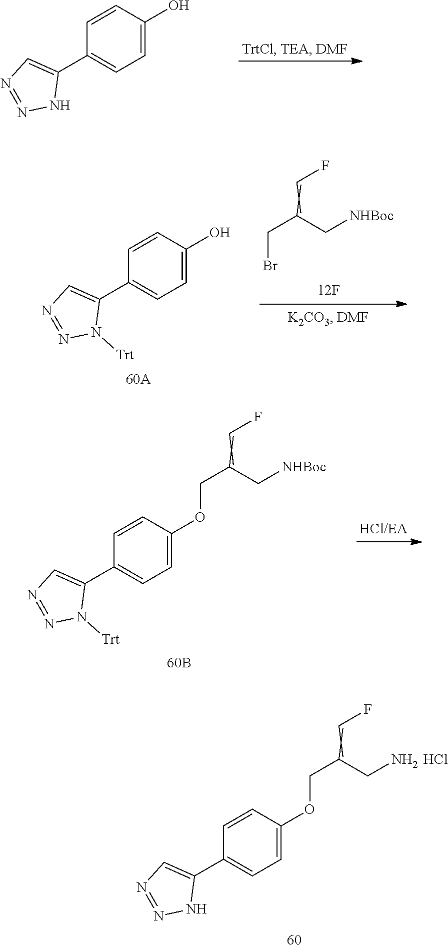

4-(1H-1,2,3-triazol-5-yl)phenol was subjected to treatment with trityl chloride (TrtCl) and the resulting intermediate 60A was treated with intermediate 12F using conditions as described for compound 61. The final compound was purified by preparatory-HPLC (water (0.05% HCl)-ACN). Compound 60 (7 mg, yield 15.85%) was obtained as yellow oil. ¹H NMR (CD₃OD, 400 MHz): δ 8.32 (s, 1H), 7.99 (s, 0.5H), 7.85-7.80 (m, 2H), 7.70-7.66 (m, 1H), 7.40-7.36 (m, 0.7H), 7.20-7.14 (m, 3H), 6.87 (d, J=8.8 Hz, 1H), 5.17 (d, J=2.5 Hz, 1H), 4.70 (d, J=3.3 Hz, 2H), 3.86 (s, 2H), 3.74 (br s, 1H). MS (ESI) m/z (M+H)⁺ 249.1.

### (Z)-2-((4-(1H-imidazol-2-yl)phenoxy)methyl)-3-fluoroprop-2-en-1-amine (63)

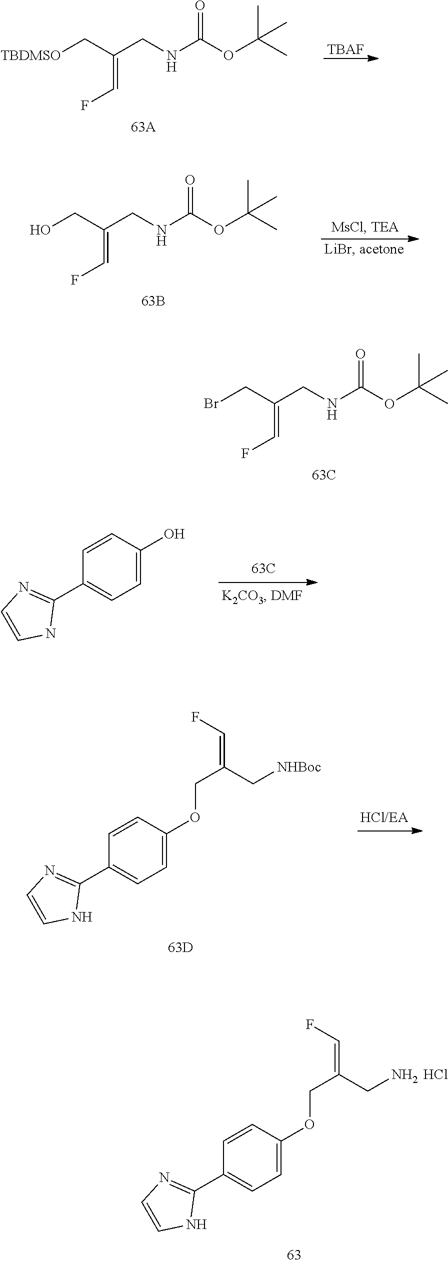

TBAF (1M, 0.7 mL) was added to the mixture of compound 63A (190 mg, 594.71 umol) in THF (5 mL) at 20° C. for 1 h. The mixture was added H₂O (10 mL) and extracted with EA (30 mL), the organic phase was washed with brine (15 mL), dried over Na₂SO₄, filtered and concentrated under vacuum. The product was purified by Flash Column Chromatography (0-50% EA/PE). Compound 63B (122 mg, crude) was obtained as a yellow oil.

TEA (0.1 mL, 891.70 umol) was added to the mixture of compound 63B (122 mg, 594.47 umol) in Acetone (5 mL). Then MsCl (0.05 mL, 713.36 umol) was added dropwise to the mixture and the mixture was stirred at 0° C. for 1 h. The mixture was filtered and LiBr (258 mg, 2.97 mmol) was added to the filtrate and the mixture was stirred at 20° C. for 1 h. The mixture was added H₂O (50 mL) and extracted with EA (30 mL), the organic phase was washed with brine (20 mL), dried over Na₂SO₄, filtered and concentrated under vacuum. Compound 63C (160 mg, crude) was obtained as a yellow oil, which was used for next step without purification.

4-(1H-imidazol-2-yl)phenol and intermediate 63C were subjected to same conditions as described in the synthesis of compound 60. The resulting intermediate 63D was then further treated with HCl/EtOAc and purified the resulting product by preparatory-HPLC (water (0.05% HCl)-ACN). Compound 63 (10 mg, yield 7.37%, HCl) was obtained as a white solid. ¹H NMR (CD₃OD, 400 MHz): δ 7.95-7.90 (m, 2H), 7.60 (s, 2H), 7.31 (d, J=9.0 Hz, 2H), 7.16 (d, J=80.4 Hz, 1H), 4.94 (d, J=2.5 Hz, 2H), 3.73 (br s, 2H). MS (ESI) m/z (M+H)⁺ 248.1.

### Example 20

### Compounds 65-68 and 71-72

### (E)-1-(4-((2-(aminomethyl)-3-fluoroalkyl)amino)phenyl)-3-ethyl-1-methylurea (66) and

### (Z)-1-(4-((2-(aminomethyl)-3-fluoroalkyl)amino)phenyl)-3-ethyl-1-methylurea (67)

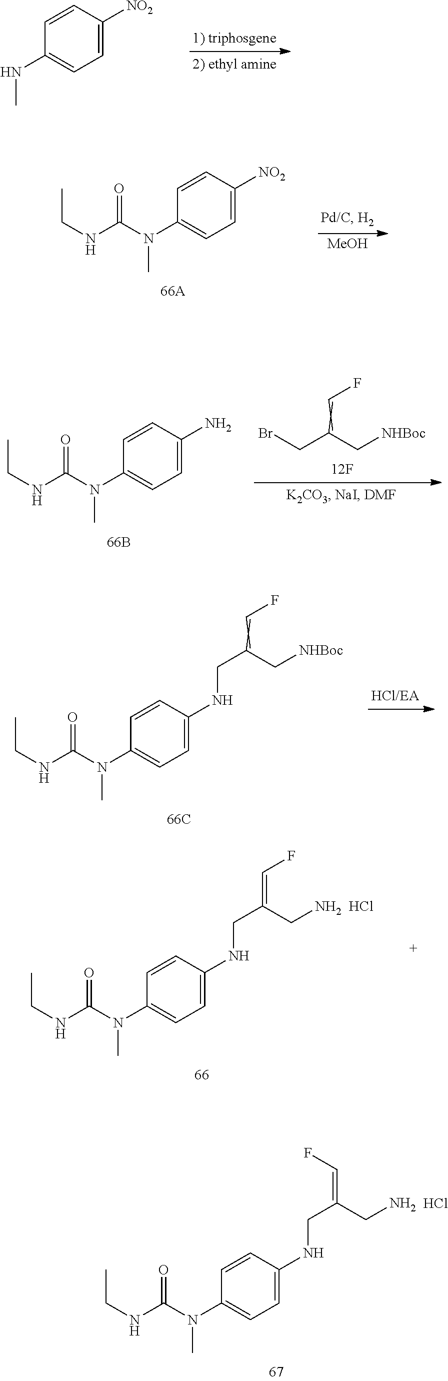

To a mixture of N-methyl-4-nitroaniline (500 mg, 3.29 mmol) and Et₃N (665 mg, 6.57 mmol) in toluene (4 mL) was added triphosgene (341 mg, 1.15 mmol) dissolved in toluene (1 mL) dropwise. After the reaction mixture was stirred at 20° C. for 0.5 h, white solid precipitates were removed by filtration through a pad of celite. Ethylamine (148 mg, 3.29 mmol) was added to the filtrate and then the mixture was stirred at 20° C. for 1 h. The reaction mixture was concentrated under reduced pressure to remove solvent to give a residue. The residue was purified by column chromatography (SiO₂, petroleum ether/ethyl acetate 1:0 to 1:1) to afford compound 66A (480 mg, yield 65.4%) as a yellow solid. ¹H NMR (400 MHz, DMSO-d₆) δ 8.20-8.13 (m, 2H), 7.53-7.47 (m, 2H), 7.04 (t, J=5.2 Hz, 1H), 3.26 (s, 3H), 3.16-3.08 (m, 2H), 1.06 (t, J=6.8 Hz, 3H).

To a solution of compound 66A (480 mg, 2.15 mmol) in MeOH (10 mL) was added Pd/C (100 mg, 10% purity) under N2. The suspension was degassed under vacuum and purged with H₂ several times. The mixture was stirred under H₂ (15 psi) at 20° C. for 2 hours. The mixture was filtered and concentrated under reduced pressure to give a crude product 66B (440 mg, crude) as a yellow oil, which was used for next step without purification. ¹H NMR (400 MHz, DMSO-d₆) δ 6.89-6.82 (m, 2H), 6.58-6.53 (m, 2H), 5.27 (t, J=5.6 Hz, 1H), 5.14 (s, 2H), 3.00 (s, 3H), 2.99-2.92 (m, 2H), 0.91 (t, J=7.2 Hz, 3H).

Compound 66B was subjected to treatment with intermediate 12F using conditions as described for compound 7 to yield the intermediate 66C. HCl/EtOAc (4M, 1.11 mL) was added to compound 66C (100 mg, 263 umol) in EA (5 mL). The mixture was stirred at 20° C. for 1 h. The mixture was concentrated under vacuum. The product was purified by preparatory-HPLC (water (0.05% HCl)-ACN). Compound 66 (E-isomer, 30.0 mg, yield 36.0%) was obtained as a yellow solid. ¹H NMR (400 MHz, CD₃OD) δ 7.41-7.34 (m, 4H), 7.19 (d, J=80.0 Hz, 1H), 4.13 (d, J=2.8 Hz, 2H), 3.87 (d, J=2.0 Hz, 2H), 3.24 (s, 3H), 3.22-3.15 (m, 2H), 1.09 (t, J=7.2 Hz, 3H). ¹⁹F NMR (376 MHz, CD₃OD) δ−115.16. MS (ESI) m/z (M+H)⁺ 281.2.

Compound 67 (Z-isomer, 20.0 mg, yield 24.0%) was obtained as a yellow solid. ¹H NMR (400 MHz, CD₃OD) δ 7.34-7.28 (m, 4H), 7.28-7.08 (m, 1H), 4.20 (d, J=2.4 Hz, 2H), 3.72 (d, J=2.4 Hz, 2H), 3.22 (s, 3H), 3.20-3.15 (m, 2H), 1.08 (t, J=7.2 Hz, 3H). ¹⁹F NMR (376 MHz, CD₃OD) δ−117.25. MS (ESI) m/z (M+H)⁺ 281.2.

### (E)-2-(aminomethyl)-3-fluoroalkyl phenylcarbamate (65) and

### (Z)-2-(aminomethyl)-3-fluoroalkyl phenylcarbamate (68)

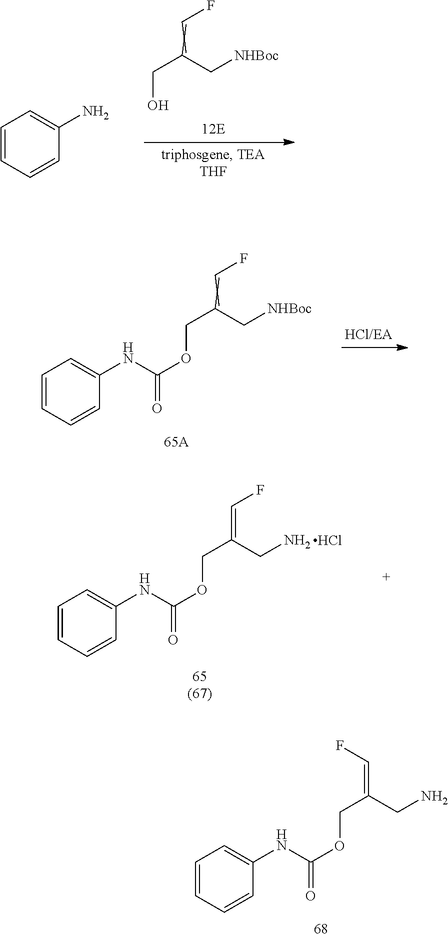

To a solution of Triphosgene (1.01 g, 3.40 mmol) in THF (10 mL) was added a solution of aniline (250 mg, 2.68 mmol) in THF (2 mL). Then TEA (0.8 mL, 5.64 mmol) was added dropwise. The mixture was stirred at 25° C. for 2 h. Then the solvent was removed in vacuo. The residue was dissolved in CH₃CN (10 mL). Intermediate 12E (550.93 mg, 2.68 mmol) and TEA (0.8 mL 5.64 mmol) was added. The mixture was stirred at 70° C. for 12 h. The solid was filtered off. The filtrate was collected and concentrated in vacuo. The residue was purified by column (PE:EA=1:0 2:1) to give compound 65A (550 mg, yield 61.3%) as light yellow oil. MS (ESI) m/z (M+Na)⁺ 346.9.

Compound 65A (540 mg, 1.66 mmol) in HCl/EtOAc (4M, 10 mL) was stirred at 25° C. for 1 h. The solvent was removed in vacuo. The residue was dissolved in H₂O (2 mL) and CH₃CN (2 mL). The solution was purified by preparatory-HPLC (HCl). Compound 65 (190 mg, yield 43.7%) was obtained as white solid and compound 68 (35 mg, yield: 7.84%) was obtained as white solid. Compound 65: ¹H NMR (DMSO-d₆, 400 MHz) δ 9.94 (br. s, 1H), 8.31 (br. s, 3H), 7.48 (d, J=8.0 Hz, 2H), 7.40 (s, 0.5H), 7.33-7.24 (m, 2H), 7.20 (s, 0.5H), 7.04-6.96 (m, 1H), 4.70-4.60 (m, 2H), 3.63-3.51 (m, 2H). MS (ESI) m/z (M+H)⁺ 225.0.

Compound 68: ¹H NMR (DMSO-d₆, 400 MHz) δ 9.89 (br. s, 1H), 8.19 (br. s, 3H), 7.43 (d, J=8.0 Hz, 2H), 7.31-7.20 (m, 2.6H), 7.04 (s, 0.5H), 6.99-6.92 (m, 1H), 4.79-4.68 (m, 2H), 3.54-3.44 (m, 2H). MS (ESI) m/z (M+H)⁺ 225.0.

### (E)-2-(aminomethyl)-3-fluoroalkyl phenylcarbamate (71)

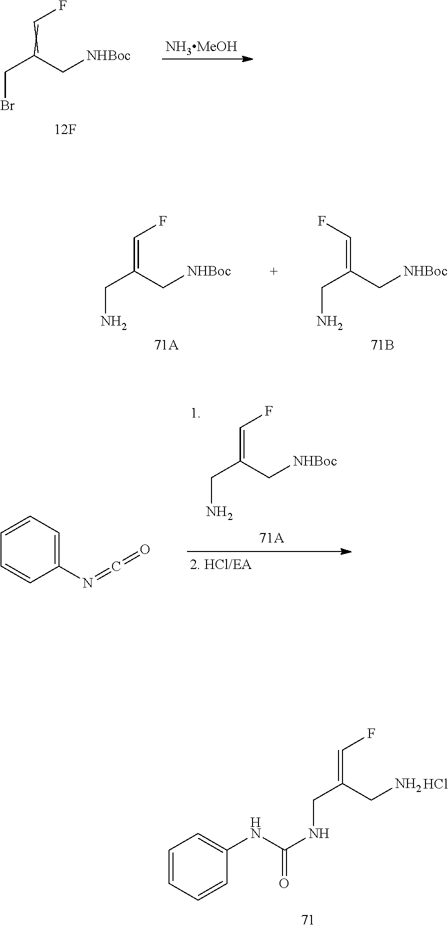

Intermediate 12F (500 mg, 1.86 mmol) was added to NH₃/MeOH (0.7M, 2.7 mL). The mixture was stirred at 20° C. for 12 h. The reaction mixture was concentrated under reduced pressure to remove solvent. The residue was purified by preparatory-HPLC (water (10 mM NH₄HCO₃)-ACN) to afford two isomers. Intermediate 71A (Z-isomer, 30.0 mg, yield 7.88%) was obtained as a yellow oil. ¹H NMR (400 MHz, CDCl₃) δ 6.72-6.38 (m, 1H), 4.98 (br. s., 1H), 3.95 (d, J=4.4 Hz, 2H), 3.20 (br. s., 2H), 1.44 (s, 9H), 1.24 (s, 2H). Intermediate 71B (E-isomer, 10.0 mg, yield 2.63%) was obtained as a yellow oil. ¹H NMR (400 MHz, CDCl₃) δ 6.71-6.37 (m, 1H), 5.01 (br. s., 1H), 3.72 (br. s., 2H), 3.42 (d, J=2.0 Hz, 2H), 1.44 (s, 9H), 1.25 (s, 2H).

Phenyl isocyanate solution in DCM (7.5 mL) was stirred at 20° C. for 0.5 h with intermediate 71A (30.0 mg, 0.147 mmol). The reaction was poured into ice-water (10 mL) and then extracted with DCM (10 mL×3). The combined organic layer was washed with brine (30 mL), dried over anhydrous Na₂SO₄, filtered and then concentrated. The residue was purified by column chromatography (SiO₂, 34% EtOAc in PE). The resulting intermediate was taken up in EtOAc (4 mL) was treated with HCl/EtOAc (6 mL). It was stirred at 20° C. for 2 h. The reaction mixture was concentrated under reduced pressure to afford compound 71 (27 mg, HCl) as yellow oil. ¹H NMR (400 MHz, CD₃OD) δ 7.43-7.34 (m, 2H), 7.28-7.24 (m, 2H), 7.09-6.83 (m, 2H), 3.87 (d, J=2.4 Hz, 2H), 3.70 (s, 2H). ¹⁹F NMR (400 MHz, CD₃OD) δ−127.61. MS (ESI) m/z (M+H)⁺ 224.1.

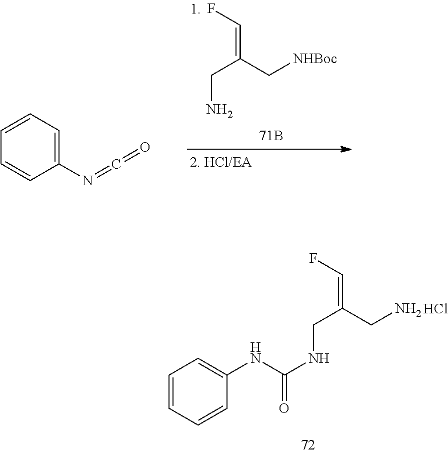 [SMILES: CC(C)(C)OC(=O)NC/C(=C/F)CN ; O=C=Nc1ccccc1 ; NC/C(=C/F)CNC(=O)Nc1ccccc1 ; Cl]

Phenyl isocyanate solution in DCM (7.5 mL) was treated with intermediate 71B using conditions as described for compound 71. Compound 72 (9.6 mg, HCl) was obtained as a yellow solid. ¹H NMR (400 MHz, CD₃OD) δ 7.44-7.35 (m, 2H), 7.26 (t, J=7.2 Hz, 2H), 7.11-6.98 (m, 2H), 4.04 (d, J=1.6 Hz, 2H), 3.53 (s, 2H). ¹⁹F NMR (400 MHz, CD₃OD) δ−125.07. MS (ESI) m/z (M+H)⁺ 224.1.

### Example 21

### Compounds 76-78

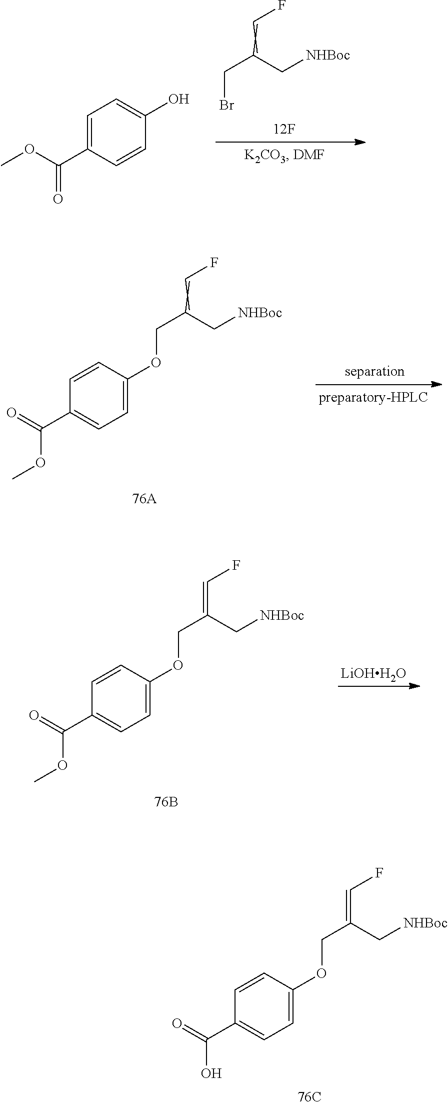 [SMILES: COC(=O)c1ccc(O)cc1 ; CC(C)(C)OC(=O)NCC(=CF)CBr ; COC(=O)c1ccc(OCC(=CF)CNC(=O)OC(C)(C)C)cc1 ; COC(=O)c1ccc(OC/C(=C/F)CNC(=O)OC(C)(C)C)cc1 ; "LiOH·H2O" ; CC(C)(C)OC(=O)NC/C(=C\F)COc1ccc(C(=O)O)cc1]

K₂CO₃ (387 mg, 2.80 mmol) was added to the mixture of compound intermediate 12F (500 mg, 1.86 mmol) and methyl 4-hydroxybenzoate (289 mg, 1.90 mmol) in DMF (10 mL) at 20° C. for 3 h. The mixture was added H₂O (100 mL) and extracted with EA (50 mL×2), the organic phase was washed with 1M NaOH (40 mL), brine (40 mL) and dried over Na₂SO₄, filtered and concentrated under vacuum. The crude product 76A was for purified by preparatory-SFC (0.1% NH₃H₂O EtOH) (RT: 2.539 min). Compound 76B (377.4 mg, yield 59.64%) was obtained as a white solid. ¹H NMR (DMSO-d₆, 400 MHz): δ 7.91 (d, J=8.8 Hz, 2H), 7.20-6.94 (m, 4H), 4.51 (d, J=3.3 Hz, 2H), 3.81 (s, 3H), 3.76 (br d, J=4.5 Hz, 2H), 1.33 (s, 9H).

To a solution of methyl ester intermediate 76B (377.4 mg, 1.11 mmol) in THF (5 mL) and H₂O (5 mL) was added LiOH.H₂O (467 mg, 11.12 mmol). The mixture was stirred at 25° C. for 6 h. The mixture was adjusted to pH 4 with 1N HCl, extracted with EtOAc (20 mL×2). The organics were collected, washed with brine (30 mL), dried with Na₂SO₄, filtered and concentrated to give compound 76C (360 mg, crude) as white solid, which was used directly for the next step without further purification.

### (E)-4-((2-(aminomethyl)-3-fluoroalkyl)oxy)-N-(5-(5-(2-oxohexahydro-1H-thieno[3,4-d]imidazol-4-yl)pentanamido)pentyl)benzamide (76)

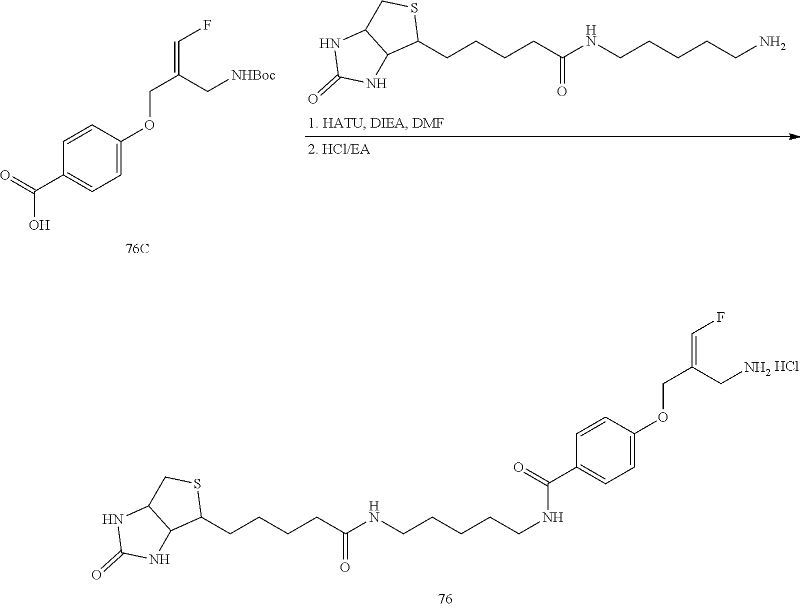 [SMILES: CC(C)(C)OC(=O)NC/C(=C\F)COc1ccc(C(=O)O)cc1 ; NCCCCCNC(=O)CCCCC1SCC2NC(=O)NC21 ; NC/C(=C\F)COc1ccc(C(=O)NCCCCCNC(=O)CCCCC2SCC3NC(=O)NC32)cc1 ; Cl]

To a solution of compound 76C (70 mg, 215.17 umol) and N-(5-aminopentyl)-5-(2-oxohexahydro-1H-thieno[3,4-d]imidazol-4-yl)pentanamide (85 mg, 258.20 umol) in DMF (4 mL) was added HATU (90 mg, 236.68 umol), DIEA (0.09 mL, 537.91 umol). The mixture was stirred at 25° C. for 12 h. The solvent was removed in vacuo. The residue was purified by preparatory-HPLC (basic) to give compound intermediate 76D (95 mg, yield: 69.44%) as white solid. ¹H NMR (DMSO-d₆, 400 MHz): δ 8.31-8.18 (m, 1H), 7.76 (d, J=8.4 Hz, 2H), 7.73-7.67 (m, 1H), 7.12 (br. s, 0.5H), 7.09-7.01 (m, 1H), 6.99-6.87 (m, 2.5H), 6.43-6.27 (m, 2H), 4.49-4.40 (m, 2H), 4.30-4.22 (m, 1H), 4.12-4.05 (m, 1H), 3.76-3.66 (m, 2H), 3.22-3.13 (m, 2H), 3.10-3.02 (m, 1H), 3.01-2.94 (m, 2H), 2.82-2.73 (m, 1H), 2.57-2.50 (m, 1H), 2.04-1.95 (m, 2H), 1.65-1.17 (m, 21H). To a solution of compound 76D (90 mg, 141.56 umol) in EtOAc (2 mL) was added HCl/EtOAc (4M, 5 mL). The mixture was stirred at 25° C. for 30 min. The solvent was removed in vacuo. The residue was dissolved in H₂O (3 mL) and freeze dried in vacuo to give compound 76 (70 mg, yield: 86.43%) as yellow solid. ¹H NMR (DMSO-d₆, 400 MHz): δ 8.48-8.22 (m, 4H), 7.87-7.75 (m, 3H), 7.42 (br. s, 0.5H), 7.21 (br. s, 0.5H), 7.07-7.00 (m, 2H), 4.72-4.67 (m, 2H), 4.33-4.26 (m, 1H), 4.14-4.08 (m, 1H), 3.63-3.54 (m, 2H), 3.26-3.16 (m, 2H), 3.12-3.05 (m, 1H), 3.04-2.97 (m, 2H), 2.86-2.77 (m, 1H), 2.60-2.54 (m, 1H), 2.08-2.00 (m, 2H), 1.66-1.54 (m, 1H), 1.53-1.36 (m, 7H), 1.33-1.21 (m, 4H).

### (E)-4-((2-(aminomethyl)-3-fluoroalkyl)oxy)-N-(13-oxo-17-(2-oxohexahydro-1H-thieno[3,4-d]imidazol-4-yl)-3,6,9-trioxa-12-azaheptadecyl)benzamide (77)

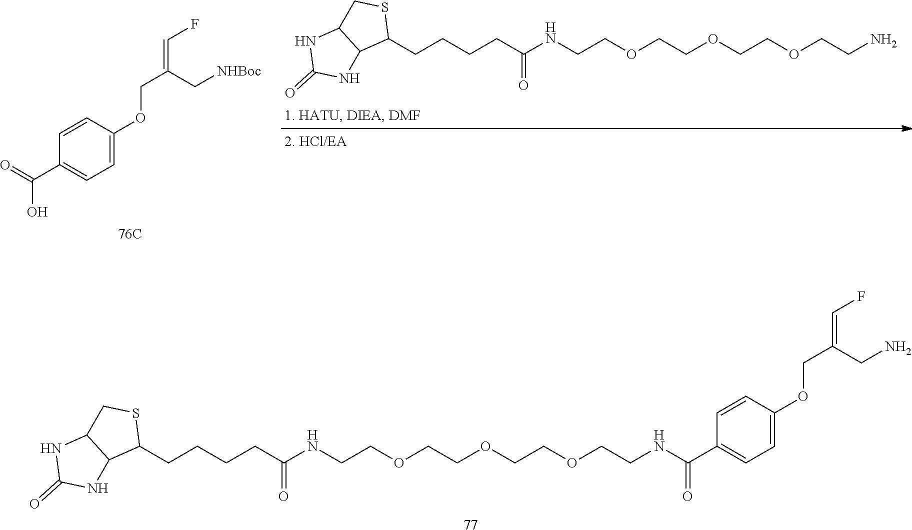 [SMILES: CC(C)(C)OC(=O)NC/C(=C\F)COc1ccc(C(=O)O)cc1 ; NCCOCCOCCOCCNC(=O)CCCCC1SCC2NC(=O)NC21 ; NC/C(=C\F)COc1ccc(C(=O)NCCOCCOCCOCCNC(=O)CCCCC2SCC3NC(=O)NC32)cc1 ; "77"]

Intermediate 76C and N-(2-(2-(2-(2-aminoethoxy)ethoxy)ethoxy)ethyl)-5-(2-oxohexahydro-1H-thieno[3,4-d]imidazol-4-yl)pentanamide were subjected to same conditions as described for compound 76 to obtain compound 77. Compound 77 (50 mg, yield: 84.32%) was obtained as colorless oil. ¹H NMR (DMSO-d₆, 400 MHz): δ 8.44-8.38 (m, 1H), 8.34 (br. s, 3H), 7.88-7.76 (m, 3H), 7.39 (br. s, 0.5H), 7.18 (br. s, 0.5H), 7.05-6.97 (m, 2H), 4.70-4.64 (m, 2H), 4.30-4.24 (m, 1H), 4.10-4.08 (m, 1H), 3.59-3.52 (m, 2H), 3.51-3.41 (m, 10H), 3.39-3.31 (m, 4H), 3.17-3.10 (m, 2H), 3.09-3.02 (m, 1H), 2.82-2.74 (m, 1H), 2.58-2.51 (m, 1H), 2.06-1.98 (m, 2H), 1.62-1.52 (m, 1H), 1.50-1.36 (m, 3H), 1.33-1.17 (m, 2H).

### Compound 78

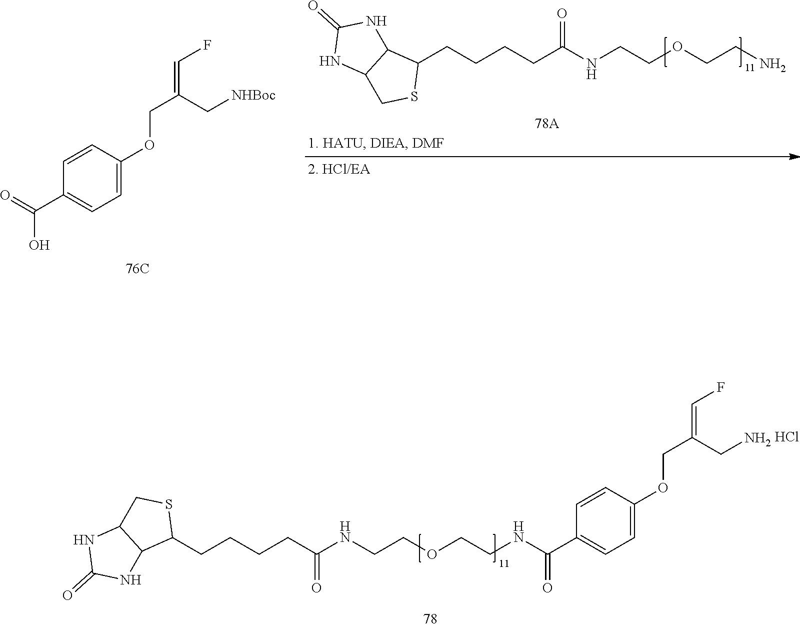

Intermediate 76C and compound 78A were subjected to same conditions as described for compound 76 to obtain compound 78. Compound 78 (75 mg, yield: 87.70%) was obtained as colorless oil. ¹H NMR (DMSO-d₆, 400 MHz): δ 8.42-8.33 (m, 1H), 8.23 (br. s, 3H), 7.86-7.76 (m, 3H), 7.40 (s, 0.5H), 7.19 (s, 0.5H), 7.04-6.98 (m, 2H), 4.69-4.61 (m, 2H), 4.31-4.23 (m, 1H), 4.13-4.05 (m, 1H), 3.52-3.41 (m, 44H), 3.39-3.32 (m, 4H), 3.19-3.11 (m, 2H), 3.09-3.01 (m, 1H), 2.82-2.74 (m, 1H), 2.57-2.51 (m, 1H), 2.07-1.98 (m, 2H), 1.63-1.51 (m, 1H), 1.51-1.36 (m, 3H), 1.32-1.18 (m, 2H).

### Example 22

### Compounds 79-81

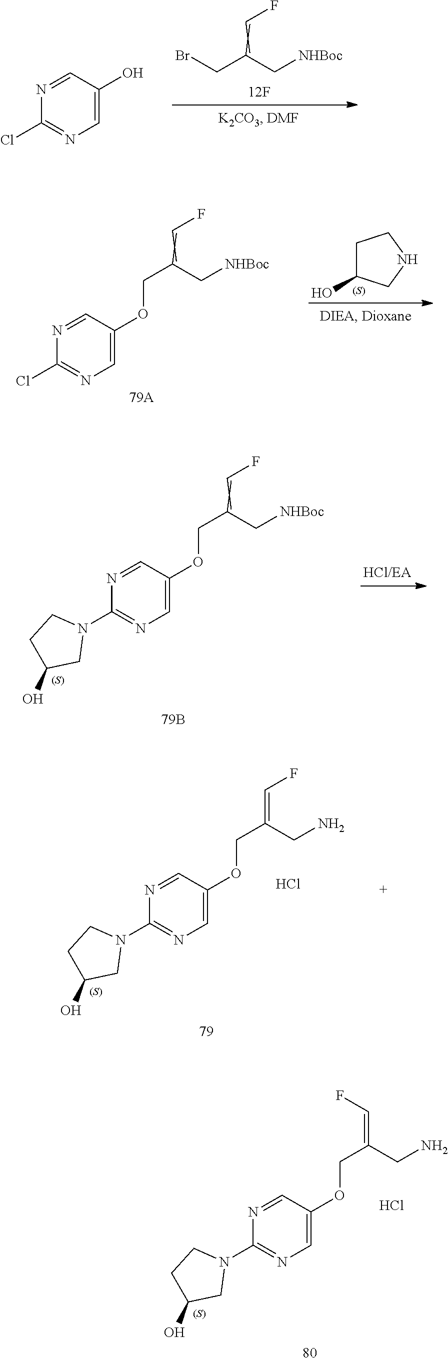

K₂CO₃ (387 mg, 2.80 mmol) was added to the mixture of intermediate 12F (500 mg, 1.86 mmol) and 2-chloropyrimidin-5-ol (243 mg, 1.86 mmol) in DMF (8 mL) and it was stirred at 20° C. for 3 h. The mixture was added H₂O (80 mL) and extracted with EA (50 mL), the organic phase was washed with 1M NaOH (30 mL), brine (30 mL) and dried over Na₂SO₄, filtered and concentrated under vacuous. Compound 79A (500 mg, yield 82.1%) was obtained as a yellow oil, which was used for next step without purification.

Compounds 79A (250 mg, 0.79 mmol) and (3S)-pyrrolidin-3-ol (206 mg, 2.36 mmol) were dissolved in dioxane (7 mL) and DIEA (1.2 mL, 7.08 mmol). Flushed the solution with N2 gas, seal the vessel, and heat the mixture to 120° C. for 1.5 hours by microwave. The mixture was concentrated under vacuum to afford compound 79B (405 mg, crude) as a brown oil, which was used for next step without purification.

HCl/EtOAc (4M, 0.5 mL) was added to the mixture of compound 79B (80 mg, 217.15 umol) in EA (5 mL) at 20° C. for 1 h. The mixture was concentrated under vacuo and the product was purified by preparatory-HPLC. Compound 79 (28.5 mg, HCl) was obtained as a yellow solid and compound 80 (3.8 mg, HCl) was obtained as yellow oil.

Compound 79 (E isomer): ¹H NMR (400 MHz, CD₃OD) δ 8.18 (s, 2H), 7.09 (d, J=82.0 Hz, 1H), 4.57 (d, J=3.6 Hz, 2H), 4.53-4.47 (m, 1H), 3.71 (d, J=1.6 Hz, 2H), 3.66-3.58 (m, 3H), 3.56-3.49 (m, 1H), 2.18-2.07 (m, 1H), 2.06-1.97 (m, 1H). ¹⁹F NMR (400 MHz, CD₃OD) δ−125.26. MS (ESI) m/z (M+H)⁺ 269.1.

Compound 80 (Z isomer): ¹H NMR (400 MHz, CD₃OD) δ 8.50 (s, 2H), 7.19 (d, J=80.4 Hz, 1H), 4.90 (d, J=2.4 Hz, 2H), 4.62 (s, 1H), 3.88-3.64 (m, 6H), 2.32-2.08 (m, 2H). ¹⁹F NMR (400 MHz, CD₃OD) δ−119.92. MS (ESI) m/z (M+H)⁺ 269.1.

### Compound 81

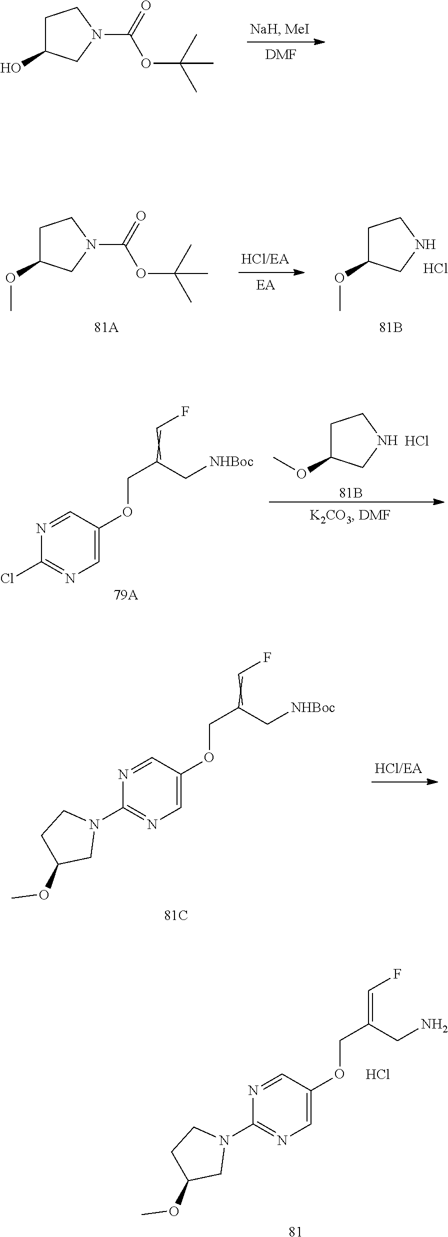

To a solution of tert-butyl (S)-3-hydroxypyrrolidine-1-carboxylate (1 g, 5.34 mmol) in DMF (10 mL) was added NaH (256.3 mg, 6.41 mmol, 60% purity). The mixture was stirred for 30 min at 0° C. Then Mel (24.66 mmol, 1.54 mL) was added. The mixture was stirred at 25° C. for 12 h. The mixture was quenched with H₂O (100 mL), extracted with EA (100 mL), washed with brine (100 mL×3), dried over Na₂SO₄ and concentrated. The residue was purified by column chromatography (SiO₂, Petroleum ether/Ethyl acetate=20:1 to 1:1). Compound 81A (1.1 g, crude) was obtained as a colorless oil. ¹H NMR (400 MHz, DMSO-d₆) δ 3.90 (br s, 1H), 3.31-3.14 (m, 6H), 1.93-1.82 (m, 2H), 1.40 (s, 9H)

To a solution of compound 81A (1.1 g, 5.47 mmol) in EA (15 mL) was added HCl/EtOAc (4M, 14 mL). The mixture was stirred at 25° C. for 1 h. The mixture was concentrated. Compound 81B (700.0 mg, crude, HCl) was obtained as a colorless oil. The crude product was used in next step directly.

To a solution of compound 79A (500.0 mg, 1.57 mmol) in DMF (20 mL) was added K₂CO₃ (652.4 mg, 4.72 mmol) and compound 81B (281.5 mg, 2.05 mmol, HCl). The mixture was stirred at 100° C. for 2 h. The mixture was diluted with H₂O (50 mL), extracted with EA (50 mL), washed with brine, dried over Na₂SO₄ and concentrated. The residue was purified by column chromatography (SiO₂, Petroleum ether/Ethyl acetate=10/1 to 1/1). Compound 81C (300.0 mg, 47.6% yield, 95.4% purity) was obtained as a yellow oil. ¹H NMR (400 MHz, CDCl₃) δ 8.13-8.09 (m, 2H), 6.68 (d, J=82.0 Hz, 1H), 4.78 (br.s., 1H), 4.39-4.35 (m, 2H), 4.09-4.04 (m, 1H), 4.01-3.97 (m, 2H), 3.78-3.55 (m, 4H), 3.36 (s, 3H), 2.20-2.05 (m, 2H), 1.43 (s, 9H). MS (ESI) m/z (M+H)⁺ 383.2.

To a solution of compound 81C (300.0 mg, 784.46 umol) in EA (10 mL) was added HCl/EtOAc (4M, 4 mL). The mixture was stirred at 25° C. for 1 h. The mixture was concentrated. The residue was purified by preparatory-HPLC (HCl condition). Compound 81 (140.0 mg, 55.43% yield, 99.0% purity, HCl) was obtained as a yellow solid. ¹H NMR (400 MHz, DMSO-d₆) δ 8.43-8.32 (m, 3H), 8.31 (s, 2H), 7.38-7.09 (m, 1H), 4.68-4.60 (m, 2H), 4.07-4.00 (m, 1H), 3.62-3.51 (m, 5H), 3.49-3.37 (m, 1H), 3.23 (s, 3H), 2.08-1.99 (m, 2H). MS (ESI) m/z (M+H)⁺ 283.1.

### Biological Data

### Example 23

VAP-1, DAO, MAO-A, MAO-B, and HRP activity and inhibition thereof was assessed by means of a continuous fluorescence assay. The Amplex Red Hydrogen Peroxide/Peroxidase Assay (ThermoFisher) was optimized for detecting amine oxidase (AO) activity. In an enzyme-coupled reaction, the H₂O₂ generated by AO reacts 1:1 with Amplex Red reagent to produce fluorescent product resorufin.

Assays were typically setup in black 384-well plates using automated liquid handling as follows. VAP-1/MAO assay buffer contains 50 nM sodium phosphate, pH 7.4; DAO assay buffer contains 50 mM HEPES, pH 7.5; HRP assay buffer contains 100 mM CHES, 1 mM MgCl₂, 50 mM NaCl, 0.05% BSA, pH 9.0. Inhibitors were serially diluted in DMSO and used to setup 2× mixtures with AO in the aforementioned buffer. After 30 min pre-incubation at 37° C. (VAP-1, HRP) or room temp (DAO, MAO), the reaction was initiated by adding a 2× mix of Amplex Red, HRP (coupling enzyme), and substrate (benzylamine, histamine, tyramine, benzylamine, H₂O₂, for VAP-1, DAO, MAO-A, MAO-B, and HR, respectively) in the same buffer. Reaction progress curve data were typically collected for 5 min using excitation/emission wavelengths of 510 nm/565 nm on FLIPR-Tetra plate readers (Molecular Devices Inc). Reaction rates were calculated from progress curve slopes typically over 60-150 sec. Dose response curves (rate vs. log inhibitor concentration) were typically fit to a 4-parameter logistic function to extract IC50 values.

### VAP-1 Inhibition

TABLE 2
VAP-1 inhibition assay
Column A: Human VAP-1 IC50
Column B: Human MAO-A IC50
Column C: Human MAO-B IC50
Column D: Human DAO IC50

| Compound | Column | Column | Column | Column |
|---|---|---|---|---|
| No. | A | B | C | D |
| 1 | C | C | C | ND |
| 2 | B | C | C | ND |
| 3 | A | C | A | C |
| 4 | A | C | C | A |
| 5 | A | A | A | C |
| 6 | A | A | A | B |
| 7 | A | B | A | B |
| 8 | A | A | A | A |
| 9 | A | B | A | C |
| 10 | A | C | A | A |
| 11 | A | A | A | A |
| 12 | C | C | C | ND |
| 13 | A | A | A | B |
| 14 | A | A | A | B |
| 15 | A | A | A | C |
| 16 | A | A | A | A |
| 17 | B | C | C | ND |
| 18 | A | A | A | B |
| 19 | A | B | A | A |
| 20 | A | A | A | A |
| 21 | A | B | C | C |
| 22 | C | C | B | ND |
| 23 | A | C | B | C |
| 24 | A | B | A | C |
| 25 | A | B | A | A |
| 26 | A | B | A | C |
| 27 | A | C | A | ND |
| 28 | A | C | A | A |
| 29 | A | A | A | A |
| 30 | A | A | A | A |
| 31 | C | C | C | ND |
| 32 | C | C | C | ND |
| 33 | A | A | A | C |
| 34 | A | A | A | A |
| 37 | A | B | A | A |
| 38 | A | A | A | A |
| 39 | A | C | A | A |
| 40 | A | B | A | A |
| 41 | A | A | A | C |
| 42 | A | A | A | A |
| 43 | A | B | A | A |
| 44 | A | A | A | A |
| 45 | A | C | A | C |
| 46 | A | C | A | B |
| 47 | A | A | C | C |
| 48 | A | C | A | A |
| 49 | A | A | A | A |
| 50 | A | B | A | A |
| 51 | A | C | C | B |
| 52 | A | C | C | A |
| 53 | A | B | A | A |
| 54 | A | B | A | A |
| 55 | A | A | A | C |
| 56 | A | A | A | A |
| 57 | A | A | B | A |
| 58 | A | A | A | A |
| 59 | A | C | A | C |
| 60 | A | A | A | A |
| 61 | A | C | C | A |
| 62 | A | C | C | A |
| 63 | A | C | C | A |
| 64 | A | B | A | A |
| 65 | A | B | B | A |
| 66 | A | C | C | C |
| 67 | A | C | B | B |
| 68 | A | A | A | A |
| 69 | A | C | A | C |
| 70 | A | A | C | C |
| 71 | A | A | C | B |
| 72 | A | A | C | A |
| 76 | ND | C | A | A |
| 77 | ND | C | B | A |
| 78 | A | C | C | A |
| 79 | A | C | B | A |
| 80 | A | C | C | A |
| 81 | A | C | C | A |
| 82 | A | B | A | B |

A: <3 uM;
B: 3-10 uM;
C: >10 uM;
ND: Not Determined

### Carbon Tetrachloride-Induced Liver Fibrosis in Mice or Rats

Carbon tetrachloride-induced liver fibrosis is a widely used and accepted model for evaluating novel antifibrotic therapies. The methods for inducing liver fibrosis by carbon tetrachloride administration is described in Lee, J Clin Invest, 1995 and Tsukamoto, Semin Liver Dis, 1990. Briefly, male C57BL/6 mice are challenged with 1 mg/kg carbon tetrachloride (Sigma Aldrich, diluted 1:7 in corn or olive oil) administered by intraperitoneal injection twice weekly for a period of 4 weeks. Mice are euthanized on day 28. In an alternative implementation, Wistar rats are administered carbon tetrachloride by intraperitoneal injection three times per week for 8-12 weeks. Rats are euthanized at the termination of the experiment, 8-12 after study initiation.

Blood is collected by cardiac puncture and processed into serum for evaluation of liver enzymes (including ALT, AST, ALP, etc) at several timepoints throughout the study and at termination of the study. The liver tissues from all animals are collected and fixed by immersion in 10% neutral buffered formalin, processed, paraffin embedded, sectioned, mounted, and stained with Masson's Trichrome (Tri) or Picrosirius Red (PSR) using standard histological methods for evaluation of fibrosis severity.

### Mouse Unilateral Ureteral Obstruction Kidney Fibrosis Model

Female C57BL/6 mice (Harlan, 4-6 weeks of age) will be given free access to food and water and allowed to acclimate for at least 7 days prior to test initiation. After acclimation, mice are anesthetized and undergo unilateral ureteral obstruction (UUO) surgery or sham to left kidney. Briefly, a longitudinal, upper left incision is performed to expose the left kidney. The renal artery is located and 6/0 silk thread is passed between the artery and the ureter. The thread is looped around the ureter and knotted 3 times insuring full ligation of ureter. The kidney is returned to abdomen, the abdominal muscle is sutured and the skin is stapled closed. All animals are euthanized 4, 8, 14, 21, or 28 days after UUO surgery. Following sacrifice blood is collected via cardiac puncture, the kidneys are harvested and one half of the kidney is frozen at −80° C. and the other half is fixed in 10% neutral buffered formalin for histopathological assessment of kidney fibrosis.

### Bleomycin Dermal Fibrosis Model

Bleomycin (Calbiochem, Billerica Mass.) is dissolved in phosphate buffered saline (PBS) at 10 ug/ml, and sterilized by filtration. Bleomycin or PBS control is injected subcutaneously into two locations on the shaved back of C57/BL6 or S129 mice (Charles River/Harlan Labs, 20-25 g) once daily for 28 days while under isoflourane anesthesia (5% in 100% 02). After 28 days, mice are euthanized and 6 mm-full thickness punch biopsies are obtained from each injection site. Dermal fibrosis is assessed by standard histopathology and hydroxyproline biochemical assays.

### Example 24: Targeting VAP-1

### Inhibition of EpMT

For assessment of in vitro EMT, NMuMG cells (ATCC) are grown to confluence in 10% serum (Fetal Bovine Serum) growth media (Dubecco's Modified Eagles Medium supplemented with 10 ug/mL insulin) and then are followed by 24 h starvation in 0.5% serum media+/− drug inhibitors. Cells are then treated with recombinant human TGFb1 (R&D Systems 5 ng/mL)⁺/− drug inhibitors in 0.5% serum media. For time points greater than 24 h, the aforementioned media is refreshed every 24 hours. Cell lysates were analyzed for aSMA protein expression by western blot.
Miettinen et al. (1994). “TGF-beta induced transdifferentiation of mammary epithelial cells to mesenchymal cells: involvement of type I receptors.” J Cell Biol 127 (6 Pt 2):2021-36.
Lamouille et al. (2014). “Molecular mechanisms of epithelial-mesenchymal transition.” Nat Rev Mol Cell Biol 15(3):178-96.

For assessment of in vitro FMT, Normal Human Lung Fibroblasts (NHLF) cells (Lonza) were grown in Fibroblast Growth Media-2 (Lonza CC-3131/with CC-4126 bullet kit) and then were followed by 24 h starvation in serum/growth factor free Fibroblast Basal Media-2 (Lonza CC-3131)+/− drug inhibitors. Cells were then treated with TGFb1 (5 ng/mL) Fibroblast Basal Media+/− drug inhibitors. Cell lysates are analyzed for aSMA protein expression by western blot.

Further details may be found in Pegorier et al. (2010). “Bone Morphogenetic Protein (BMP)-4 and BMP-7 regulate differentially Transforming Growth Factor (TGF)-B1 in normal human lung fibroblasts (NHLF)” Respir Res 11:85, which is incorporated herein by reference in its entirety.

### Example 25: Human Treatment

The efficacy of treatment with a compound of a preferred embodiment compared with placebo in patients with idiopathic pulmonary fibrosis (IPF) and the safety of treatment with a compound of a preferred embodiment compared with placebo in patients with IPF is assessed. The primary outcome variable is the absolute change in percent predicted forced vital capacity (FVC) from baseline to Week 52. Other possible end-points would include, but are not limited to: mortality, progression free survival, change in rate of FVC decline, change in Sp02, and change in biomarkers (HRCT image analysis; molecular and cellular markers of disease activity). Secondary outcome measures include: composite outcomes of important IPF-related events; progression-free survival; the rate of death from any cause; the rate of death from IPF; categorical assessment of absolute change in percent predicted FVC from baseline to Week 52; change in Shortness-of-Breath from baseline to Week 52; change in percent predicted hemoglobin (Hb)-corrected carbon monoxide diffusing capacity (DLco) of the lungs from baseline to Week 52; change in oxygen saturation during the 6 minute walk test (6MWT) from baseline to Week 52; change in high-resolution computed tomography (HRCT) assessment from baseline to Week 52; change in distance walked in the 6MWT from baseline to Week 52. Patients eligible for this study include, but are not limited to: those patients that satisfy the following inclusion criteria: diagnosis of IPF; 40 to 80 years of age; FVC≥50% predicted value; DLco≥35% predicted value; either FVC or DLco≤90% predicted value; no improvement in past year; a ratio of the forced expiratory volume in 1 second (FEV1) to the FVC of 0.80 or more; able to walk 150 meters in 6 minutes and maintain saturation ≥83% while on no more than 6 L/min supplemental oxygen. Patients are excluded from this study if they satisfy any of the following criteria: unable to undergo pulmonary function testing; evidence of significant obstructive lung disease or airway hyper-responsiveness; in the clinical opinion of the investigator, the patient is expected to need and be eligible for a lung transplant within 52 weeks of randomization; active infection; liver disease; cancer or other medical condition likely to result in death within 2 years; diabetes; pregnancy or lactation; substance abuse; personal or family history of long QT syndrome; other IPF treatment; unable to take study medication; withdrawal from other IPF trials. Patients are orally dosed with either placebo or an amount of a compound of a preferred embodiment (1 mg/day-1000 mg/day). The primary outcome variable will be the absolute change in percent predicted FVC from Baseline to Week 52. Patients will receive blinded study treatment from the time of randomization until the last patient randomized has been treated for 52 weeks. Physical and clinical laboratory assessments will be performed at defined intervals during the treatment duration, for example at weeks 2, 4, 8, 13, 26, 39, and 52. Pulmonary function, exercise tolerance, and shortness-of-breath will be assessed at defined intervals during the treatment duration, for example at weeks 13, 26, 39, and 52. A Data Monitoring Committee (DMC) will periodically review safety and efficacy data to ensure patient safety.

### Example Trial in SSc

The efficacy of treatment with a compound of a preferred embodiment compared with placebo in patients with systemic sclerosis (SSc) and the safety of treatment with a compound of a preferred embodiment compared with placebo in patients with SSc is assessed. The primary outcome variable is the absolute change in Modified Rodnan Skin Score (mRSS) from baseline to Week 48. Other possible end-points would include, but are not limited to: mortality, percentage of patients with treatment-emergent adverse events (AEs) and serious adverse events (SAEs), composite measurement of disease progression, and change in biomarkers (molecular and cellular markers of disease activity, such as C-reactive protein). Secondary outcome measures include, but are not limited to: Scleroderma Health Assessment Questionnaire (SHAQ) score; the Health Assessment Questionnaire Disability Index (HAQ-DI); Functional Assessment of Chronic Illness Therapy-Fatigue (FACIT) score; severity of pruritus as measured by a standardized scale, such as the 5-D Itch Scale; St. George's Respiratory Questionnaire (SGRQ) score; Tender Joint Count 28 (TCJ28); lung function parameters; standard vital signs (including blood pressure, heart rate, and temperature); electrocardiogram measurements (ECGs); laboratory tests (clinical chemistry, hematology, and urinalysis); pharmacokinetics (PK) measurements. Included in these measurements and in addition, clinical and biomarker samples, such as skin biopsies and blood (or serum and/or plasma), will also be collected prior to initiation of treatment. Additionally, patients eligible for this study include, but are not limited to, those patients that satisfy the following criteria: Patients at least 18 years of age; diagnosis of SSc according to the American College of Rheumatology (ACR) and European League Against Rheumatism (EULAR) Criteria, meeting criteria for active disease and with a total disease duration of less than or equal to 60 months; 10≤mRSS≤35. Patients are excluded from this study if they satisfy any of the following criteria: major surgery within 8 weeks prior to screening; scleroderma limited to area distal to the elbows or knees; rheumatic autoimmune disease other than SSc; use of any investigational, biologic, or immunosuppressive therapies, including intra-articular or parenteral corticosteroids within 4 weeks of screening. Patients are orally dosed with either placebo or an amount of a compound of a preferred embodiment (1 mg/day-1000 mg/day). The primary outcome variable will be the absolute change in mRSS from Baseline to Week 48. Patients will receive blinded study treatment from the time of randomization until the last patient randomized has been treated for 48 weeks. Physical and clinical laboratory assessments will be performed at defined intervals during the treatment duration, such as Weeks 2, 4, 8, 12, 24, 36, and 48. Clinical and biomarker samples will also be collected at Week 48. A Data Monitoring Committee (DMC) will periodically review safety and efficacy data to ensure patient safety.

While some embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the present technology. This includes the generic description of the present technology with a proviso or negative limitation removing any subject matter from the genus, regardless of whether or not the excised material is specifically recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

Although the invention has been described with reference to embodiments and examples, it should be understood that numerous and various modifications can be made without departing from the spirit of the invention. Accordingly, the invention is limited only by the following claims.

What is claimed is:

1. A compound having the structure of the formula I: 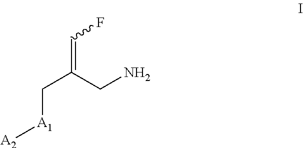
or a pharmaceutically acceptable salt thereof, wherein:
A₁ is selected from the group consisting of —(CH₂)ₙ—, —S—, —S(═O)—, —SO₂—, —O—, —C(═S)—, —C(═O)—, —NR⁵—, —C(O)NR⁵—, —S(CH₂)ₙ—, —O(CH₂)ₙ—, —NR⁵(CH₂)ₙ—, —OC(O)NR⁵—, —NHC(O)NH— —NHC(S)NH—, —NHC(S)O—, —NHC(S)—, —NR⁵SO₂—, and a bond;
when A₁ is —S(═O)—, —SO₂—, —C(═S)—, —C(═O)—, —NR⁵—, —C(O)NR⁵—, —S(CH₂)ₙ—, —O(CH₂)ₙ—, —NR⁵(CH₂)ₙ—, —OC(O)NR⁵—, —NHC(O)NH— —NHC(S)NH—, —NHC(S)O—, —NHC(S)—, or —NR₅SO₂—, A₂ is selected from the group consisting of 3-10 membered heterocyclyl optionally substituted with one or more R⁴, C₆₋₁₀ aryl optionally substituted with one or more R⁴, 5-10 membered heteroaryl optionally substituted with one or more R⁴, C₃₋₁₀ carbocyclyl optionally substituted with one or more R⁴, and C₆₋₁₀ aryl(C₁-C₆)alkyl optionally substituted with one or more R⁴;
when A₁ is a bond, A₂ is selected from the group consisting of —SO₂NR⁵R⁶, —(CH₂)ₙSO₂NR⁵R⁶, —(CH₂)ₙNR⁵SO₂R⁶, —NRSO₂R⁶, C₆₋₁₀ aryl substituted with one or more R³, 3-10 membered heterocyclyl optionally substituted with one or more R⁴, C₃₋₁₀ carbocyclyl optionally substituted with one or more R^{3a}, and —NR¹R² wherein R¹ and R² together with the nitrogen to which they are attached form a 3-10 membered heterocyclyl optionally substituted with one or more R⁴, or a 5-10 membered heteroaryl optionally substituted with one or more R⁴;
when A₁ is —O—, A₂ is selected from the group consisting of 3-10 membered heterocyclyl optionally substituted with one or more R⁴, C₆₋₁₀ aryl substituted with one or more R^{3a}, 5-10 membered heteroaryl optionally substituted with one or more R⁷, C₃₋₁₀ carbocyclyl optionally substituted with one or more R⁴, and C₆₋₁₀ aryl(C₁-C₆)alkyl optionally substituted with one or more R⁴;
when A₁ is —(CH₂)ₙ—, A₂ is selected from the group consisting of 3-10 membered heterocyclyl optionally substituted with one or more R⁴, C₆₋₁₀ aryl substituted with one or more R^{3a}, 5-10 membered heteroaryl optionally substituted with one or more R⁷, C₃₋₁₀ carbocyclyl optionally substituted with one or more R⁴, and C₆₋₁₀ aryl(C₂-C₆)alkyl optionally substituted with one or more R⁴; and
when A₁ is —S—, A₂ is selected from the group consisting of 3-10 membered heterocyclyl optionally substituted with one or more R⁴, C₆₋₁₀ aryl substituted with one or more R^{3a}, 5-10 membered heteroaryl optionally substituted with one or more R⁴, C₃₋₁₀ carbocyclyl optionally substituted with one or more R⁴, and C₆₋₁₀ aryl(C₁-C₆)alkyl optionally substituted with one or more R⁴;
each R³ is independently selected from 5-10 membered heterocyclyl (optionally substituted with —OH, halo, C₁-C₆ alkyl, C₁-C₆ alkoxy, C₁-C₆ haloalkyl, and C₁-C₆ haloalkoxy), 5-10 membered heteroaryl (optionally substituted with —OH, halo, C₁-C₆ alkyl, C₁-C₆ alkoxy, C₁-C₆ haloalkyl, and C₁-C₆ haloalkoxy), —NR⁵COOR⁶, —NR⁵COR⁶, —NR⁵CONR⁵R⁶, —NR⁵CSNR⁵R⁶, —O(CO)NR⁵R⁶, and C₃₋₇ carbocyclyl (optionally substituted with —OH, halo, C₁-C₆ alkyl, C₁-C₆ alkoxy, C₁-C₆ haloalkyl, and C₁-C₆ haloalkoxy);
each R^{3a} is independently selected from 5-10 membered heterocyclyl (optionally substituted with —OH, halo, C₁-C₆ alkyl, C₁-C₆ alkoxy, C₁-C₆ haloalkyl, and C₁-C₆ haloalkoxy), aryl (optionally substituted with —OH, halo, C₁-C₆ alkyl, C₁-C₆ alkoxy, C₁-C₆ haloalkyl, and C₁-C₆ haloalkoxy), 5-10 membered heteroaryl (optionally substituted with —OH, halo, C₁-C₆ alkyl, C₁-C₆ alkoxy, C₁-C₆ haloalkyl, and C₁-C₆ haloalkoxy), —NR⁵COOR⁶, —NR⁵COR⁶, —NR⁵CONR⁵R⁶, —NR⁵CSNR⁵R⁶, —O(CO)NR⁵R⁶, and C₃₋₇ carbocyclyl (optionally substituted with —OH, halo, C₁-C₆ alkyl, C₁-C₆ alkoxy, C₁-C₆ haloalkyl, and C₁-C₆ haloalkoxy);
each R⁴ is independently selected from the group consisting of C₁-C₆ alkyl, C₁-C₆ alkenyl, C₁-C₆ alkynyl, C₁-C₆ heteroalkyl, C₃-C₇ carbocyclyl (optionally substituted with —OH, halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with —OH, halo, C₁-C₆ alkyl, C₁-C₆ alkoxy, C₁-C₆ haloalkyl, and C₁-C₆ haloalkoxy), 5-10 membered heterocyclyl (optionally substituted with —OH, halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with —OH, halo, C₁-C₆ alkyl, C₁-C₆ alkoxy, C₁-C₆ haloalkyl, and C₁-C₆ haloalkoxy), aryl (optionally substituted with —OH, halo, C₁-C₆ alkyl, C₁-C₆ alkoxy, C₁-C₆ haloalkyl, and C₁-C₆ haloalkoxy), C₆₋₁₀ aryl(C₁-C₆)alkyl (optionally substituted with —OH, halo, C₁-C₆ alkyl, C₁-C₆ alkoxy, C₁-C₆ haloalkyl, and C₁-C₆ haloalkoxy), 5-10 membered heteroaryl (optionally substituted with —OH, halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with —OH, halo, C₁-C₆ alkyl, C₁-C₆ alkoxy, C₁-C₆ haloalkyl, and C₁-C₆ haloalkoxy), halo, cyano, C₁-C₆ alkoxy, C₁-C₆ alkoxy(C₁-C₆)alkyl), —NR⁵COOR⁶, —NR⁵COR⁶, —NR⁵CONR⁵R⁶, —NR⁵CSNR⁵R⁶, —O(CO)NR⁵R⁶, —NR⁵R⁶, —SO₂NR⁵R⁶, —C(O)NR⁵R⁶, —NSO₂R⁶, —OCF₃, —CF₃, —OH, and —SR⁵;
each R⁵ and R⁶ are independently selected from the group consisting of —H, optionally substituted C₁₋₄ alkyl, —CO-(optionally substituted C₁₋₄ alkyl), —CO-(optionally substituted C₆₋₁₀ aryl), optionally substituted C₁₋₈ alkoxyalkyl, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl; and
R⁷ is independently selected from the group consisting of C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), C₆₋₁₀ aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, C₁-C₆ alkoxy, C₁-C₆ alkoxy(C₁-C₆)alkyl), —NR⁵COOR⁶, —NR⁵CONR⁵R⁶, —O(CO)NR⁵R⁶, —NR⁵R⁶, —SO₂NR⁵R⁶, —C(O)NR⁵R⁶, —NSO₂R⁶, —OCF₃, CF₃, —OH, and —SR⁵; and
n is selected to be an integer from 1 to 2.

2. The compound of claim 1 having the structure of formula I-a: 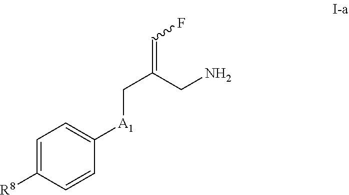
or a pharmaceutically acceptable salt thereof, wherein:
A₁ is selected from the group consisting of —CH₂—, —O—, —NH—, —S—, —NHCONH—, and —O(CO)NH—; and
R⁸ is selected from the group consisting of —NR⁵COOR⁶, —NR⁵COR⁶, —NR⁵CONR⁵R⁶, —O(CO)NR⁵R⁶, C₆₋₁₀ aryl substituted with one or more R^{3a}, and 5-10 membered heteroaryl (optionally substituted with one or more —OH, halo, C₁-C₆ alkyl, or C₁-C₆ alkoxy).

3. The compound of claim 2, wherein A₁ is selected from the group consisting of —O—, —NH—, and —S—.

4. The compound of claim 2, wherein R⁸ is selected from the group consisting of —NHCONHⁱPr, —NHCONEt₂, —N(Me)CONHEt, —NHCOOEt, —NHCOEt, and —NHCONHEt.

5. The compound of claim 2, wherein R⁸ is selected from the group consisting of

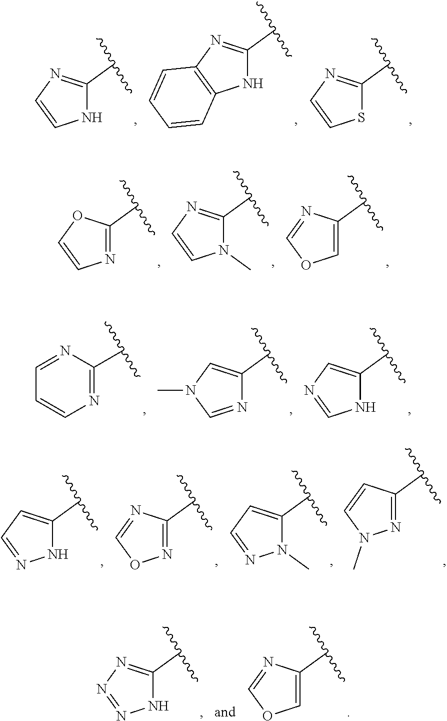

6. The compound of claim 1 having the structure of formula I-b: 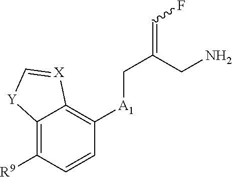
or a pharmaceutically acceptable salt thereof, wherein:
A₁ is selected from the group consisting of —CH₂—, —O—, NH, —S—, —SO₂, —NHCONH—, and —O(CO)NH—;
X is selected from the group consisting of ═N— and —CH—;
Y is selected from the group consisting of —NR—, —O—, and —S—; and
R⁹ is selected from the group consisting of —C(O)NR⁵R⁶, —NR⁵COOR⁶, —NR⁵CONR⁵R⁶, —O(CO)NR⁵R⁶, and —SO₂NR⁵R⁶.

7. The compound of claim 6, wherein X is ═N— and Y is —NH—.

8. The compound of claim 6, wherein X is ═N— and Y is —S—.

9. The compound of claim 1 having the structure of formula I-c: 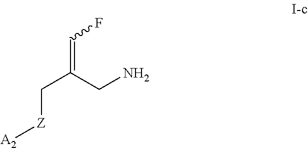
or a pharmaceutically acceptable salt thereof, wherein:
Z is selected from the group consisting of —O—, —OCH₂—, —NR⁵, —NR⁵(CH₂)ₙ—, —S—, —S(═O)—, —SO₂—, —C(═S)—, —C(═O)—, —C(O)NR⁵—, —S(CH₂)ₙ—, —OC(O)NR⁵—, —NHC(O)NH— —NHC(S)NH—, —NHC(S)O—, —NHC(S)—, and —NR⁵SO₂—; and,
A₂ is selected from the group consisting of 3-10 membered heterocyclyl optionally substituted with one or more R⁴, C₆₋₁₀ aryl substituted with one or more R^{3a}, 5-10 membered heteroaryl optionally substituted with one or more R⁷, C₃₋₁₀ carbocyclyl optionally substituted with one or more R⁴, and C₆₋₁₀ aryl(C₁-C₆)alkyl optionally substituted with one or more R⁴.

10. The compound of claim 9, wherein Z is selected from the group consisting of —O—, —NH, —NCOCH₃, and —OC(O)NH—.

11. The compound of claim 9, wherein A₂ is

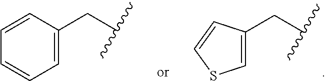 [SMILES: *Cc1ccccc1 ; *Cc1ccsc1]

12. The compound of claim 1 having the structure of formula I-d: 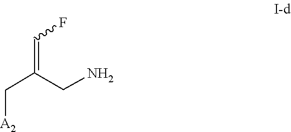
or a pharmaceutically acceptable salt thereof, wherein:
A₂ is selected from the group consisting of —SO₂NR⁵R⁶, —(CH₂)ₙSO₂NR⁵R⁶, —(CH₂)ₙNR⁵SO₂R⁶, and —NR⁵SO₂R⁶.

13. The compound of claim 12, wherein A₂ is selected from the group consisting of —NHSO₂Me, and —NHSO₂Ph.

14. The compound of claim 1 having the structure of formula I-e: 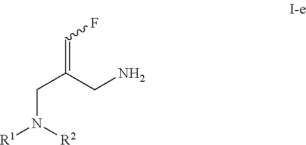
or a pharmaceutically acceptable salt thereof, wherein:
NR¹R² wherein R¹ and R² together with the nitrogen to which they are attached form a 3-10 membered heterocyclyl optionally substituted with one or more R⁴, or a 5-10 membered heteroaryl optionally substituted with one or more R⁴.

15. The compound of claim 14, wherein:
R¹ and R² together with the nitrogen to which they are attached form a ring selected from the group consisting of 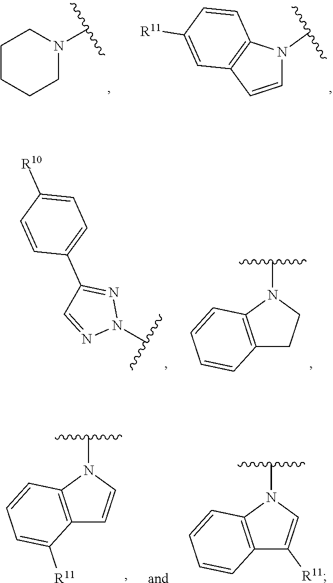
R¹¹ is selected from the group consisting of H, —OH, —C(O)NR⁵R⁶, —NR⁵COOR⁶, —NR⁵CONR⁵R⁶, and —O(CO)NR⁵R⁶, and —SO₂NR⁵R⁶, —NR⁵R⁶, —NSO₂R⁶, —NR⁵COR⁶, —OCF₃, —CF₃, and —SR⁵; and
R¹⁰ is selected from the group consisting of H, —OH, halo, C₁-C₆ alkyl, C₁-C₆ alkoxy, C₁-C₆ haloalkyl, and C₁-C₆ haloalkoxy.

16. The compound of claim 15, wherein R¹¹ is selected from the group consisting of H, —C(O)NR⁵R⁶, —NR⁵COOR⁶, —NR⁵CONR⁵R⁶, and —O(CO)NR⁵R⁶, and —SO₂NR⁵R⁶.

17. The compound of claim 1 having the structure of formula I-f: 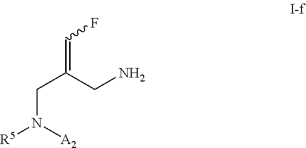
or a pharmaceutically acceptable salt thereof, wherein:
R⁵ is selected from —H, optionally substituted C₁₋₄ alkyl, —CO-(optionally substituted C₁₋₄ alkyl), —CO-(optionally substituted C₆₋₁₀ aryl), optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl; and
R⁴ is selected from halo, cyano, hydroxy, C₁-C₆ alkoxy, C₁-C₆ alkoxy(C₁-C₆)alkyl), —NR⁵COOR⁶, —NR⁵COR⁶, —NR⁵CONR⁵R⁶, —O(CO)NR⁵R⁶, —NR⁵R⁶, —SO₂NR⁵R⁶, —C(O)NR⁵R⁶, —NSO₂R⁶, —OCF₃, —CF₃, —OH, and —SR⁵.

18. The compound of claim 17, wherein R⁵ is selected from the group consisting of H, —COMe, —COPh, —CH₂Ph, phenyl, and cyclohexyl.

19. The compound of claim 17, wherein R⁴ is —CONH^{t}Bu.

20. The compound of claim 1, wherein A₁ is selected from the group consisting of —S—, —S(═O)—, —SO₂—, —O—, —C(═S)—, —C(═O)—, —NR⁵—, —C(O)NR⁵—, —S(CH₂)ₙ—, —O(CH₂)ₙ—, —NR⁵(CH₂)ₙ—, —OC(O)NR⁵—, —NHC(O)NH— —NHC(S)NH—, —NHC(S)O—, —NHC(S)—, and —NR⁵SO₂—.

21. The compound of claim 1, having the structure selected from the group consisting of: 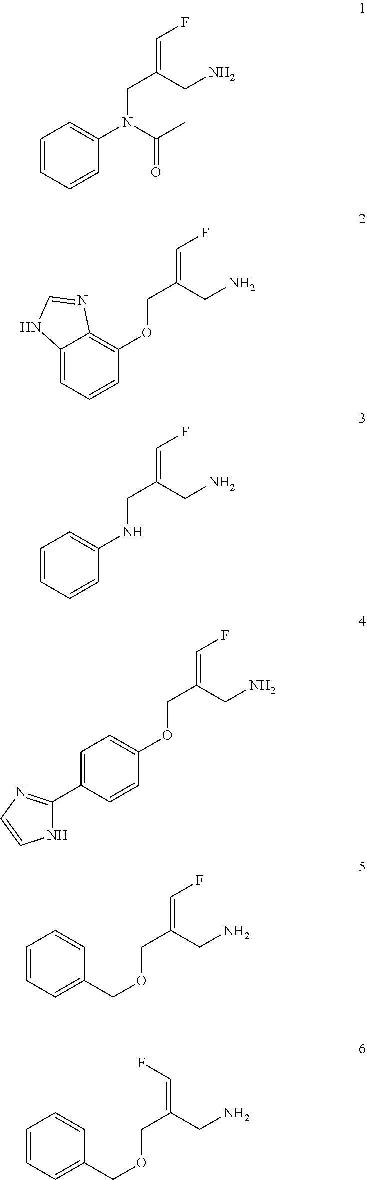 [SMILES: CC(=O)N(C/C(=C/F)CN)c1ccccc1 ; NC/C(=C\F)COc1cccc2[nH]cnc12 ; NC/C(=C\F)CNc1ccccc1 ; NC/C(=C\F)COc1ccc(-c2ncc[nH]2)cc1 ; NC/C(=C\F)COCc1ccccc1 ; NC/C(=C/F)COCc1ccccc1] 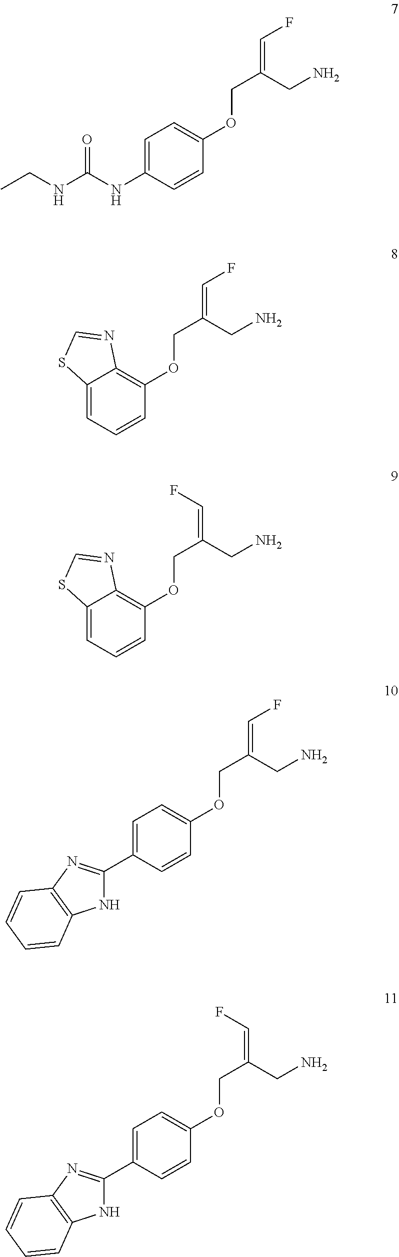 [SMILES: CCNC(=O)Nc1ccc(OC/C(=C/F)CN)cc1 ; NC/C(=C\F)COc1cccc2scnc12 ; NC/C(=C/F)COc1cccc2scnc12 ; NC/C(=C\F)COc1ccc(-c2nc3ccccc3[nH]2)cc1 ; NC/C(=C/F)COc1ccc(-c2nc3ccccc3[nH]2)cc1] 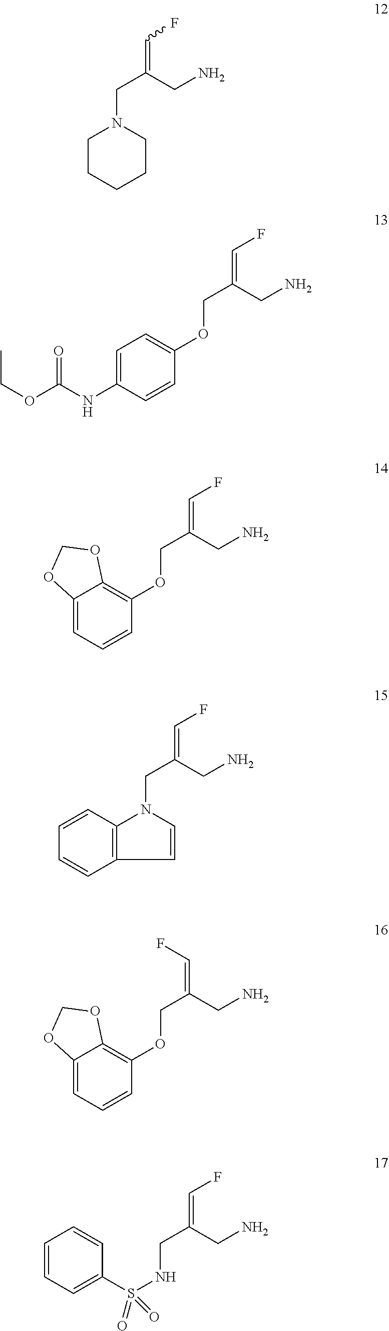 [SMILES: NCC(=CF)CN1CCCCC1 ; CCOC(=O)Nc1ccc(OC/C(=C/F)CN)cc1 ; NC/C(=C\F)COc1cccc2c1OCO2 ; NC/C(=C\F)Cn1ccc2ccccc21 ; NC/C(=C/F)COc1cccc2c1OCO2 ; NC/C(=C\F)CNS(=O)(=O)c1ccccc1] 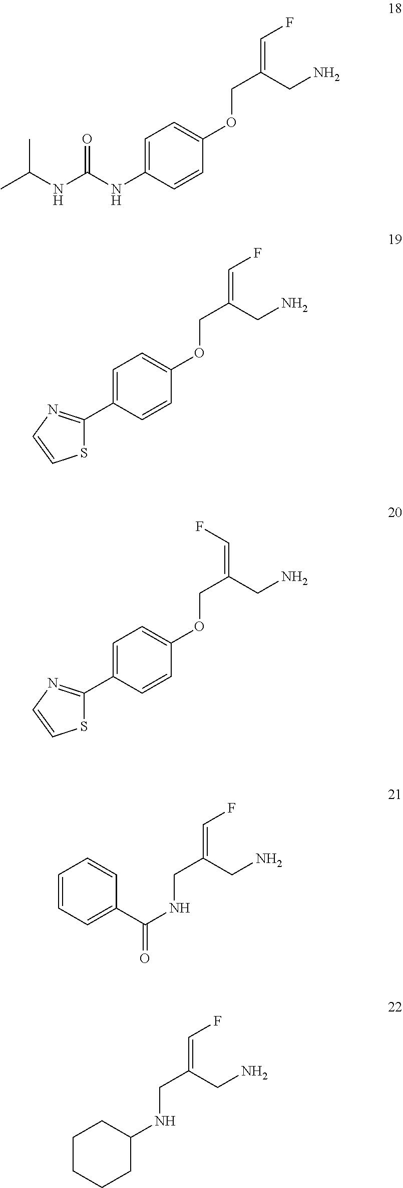 [SMILES: CC(C)NC(=O)Nc1ccc(OC/C(=C/F)CN)cc1 ; NC/C(=C\F)COc1ccc(-c2nccs2)cc1 ; NC/C(=C/F)COc1ccc(-c2nccs2)cc1 ; NC/C(=C\F)CNC(=O)c1ccccc1 ; NC/C(=C\F)CNC1CCCCC1] 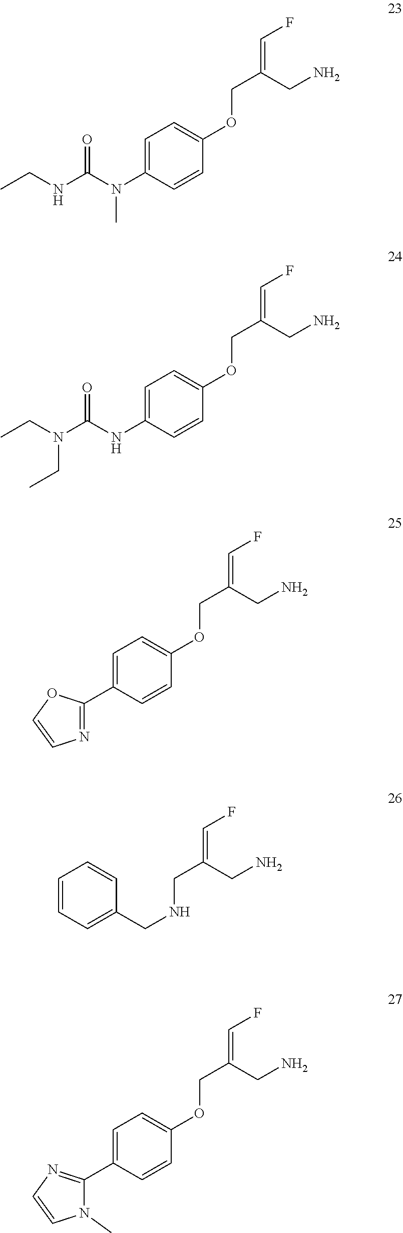 [SMILES: CCNC(=O)N(C)c1ccc(OC/C(=C/F)CN)cc1 ; CCN(CC)C(=O)Nc1ccc(OC/C(=C/F)CN)cc1 ; NC/C(=C\F)COc1ccc(-c2ncco2)cc1 ; NC/C(=C\F)CNCc1ccccc1 ; Cn1ccnc1-c1ccc(OC/C(=C/F)CN)cc1] 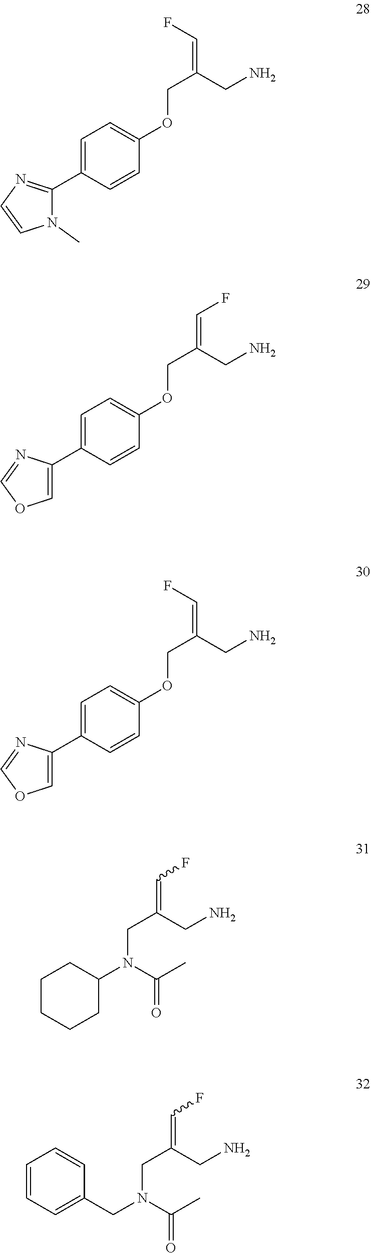 [SMILES: Cn1ccnc1-c1ccc(OC/C(=C\F)CN)cc1 ; NC/C(=C\F)COc1ccc(-c2cocn2)cc1 ; NC/C(=C/F)COc1ccc(-c2cocn2)cc1 ; CC(=O)N(CC(=CF)CN)C1CCCCC1 ; CC(=O)N(CC(=CF)CN)Cc1ccccc1] 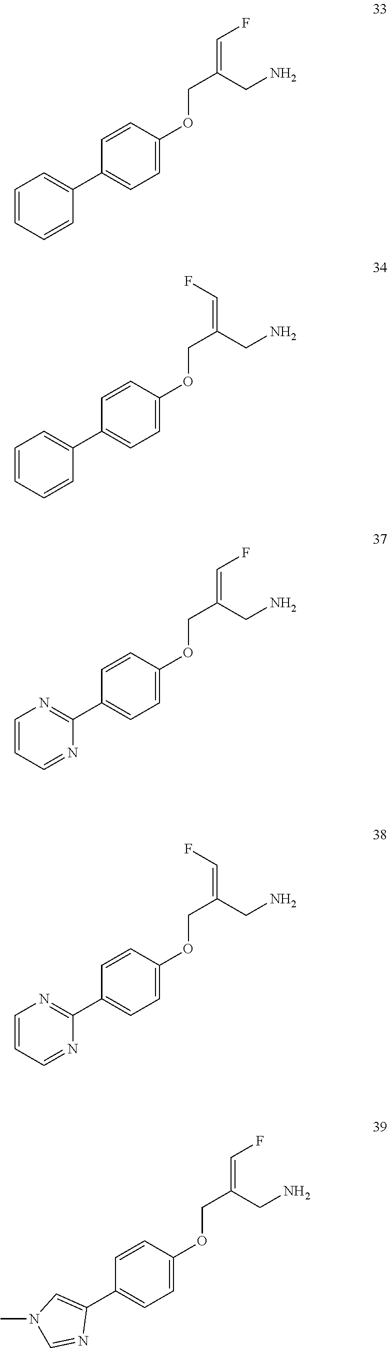 [SMILES: NC/C(=C\F)COc1ccc(-c2ccccc2)cc1 ; NC/C(=C/F)COc1ccc(-c2ccccc2)cc1 ; NC/C(=C\F)COc1ccc(-c2ncccn2)cc1 ; NC/C(=C/F)COc1ccc(-c2ncccn2)cc1 ; Cn1cnc(-c2ccc(OC/C(=C/F)CN)cc2)c1] 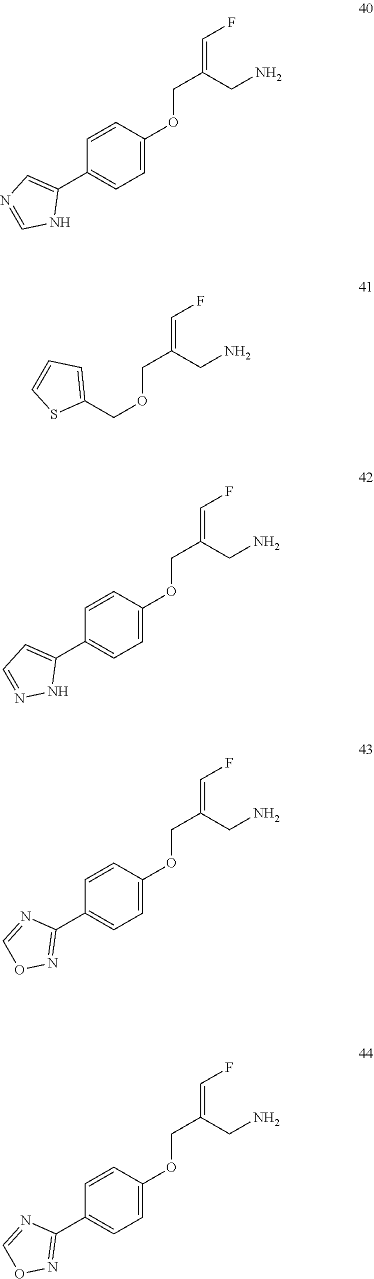 [SMILES: NC/C(=C\F)COc1ccc(-c2cnc[nH]2)cc1 ; NC/C(=C\F)COCc1cccs1 ; NC/C(=C\F)COc1ccc(-c2ccn[nH]2)cc1 ; NC/C(=C\F)COc1ccc(-c2ncon2)cc1 ; NC/C(=C\F)COc1ccc(-c2ncon2)cc1] 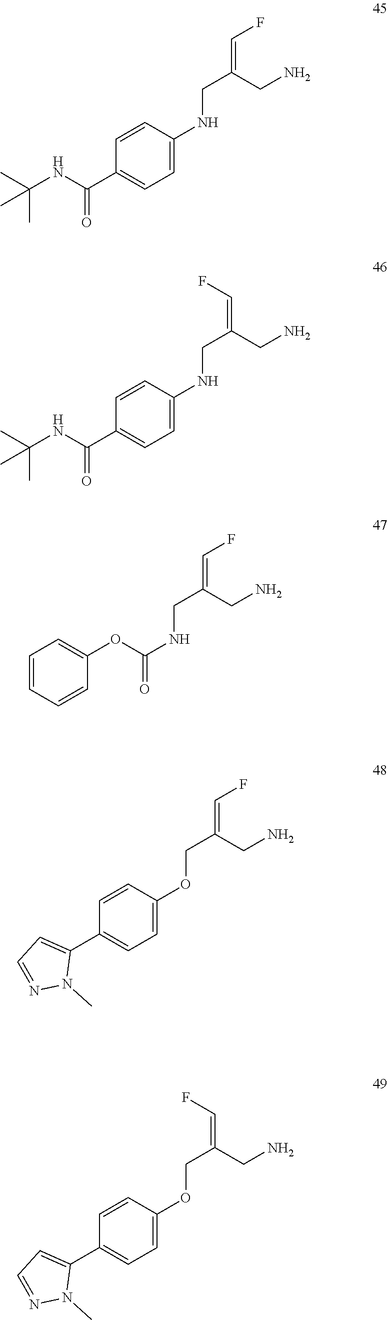 [SMILES: CC(C)(C)NC(=O)c1ccc(NC/C(=C/F)CN)cc1 ; CC(C)(C)NC(=O)c1ccc(NC/C(=C\F)CN)cc1 ; NC/C(=C\F)CNC(=O)Oc1ccccc1 ; Cn1nccc1-c1ccc(OC/C(=C/F)CN)cc1 ; Cn1nccc1-c1ccc(OC/C(=C\F)CN)cc1] 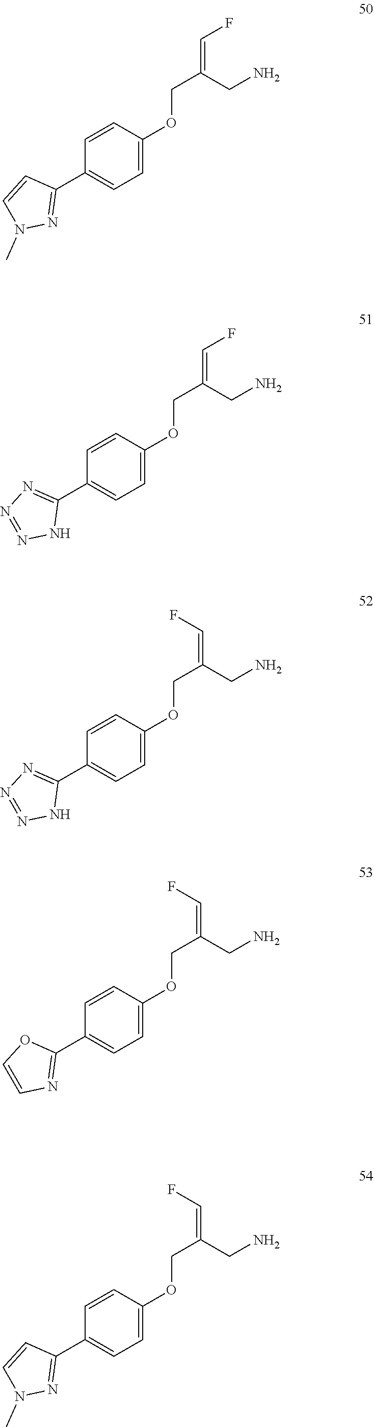 [SMILES: Cn1ccc(-c2ccc(OC/C(=C/F)CN)cc2)n1 ; NC/C(=C\F)COc1ccc(-c2nnn[nH]2)cc1 ; NC/C(=C/F)COc1ccc(-c2nnn[nH]2)cc1 ; NC/C(=C/F)COc1ccc(-c2ncco2)cc1 ; Cn1ccc(-c2ccc(OC/C(=C\F)CN)cc2)n1] 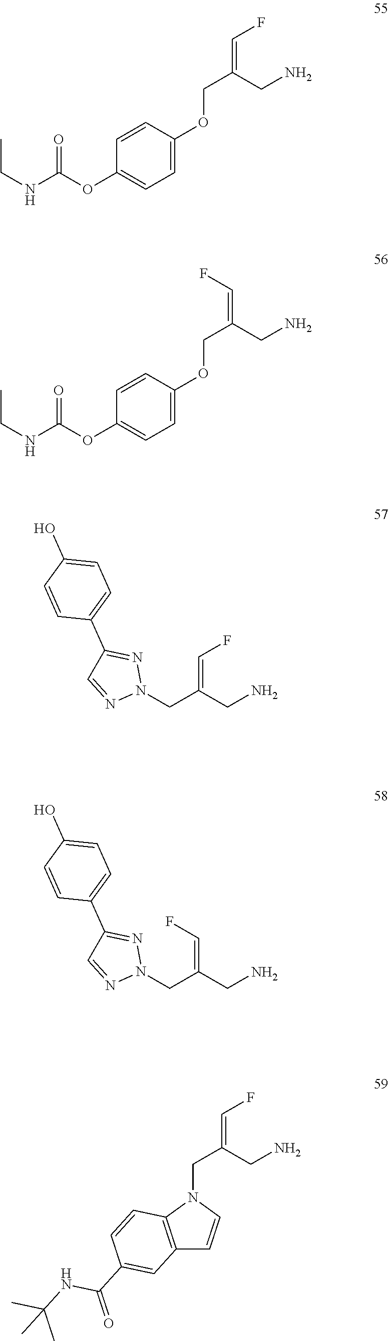 [SMILES: CCNC(=O)Oc1ccc(OC/C(=C/F)CN)cc1 ; CCNC(=O)Oc1ccc(OC/C(=C\F)CN)cc1 ; NC/C(=C\F)Cn1ncc(-c2ccc(O)cc2)n1 ; NC/C(=C/F)Cn1ncc(-c2ccc(O)cc2)n1 ; CC(C)(C)NC(=O)c1ccc2c(ccn2C/C(=C/F)CN)c1] 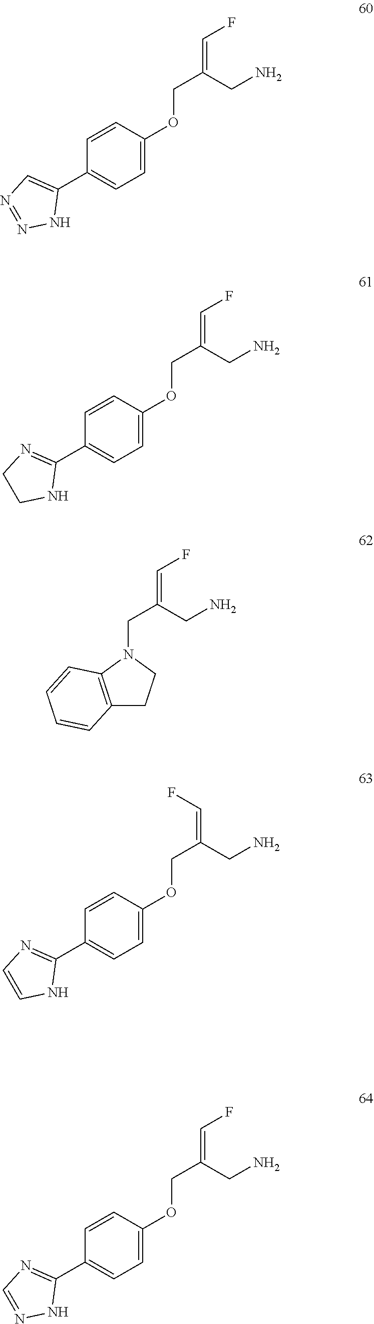 [SMILES: NC/C(=C\F)COc1ccc(-c2cnn[nH]2)cc1 ; NC/C(=C\F)COc1ccc(C2=NCCN2)cc1 ; NC/C(=C\F)CN1CCc2ccccc21 ; NC/C(=C/F)COc1ccc(-c2ncc[nH]2)cc1 ; NC/C(=C\F)COc1ccc(-c2ncn[nH]2)cc1] 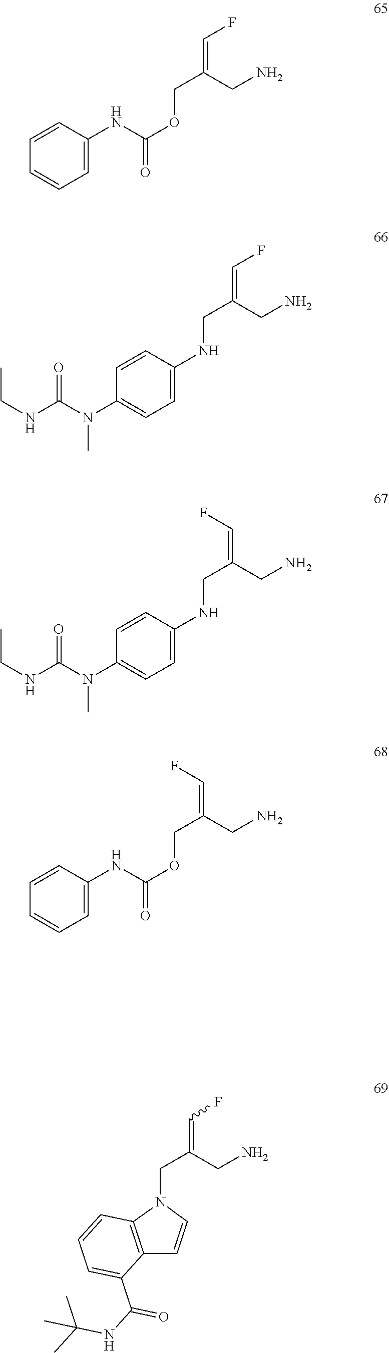 [SMILES: NC/C(=C\F)COC(=O)Nc1ccccc1 ; CCNC(=O)N(C)c1ccc(NC/C(=C/F)CN)cc1 ; CCNC(=O)N(C)c1ccc(NC/C(=C\F)CN)cc1 ; NC/C(=C/F)COC(=O)Nc1ccccc1 ; CC(C)(C)NC(=O)c1cccc2c1ccn2CC(=CF)CN] 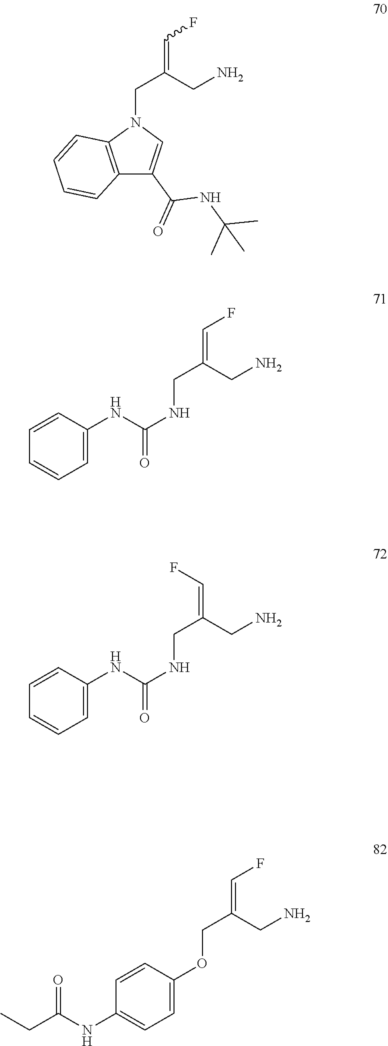
and pharmaceutically acceptable salts thereof.

22. A pharmaceutical composition comprising a therapeutically effective amount of a compound of any one of claims 1-21 and a pharmaceutically acceptable excipient.

23. A method of treating fibrotic disease or a secondary disease state or condition thereof, comprising administering to a subject in need thereof, a compound according to any one of claims 1-21.

24. The method of claim 23, wherein the disease is selected from the group consisting of progressive liver fibrosis, renal fibrosis, idiopathic lung fibrosis, diabetic nephropathy, systemic sclerosis, idiopathic pulmonary fibrosis, non-alcoholic steatohepatitis, primary sclerosing cholangitis, corneal fibrosis, liver cirrhosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis.

25. A method of treating a liver disorder, comprising administering to a subject in need thereof, a compound according to any one of claims 1-21.

26. The method of claim 25, wherein the liver disorder is non-alcoholic steatohepatitis.

27. The method of claim 23, wherein the treatment inhibits myofibroblast differentiation or treats a disease associated with myofibroblast differentiation.

28. The method of claim 23, wherein the treatment inhibits Fibroblast-to-Myofibroblast Transition (FMT).

29. The method of claim 23, wherein the treatment inhibits Epithelial to Mesenchymal Transition or Endothelial to Mesenchymal Transition.

30. The method of claim 29 wherein the myofibroblast differentiation is a TGFβ-mediated myofibroblast differentiation.

31. The method of claim 23, wherein the treatment decreases the expression level and/or activity of vascular adhesion protein-1 (VAP-1).

32. The method of claim 23, wherein the compound is of Formula I.

33. The method of claim 23, wherein the subject is a mammal.

34. The method of claim 23, wherein the subject is a human.

35. The method of claim 23, wherein the route of administration is selected from the group consisting of: enteral, intravenous, oral, intraarticular, intramuscular, subcutaneous, intraperitoneal, epidural, transdermal, and transmucosal.

36. The method of claim 23, wherein the administration is intravenous.

37. A method of inhibiting myofibroblast differentiation comprising contacting a cell with a compound of anyone of claims 1-21.

38. The method of claim 37, wherein the cell is in a fibrotic tissue.

39. The method of claim 37 wherein the cell is in a tissue with high TGFβ signaling.

40. A method for inhibiting VAP-1, the method comprising contacting a compound of any one of claims 1-21 with a VAP-1 enzyme residing inside a subject.
